SEQUENCE LISTING (120 sequences)

SEQ ID NO: 1 - DNA, 21 bp, human
gggagcagagaattctcttat

SEQ ID NO: 2 - DNA, 21 bp, human
ggagcagagaattctcttatc

SEQ ID NO: 3 - DNA, 21 bp, human
gagcagagaattctcttatcc

SEQ ID NO: 4 - DNA, 21 bp, human
gctacctggagcttcttaaca

SEQ ID NO: 5 - DNA, 21 bp, human
ggagcttcttaacagcgatgc

SEQ ID NO: 6 - DNA, 21 bp, human
gggtctccagtatattcatct

SEQ ID NO: 7 - DNA, 21 bp, human
gcctcctgatgcacatgtact

SEQ ID NO: 8 - DNA, 21 bp, human
ggaagacctgggaaagcttgt

SEQ ID NO: 9 - DNA, 21 bp, human
ggctaagcttgtacaataact

SEQ ID NO: 10 - DNA, 21 bp, human
gcggaatgaaccacatcttgc

SEQ ID NO: 11 - DNA, 21 bp, human
ggccttctctgaaggacattg

SEQ ID NO: 12 - DNA, 21 bp, human
ggactcaatgcactgacattg

SEQ ID NO: 13 - DNA, 21 bp, human
ggtacccactgctctggttat

SEQ ID NO: 14 - DNA, 21 bp, human
gctcccactccatgaggtatt

SEQ ID NO: 15 - DNA, 21 bp, human
ggtatttcttcacatccgtgt

SEQ ID NO: 16 - DNA, 21 bp, human
aggagacacggaatgtgaagg

SEQ ID NO: 17 - DNA, 19 bp, human
ggttgatgagttaattaaa

SEQ ID NO: 18 - DNA, 21 bp, human
gctcccactccatgaggtatt

SEQ ID NO: 19 - DNA, 21 bp, human
ggtatttctacacctccgtgt

SEQ ID NO: 20 - DNA, 21 bp, human
ggaccggaacacacagatcta

SEQ ID NO: 21 - DNA, 21 bp, human
accggaacacacagatctaca

SEQ ID NO: 22 - DNA, 21 bp, human
ggaacacacagatctacaagg

SEQ ID NO: 23 - DNA, 21 bp, human
gaacacacagatctacaaggc

SEQ ID NO: 24 - DNA, 9 bp, Artificial sequence
  synthesized
ttcaagaga

SEQ ID NO: 25 - DNA, 119 bp, Artificial sequence
  synthesized
gaggcttcagtactttacagaatcgttgcctgcacatcttggaaacacttgctgggattacttcttcaggttaacccaacagaaggctaaagaaggtatattgctgttgacagtgagcg

SEQ ID NO: 26 - DNA, 19 bp, Artificial sequence
  synthesized
tagtgaagccacagatgta

SEQ ID NO: 27 - DNA, 119 bp, Artificial sequence
  synthesized
tgcctactgcctcggacttcaaggggctactttaggagcaattatcttgtttactaaaactgaataccttgctatctctttgatacatttttacaaagctgaattaaaatggtataaat

SEQ ID NO: 28 - DNA, 119 bp, Artificial sequence
  synthesized
gcatacacaaacatttctttctctcttgcaggtggcacaaaccaggaaggggaaatctgtggtttaaattctttatgcctcatcctctgagtgctgaaggcttgctgtaggctgtatgc

SEQ ID NO: 29 - DNA, 19 bp, Artificial sequence
  synthesized
tagtgaagccacagatgta

SEQ ID NO: 30 - DNA, 119 bp, Artificial sequence
  synthesized
gtgtatgatgcctgttactagcattcacatggaacaaattgctgccgtgggaggatgacaaagaagcatgagtcaccctgctggataaacttagacttcaggctttatcatttttcaat

SEQ ID NO: 31 - DNA, 21 bp, human
ggatggatttgattatgatcc

SEQ ID NO: 32 - DNA, 21 bp, human
ggaccttggaacaatggattg

SEQ ID NO: 33 - DNA, 21 bp, human
gctaattcttgctgaacttct

SEQ ID NO: 34 - DNA, 21 bp, human
gctgaacttcttcatgtatgt

SEQ ID NO: 35 - DNA, 21 bp, human
gcctcatctctttgttctaaa

SEQ ID NO: 36 - DNA, 21 bp, human
gctctggagaagatatatttg

SEQ ID NO: 37 - DNA, 21 bp, human
gctcttgagggaactaataga

SEQ ID NO: 38 - DNA, 21 bp, human
gggacggcattaatgtattca

SEQ ID NO: 39 - DNA, 21 bp, human
ggacaaacatgcaaactatag

SEQ ID NO: 40 - DNA, 21 bp, human
gcagcaaccagctaccattct

SEQ ID NO: 41 - DNA, 21 bp, human
gcagttctgttgccactctct

SEQ ID NO: 42 - DNA, 21 bp, human
gggagagttcatccaggaaat

SEQ ID NO: 43 - DNA, 21 bp, human
ggagagttcatccaggaaatt

SEQ ID NO: 44 - DNA, 21 bp, human
gagagttcatccaggaaatta

SEQ ID NO: 45 - DNA, 21 bp, human
gcctgtcaaagagagagagca

SEQ ID NO: 46 - DNA, 21 bp, human
gctcagcttcgtactgagttc

SEQ ID NO: 47 - DNA, 21 bp, human
gcttcacagaactacagagag

SEQ ID NO: 48 - DNA, 21 bp, human
gcatctactggacaaagtatt

SEQ ID NO: 49 - DNA, 21 bp, human
ggctgaattacccatgcttta

SEQ ID NO: 50 - DNA, 21 bp, human
gctgaattacccatgctttaa

SEQ ID NO: 51 - DNA, 21 bp, human
gggttggtttatccaggaata

SEQ ID NO: 52 - DNA, 21 bp, human
ggatcagaagagaagccaacg

SEQ ID NO: 53 - DNA, 21 bp, human
ggttcaccatccaggtgttca

SEQ ID NO: 54 - DNA, 21 bp, human
gctctcttctctggaactaac

SEQ ID NO: 55 - DNA, 21 bp, human
gctagagtgactccatcttaa

SEQ ID NO: 56 - DNA, 21 bp, human
gctgaccaccaattataattg

SEQ ID NO: 57 - DNA, 21 bp, human
gcagaatatttaaggccatac

SEQ ID NO: 58 - DNA, 21 bp, human
gcccacttaaaggcagcatta

SEQ ID NO: 59 - DNA, 21 bp, human
ggtcatcaataccactgttaa

SEQ ID NO: 60 - DNA, 21 bp, human
gcattcctccttctcctttct

SEQ ID NO: 61 - DNA, 21 bp, human
ggaggaactttgtgaacattc

SEQ ID NO: 62 - DNA, 21 bp, human
gctgtaagaaggatgctttca

SEQ ID NO: 63 - DNA, 21 bp, human
gctgcaggcaggattgtttca

SEQ ID NO: 64 - DNA, 21 bp, human
gcagttcgaggtcaagtttga

SEQ ID NO: 65 - DNA, 21 bp, human
gccaattagctgagaagaatt

SEQ ID NO: 66 - DNA, 21 bp, human
gcaggtttacagtgtatatgt

SEQ ID NO: 67 - DNA, 21 bp, human
gcctacagagactagagtagg

SEQ ID NO: 68 - DNA, 21 bp, human
gcagttgggtaccttccattc

SEQ ID NO: 69 - DNA, 21 bp, human
gcaactcaggtgcatgataca

SEQ ID NO: 70 - DNA, 21 bp, human
gcatggcgctggtacgtaaat

SEQ ID NO: 71 - DNA, 19 bp, human
gcctcgagtttgagagcta

SEQ ID NO: 72 - DNA, 19 bp, human
agacattctggatgagtta

SEQ ID NO: 73 - DNA, 19 bp, human
gggtctgttacccaaagaa

SEQ ID NO: 74 - DNA, 19 bp, human
ggtctgttacccaaagaat

SEQ ID NO: 75 - DNA, 19 bp, human
ggaaggaagcggacgctca

SEQ ID NO: 76 - DNA, 19 bp, human
ggaggcagtacttctgata

SEQ ID NO: 77 - DNA, 19 bp, human
cgctctagagctcagctga

SEQ ID NO: 78 - DNA, 19 bp, human
ccaccacctcaaccaataa

SEQ ID NO: 79 - DNA, 19 bp, human
atttcaagaagtcgatcaa

SEQ ID NO: 80 - DNA, 19 bp, human
gaagatctgattaccttca

SEQ ID NO: 81 - DNA, 21 bp, human
ggacactggttcaacacctgt

SEQ ID NO: 82 - DNA, 21 bp, human
ggttcaacacctgtgacttca

SEQ ID NO: 83 - DNA, 21 bp, human
acctgtgacttcatgtgtgcg

SEQ ID NO: 84 - DNA, 21 bp, human
gctggacgtgaccatcatgta

SEQ ID NO: 85 - DNA, 21 bp, human
ggacgtgaccatcatgtacaa

SEQ ID NO: 86 - DNA, 21 bp, human
gacgtgaccatcatgtacaag

SEQ ID NO: 87 - DNA, 21 bp, human
acgtgaccatcatgtacaagg

SEQ ID NO: 88 - DNA, 21 bp, human
acgctataccatctacctggg

SEQ ID NO: 89 - DNA, 21 bp, human
gcctctatgacgacatcgagt

SEQ ID NO: 90 - DNA, 21 bp, human
gacatcgagtgcttccttatg

SEQ ID NO: 91 - DNA, 804 bp, Artificial sequence
  synthesized
tgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggc

SEQ ID NO: 92 - DNA, 837 bp, Artificial sequence
  synthesized
actagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctct

SEQ ID NO: 93 - DNA, 1000 bp, Artificial sequence
  synthesized
catttaaaaatatcagaaacatttttaagacaatttttcaaaagctacacaataatgcttatattcataatttaggtatttgtcttggcataaagattaacatctctctatataatttcttctcattatttcctacaaaattgaagaaataaagtatttggtatttcatttatttagatcttctttcacccagaaattattgaaagtgagggtaaataaatagtttaatttaaaattcacatactgcagtataacataaggttcttgtttgaattttactgtttgccatgcattaaaaagcactcattttaaggctatggaagatgctcacgtagtatctctaagcacctatttttcagaaggatttgtggcttctgtgccacagatgataaggaattttattgttctggcttccatactcagcttgacctattccatcctaaataagctaatcctggagagagttttgcttcaagtgtaaaatgatcaacacaagaatggaaagtaaatctaccaccacacatgtaaatactgatatataggccatagtagagccttatattcatataaataaagtttcagatcatacttctccctaaaatcaagacatccaattttaagaacttctgtcctcatcattggtaacgagtaccagaaactgatgttacagcatataaaatagttctttttaaatatcatgcaactcttaccaatgttttgattcagtgataaaattatggagcttcataataaccaggaaaatgcagtgttaaaaacaacaataaaaggctagcaagaaattataaatatgttgttaagggcattggcttctttagggcttgctctcgtaactgataccaaatgggaattcagagtctaaaaatatcttctttggtaaaaggaaagtcttggccaggaagtaaataagcttttcccttaattcagcacatctccctaaatacactattatcacctaccatctggaatgaagcttccacca

SEQ ID NO: 94 - DNA, 1000 bp, Artificial sequence
  synthesized
actaatgacctgcctcattaaatatcaggaagcaaaatccacattcctaagaccaccctgagctggaatcaatcctgttctaaaaaccatcaacttttaataagagatacatctaacatagggcttacagtcataccagcctagttataagtacaaaatagccaatatcaaagttgttttgtatgaatatagatacattggtatagagaaatagaagaacacttggcacacaaacattgggttgcttcattagcatttctttttactttaagcaagatttcttgagggacatcttttctacgttggagtaaaaaatacgttatttaaacttgaatttttttttcagtaagtgaaacctatattattcagccaaatccttaagcttgagttaaataattgaaacagaaatttaaaagttataactaatggatattcagacattatacatttcttacccaagtgttataatagggacctactattttaagtgacacttaaacatttatagttgacttatagaccataaattttaatgatcataaattttgatttacacaattattgagttaacaagtcacaaagttagtctatttttataaaggtaaaaattaggaatcagatcaaaacattgtgaggtgccttctcagatgttatacccatactttctattcttaagtttagaaaataaataaaattcttactaaatctcttctcactttatcaaaaatattttgcaggttgtcctgataggaaattgagggaataatagctgttcctttggacgaggatcattggtgctattcattccaaatgtgactttatgtaattaattatcaacttcaaaccactgccattggaatcctcttttgttgcactttgtccagtatcaagtcagtgggagaatggtggagatctgtggggactgaagcaaacatacatggcttttgaggaagagcactttagattttaaactgattaacattaaacatctactgctttt

SEQ ID NO: 95 - DNA, 23 bp, Artificial sequence
  synthesized
tataaggtggtcccagctcgggg

SEQ ID NO: 96 - DNA, 23 bp, Artificial sequence
  synthesized
agggccggttaatgtggctctgg

SEQ ID NO: 97 - DNA, 23 bp, Artificial sequence
  synthesized
ggtggaagcttcattccagatgg

SEQ ID NO: 98 - DNA, 23 bp, Artificial sequence
  synthesized
gacctgcctcattaaatatcagg

SEQ ID NO: 99 - DNA, 22 bp, Artificial sequence
  synthesized
ccatagctcagtctggtctatc

SEQ ID NO: 100 - DNA, 22 bp, Artificial sequence
  synthesized
tcaggatgatctggacgaagag

SEQ ID NO: 101 - DNA, 20 bp, Artificial sequence
  synthesized
ccggtcctggactttgtctc

SEQ ID NO: 102 - DNA, 20 bp, Artificial sequence
  synthesized
ctcgacatcggcaaggtgtg

SEQ ID NO: 103 - DNA, 20 bp, Artificial sequence
  synthesized
cgcattggagtcgctttaac

SEQ ID NO: 104 - DNA, 24 bp, Artificial sequence
  synthesized
cgagctgcaagaactcttcctcac

SEQ ID NO: 105 - DNA, 23 bp, Artificial sequence
  synthesized
cacggcacttacctgtgttctgg

SEQ ID NO: 106 - DNA, 23 bp, Artificial sequence
  synthesized
cagtacaggcatccctgtgaaag

SEQ ID NO: 107 - DNA, 1101 bp, human
atgcgggtcatggcgccccgaaccctcatcctgctgctctcgggagccctggccctgaccgagacctgggcctgctcccactccatgaagtatttcttcacatccgtgtcccggcctggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccgggtctcacaccctccagtggatgtgtggctgcgacctggggcccgacgggcgcctcctccgcgggtatgaccagtacgcctacgacggcaaggattacatcgccctgaacgaggacctgcgctcctggaccgccgcggacaccgcggctcagatcacccagcgcaagtgggaggcggcccgtgaggcggagcagcggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacaccgagcttgtggagaccaggccagcaggagatggaaccttccagaagtgggcagctgtgatggtgccttctggagaagagcagagatacacgtgccatgtgcagcacgaggggctgccggagcccctcaccctgagatgggagccgtcttcccagcccaccatccccatcgtgggcatcgttgctggcctggctgtcctggctgtcctagctgtcctaggagctgtggtggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 108 - DNA, 1101 bp, human
atgcgggtcatggcgccccgaaccctcctcctgctgctctcgggagccctggccctgaccgagacctgggcctgctcccactccatgaggtatttctacaccgctgtgtcccggcccagccgcggagagccccacttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacagactgaccgagtgaacctgcggaaactacgcggctactacaaccagagcgaggccgggtctcacaccctccagaggatgtacggctgcgacctggggcccgacgggcgcctcctccgcgggtatgaccagtccgcctacgacggcaaggattacatcgccctgaacgaggacctgcgctcctggaccgccgcggacacagcggctcagatcacccagcgcaagtgggaggcggcccgtgaggcggagcagtggagagcctacctggagggcgagtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctacggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccatcttcccagcccaccatccccatcgtgggcatcgttgctggcctggctgtcctggctgtcctagctgtcctaggagctgtggtggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 109 - DNA, 1101 bp, human
atgcgggtcatggcgccccgaaccctcatcctgctgctctcgggagccctggccctgaccgagacctgggccggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccaggtctcacatcatccagaggatgtatggctgcgacgtggggcccgacgggcgcctcctccgcgggtatgaccagtacgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacactgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccgtcttcccagcccaccatccccatcgtgggcatcgttgctggcctggctgtcctggctgtcctagctgtcctaggagctgtggtggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 110 - DNA, 1101 bp, human
atgcgggtcatggcgccccgaaccctcatcctgctgctctcgggagccctggccctgaccgagacctgggccggctcccactccatgaggtatttctacaccgctgtgtcccggcccggccgcggggagccccacttcatcgcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccgggtctcacatcatccagaggatgtatggctgcgacgtggggcccgacgggcgcctcctccgcgggtatgaccagtacgcctacgacggcaaggattacatcgccctgaacgaggatctgcgctcctggaccgccgcggacacggcggctcagatcacccagcgcaagtgggaggcggcccgtgaggcggagcagctgagagcctacctggagggcctgtgcgtggagtggctccgcagatacctgaagaatgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacactgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccgtcttcccagcccaccatccccatcgtgggcatcgttgctggcctggctgtcctggctgtcctagctgtcctaggagctgtggtggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 111 - DNA, 1101 bp, human
atgcgggtcatggcgccccgaaccctcatcctgctgctctcgggagccctggccctgaccgagacctgggccggctcccactccatgaggtatttctccacatccgtgtcctggcccggccgcggggagccccgcttcatcgcagtgggctacgtggacgacacgcagttcgtgcggttcgacagcgacgccgcgagtccaagaggggagccgcgggagccgtgggtggagcaggaggggccggagtattgggaccgggagacacagaagtacaagcgccaggcacaggctgaccgagtgaacctgcggaaactgcgcggctactacaaccagagcgaggacgggtctcacaccctccagaggatgtttggctgcgacctggggccggacgggcgcctcctccgcgggtataaccagttcgcctacgacggcaaggattacatcgccctgaacgaggatctgcgctcctggaccgccgcggacacggcggctcagatcacccagcgcaagtgggaggcggcccgtgaggcggagcagcggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagtgggatggggaggaccaaactcaggacaccgagcttgtggagaccaggccagcaggagatggaaccttccagaagtgggcagctgtggtggtgccttctggagaagagcagagatacacgtgccatgttcagcacgaggggctgccggagcccctcaccctgagatggaagccgtcttcccagcccaccatccccatcgtgggcatcgttgctggcctggctgtcctggctgtcctagctgtcctaggagctatggtggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 112 - DNA, 1101 bp, human
atgcgggtcatggcgccccgaaccctcatcctgctgctctcgggagccctggccctgaccgagacctgggcctgctcccactccatgaggtatttctacaccgccgtgtcccggcccggccgcggagagccccgcttcatcgcagtgggctacgtggacgacacgcagttcgtgcagttcgacagcgacgccgcgagtccaagaggggagccgcgggcgccgtgggtggagcaggaggggccggagtattgggaccgggagacacagaagtacaagcgccaggcacagactgaccgagtgaacctgcggaaactgcgcggctactacaaccagagcgaggccgggtctcacaccctccagaggatgtatggctgcgacctggggcccgacgggcgcctcctccgcgggtataaccagttcgcctacgacggcaaggattacatcgccctgaatgaggacctgcgctcctggaccgccgcggacaaggcggctcagatcacccagcgcaagtgggaggcggcccgtgaggcggagcagcggagagcctacctggagggcacgtgcgtggagtggctccgcagatacctggagaacgggaagaagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggggccatcttcccagcccaccatccccatcgtgggcatcgttgctggcctggctgtcctggctgtcctagctgtcctaggagctgtgatggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 113 - DNA, 1101 bp, human
atgcgggtcatggcgccccgaaccctcatcctgctgctctcgggagccctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccccgggcgccgtgggtggagcaggaggggccggagtattgggaccgggagacacagaagtacaagcgccaggcacaggctgaccgagtgaacctgcggaaactgcgcggctactacaaccagagcgaggacgggtctcacaccctccagtggatgtatggctgcgacctggggcccgacgggcgcctcctccgcgggtatgaccagtccgcctacgacggcaaggattacatcgccctgaacgaggacctgcgctcctggaccgccgcggacacggcggctcagatcacccagcgcaagtgggaggcggcccgtgaggcggagcagtggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggagccatcttcccagcccaccatccccatcgtgggcatcgttgctggcctggctgtcctggctgtcctagctgtcctaggagctgtgatggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 114 - DNA, 1101 bp, human
atgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaactacaagcgccaggcacaggctgaccgagtgagcctgcggaacctgcgcggctactacaaccagagcgaggacgggtctcacaccctccagaggatgtatggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctga

SEQ ID NO: 115 - DNA, 1101 bp, human
atgcgggtcatggcgccccgagccctcctcctgctgctctcgggaggcctggccctgaccgagacctgggcctgctcccactccatgaggtatttcgacaccgccgtgtcccggcccggccgcggagagccccgcttcatctcagtgggctacgtggacgacacgcagttcgtgcggttcgacagcgacgccgcgagtccgagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggacgggtctcacaccctccagaggatgtctggctgcgacctggggcccgacgggcgcctcctccgcgggtatgaccagtccgcctacgacggcaaggattacatcgccctgaacgaggacctgcgctcctggaccgccgcggacaccgcggctcagatcacccagcgcaagttggaggcggcccgtgcggcggagcagctgagagcctacctggagggcacgtgcgtggagtggctccgcagatacctggagaacgggaaggagacgctgcagcgcgcagaacccccaaagacacacgtgacccaccaccccctctctgaccatgaggccaccctgaggtgctgggccctgggcttctaccctgcggagatcacactgacctggcagcgggatggggaggaccagacccaggacaccgagcttgtggagaccaggccagcaggagatggaaccttccagaagtgggcagctgtggtggtgccttctggacaagagcagagatacacgtgccatatgcagcacgaggggctgcaagagcccctcaccctgagctgggagccatcttcccagcccaccatccccatcatgggcatcgttgctggcctggctgtcctggttgtcctagctgtccttggagctgtggtcaccgctatgatgtgtaggaggaagagctcaggtggaaaaggagggagctgctctcaggctgcgtgcagcaacagtgcccagggctctgatgagtctctcatcacttgtaaagcctga

SEQ ID NO: 116 - DNA, 1101 bp, human
atgcgggtcatggcgccccgaaccctcatcctgctgctctcgggagccctggccctgaccgagacctgggcctgctcccactccatgaggtatttctacaccgccgtgtcccggcccggccgcggagagccccgcttcatcgcagtgggctacgtggacgacacgcagttcgtgcagttcgacagcgacgccgcgagtccaagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccgggtctcacaccctccagaggatgtatggctgcgacctggggcccgacgggcgcctcctccgcgggtataaccagttcgcctacgacggcaaggattacatcgccctgaatgaggacctgcgctcctggaccgccgcggacacggcggctcagatcacccagcgcaagtgggaggcggcccgtacggcggagcagctgagagcctacctggagggcacgtgcgtggagtggctccgcagatacctggagaacgggaagaagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccagagcccctcaccctgagatgggggccatcttcccagcccaccatccccatcgtgggcatcgttgctggcctggctgtcctggctgtcctagctgtcctaggagctgtgatggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 117 - DNA, 1101 bp, human
atgcgggtcatggcgccccgaaccctcatcctgctgctctcgggagccctggccctgaccgagacctgggcctgctcccactccatgaggtatttctacaccgccgtgtcccggcccggccgcggagagccccgcttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacaggctgaccgagtgagcctgcggaacctgcgcggctactacaaccagagcgaggccgggtctcacaccctccagaggatgtacggctgcgacctggggcccgacgggcgcctcctccgcgggtatgaccagtccgcctacgacggcaaggattacatcgccctgaacgaggacctgcgctcctggaccgctgcggacacggcggctcagatcacccagcgcaagtgggaggcggcccgtgaggcggagcagtggagagcctacctggagggcacgtgcgtggagtggctccgcagatacctggagaacgggaaggagacgctgcagcgcgcggaacacccaaagacacacgtgacccaccatcccgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccatcttcccagcccaccatccccatcgtgggcatcgttgctggcctggctgtcctggctgtcctagctgtcctaggagctgtgatggctgttgtgatgtgtaggaggaagagctcaggtggaaaaggagggagctgctctcaggctgcgtccagcaacagtgcccagggctctgatgagtctctcatcgcttgtaaagcctga

SEQ ID NO: 118 - DNA, 1101 bp, human
atgcgggtcatggcgccccgaaccctcatcctgctgctctcgggagccctggccctgaccgagacctgggcctgctcccactccatgaggtatttctacaccgccgtgtcccggcccggccgcggagagccccgcttcatcgcagtgggctacgtggacgacacgcagttcgtgcggttcgacagcgacgccgcgagtccaagaggggagccgcgggcgccgtgggtggagcaggaggggccggagtattgggaccgggagacacagaagtacaagcgccaggcacagactgaccgagtgagcctgcggaacctgcgcggctactacaaccagagcgaggccgggtctcacaccctccagtggatgtatggctgcgacctggggcccgacgggcgcctcctccgcgggtatgaccagtccgcctacgacggcaaggattacatcgccctgaacgaggacctgcgctcctggaccgccgcggacacggcggctcagatcacccagcgcaagtgggaggcggcccgtgcggcggagcagcagagagcctacctggagggcacgtgcgtggagtggctccgcagatacctggagaacgggaaggagacgctgcagcgcgcggaacacccaaagacacacgtgacccaccatctcgtctctgaccatgaggccaccctgaggtgctgggccctgggcttctaccctgcggagatcacactgacctggcagcgggatggcgaggaccaaactcaggacaccgagcttgtggagaccaggccagcaggagatggaaccttccagaagtgggcagctgtggtggtgccttctggagaagagcagagatacacgtgccatgtgcagcacgaggggctgccggagcccctcaccctgagatgggagccatcttcccagcccaccatccccatcgtgggcatcgttgctggcctggctgtcctggctgtcctagctgtcctaggagctgtggtggctgttgttatgtgtaggaggaagagctcaggtggaaaaggagggagctgctctcaggctgcgtccagcaacagtgcccagggctctgatgagtctctcatcgcttgtaaagcctga

SEQ ID NO: 119 - DNA, 408 bp, Artificial sequence
  synthesized
tctggtggcggaggctcgggcggaggtgggtcgggtggcggcggatcaatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 120 - DNA, 2323 bp, Artificial sequence
  synthesized
atggagttggggctgagctgggttttccttgttgctatattagaaggtgtccagtgtgaggtgcagctggtggagtctgggggaggcttggtacagccgggggggtccctgagactctcctgtgcagcctctggattcaccatcagtagttacgacatgcactgggtccgccaagcaacaggaaaaggtctggagtgggtctcagttattggtcctgctggtgacacatactatccaggctccgtgaagggccgattcaccatctccagagaaaatgccaagaactccttgtatcttcaaatgaacagcctgagagccggggacacggctgtgtattactgtgcaagagaggggatggctgcccacaactactacggtatggacgtctggggccaagggaccacggtcaccgtctcctctctggtggcggaggctcgggcggaggtgggtcgggtggcggcggatcaatggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcccccgatcaccttcggccaagggacacgactggagattaaatctggtggcggaggctcgggcggaggtgggtcgggtggcggcggatcaggagtgcatccgccccaacccttttccccctcgtctcctgtgagaattccccgtcggatacgagcagcgtggccgttggctgcctcgcacaggacttccttcccgactccatcactttctcctggaaatacaagaacaactctgacatcagcagcacccggggcttcccatcagtcctgagagggggcaagtacgcagccacctcacaggtgctgctgccttccaaggacgtcatgcagggcacagacgaacacgtggtgtgcaaagtccagcaccccaacggcaacaaagaaaagaacgtgcctcttccagtgattgctgagctgcctcccaaagtgagcgtcttcgtcccaccccgcgacggcttcttcggcaacccccgcaagtccaagctcatctgccaggccacgggtttcagtccccggcagattcaggtgtcctggctgcgcgaggggaagcaggtggggtctggcgtcaccacggaccaggtgcaggctgaggccaaagagtctgggcccacgacctacaaggtgaccagcacactgaccatcaaagagagcgactggctcggccagagcatgttcacctgccgcgtggatcacaggggcctgaccttccagcagaatgcgtcctccatgtgtgtccccgatcaagacacagccatccgggtcttcgccatccccccatcctttgccagcatcttcctcaccaagtccaccaagttgacctgcctggtcacagacctgaccacctatgacagcgtgaccatctcctggacccgccagaatggcgaagctgtgaaaacccacaccaacatctccgagagccaccccaatgccactttcagcgccgtgggtgaggccagcatctgcgaggatgactggaattccggggagaggttcacgtgcaccgtgacccacacagacctgccctcgccactgaagcagaccatctcccggcccaagggggtggccctgcacaggcccgatgtctacttgctgccaccagcccgggagcagctgaacctgcgggagtcggccaccatcacgtgcctggtgacgggcttctctcccgcggacgtcttcgtgcagtggatgcagagggggcagcccttgtccccggagaagtatgtgaccagcgccccaatgcctgagccccaggccccaggccggtacttcgcccacagcatcctgaccgtgtccgaagaggaatggaacacgggggagacctacacctgcgtggtggcccatgaggccctgcccaacagggtcaccgagaggaccgtggacaagtccaccgagggggaggtgagcgccgacgaggagggctttgagaacctgtgggccaccgcctccaccttcatcgtcctcttcctcctgagcctcttctacagtaccaccgtcaccttgttcaaggtgaaatga

(19) United States
(12) Patent Application Publication
(10) Pub. No.: US 2023/0175009 A1
(43) Pub. Date: Jun. 8, 2023

(54) IMMUNOLOGICALLY COMPATIBLE AND REVERSIBLE UNIVERSAL PLURIPOTENT STEM CELL AND APPLICATION THEREOF

(71) Applicants: Linli Wang, Guangzhou, Guangdong (CN); Future Homo Sapiens Institute of Regenerative Medicine Co., Ltd., Guangzhou, Guangdong (CN)

(72) Inventors: Linli WANG, Guangzhou, Guangdong (CN); Yuehua CHEN, Guangzhou, Guangdong (CN); Jianguo YANG, Guangzhou, Guangdong (CN); Jian MO, Guangzhou, Guangdong (CN)

(21) Appl. No.: 17/922,291

(22) PCT Filed: Aug. 13, 2021
(86) PCT No.: PCT/CN2021/112388

§ 371 Date: Oct. 28, 2022

(30) Foreign Application Priority Data
Aug. 18, 2020 (CN) 202010831988.1
Sep. 24, 2020 (CN) 202011016839.6

(51) Int. Cl. C12N 15/85; C12N 5/0735; C12N 5/074; C12N 15/113; A61K 35/545

(52) U.S. Cl. CPC C12N 15/85; C12N 5/0606; C12N 5/0696; C12N 15/113; A61K 35/545; C12N 2510/00; C12N 2830/006; C12N 2310/141; C12N 2310/14

(57) ABSTRACT

Provided is an immunologically compatible and reversible universal pluripotent stem cell or a derivative thereof. An inducible gene expression system and the expression sequence of at least one immunologically compatible molecule are introduced into the genome of the pluripotent stem cell or the derivative thereof. The immunologically compatible molecule is used to regulate the expression of immune response-related genes in the pluripotent stem cell or the derivative thereof. The expression of the immunologically compatible molecule is regulated by the inducible gene expression system. The described method can increase the immunological compatibility between a transplant and a recipient, and can simultaneously reversibly restore the antigen-presenting abilities of transplant cells.

### TECHNICAL FIELD

The present disclosure belongs to the technical field of genetic engineering, and in particular relates to an immunologically compatible and reversible universal pluripotent stem cell and an application thereof.

### BACKGROUND

Stem cells are a class of “seed” cells that have the ability to self-renew and differentiate into specific functional somatic cells, have the potential to regenerate into various tissues and organs and the human body, and play a central and irreplaceable role in major biological activities such as immune response, aging, and tumorigenesis. According to the degree of stem cell characteristics, stem cells are mainly divided into totipotent stem cells, pluripotent stem cells (PSCs) and adult stem cells.

Among them, pluripotent stem cells (PSCs) have almost unlimited self-renewal capacity and the potential to develop and differentiate into organs, tissues and cells of all germ layers in an embryo under normal developmental conditions. Typical PSCs mainly include embryonic stem cells (ESCs), embryonic germ cells (EGCs), embryonic carcinoma cells (ECCs), and induced pluripotent stem cells (iPSCs), etc., and such cells have very profound and broad application prospects due to their powerful functions and can pass ethical restrictions to a certain extent.

Due to the huge application prospects of PSCs, industry-university-research work such as PSC bank construction is increasingly developing. However, the conception or establishment of either autologous iPSC cell banks or immunomatched PSC cell banks requires enormous financial, material and human resources. The molecular and immunological basis of organ, tissue or cell transplantations of allogeneic donors and recipients is mainly based on the matching of the classical major histocompatibility complexes MHC-I and MHC-II (also known as HLA-I and HLA-II in humans). As of June 2019, more than 20,000 alleles of the HLA system have been identified and named, wherein the numbers of only the classical HLA-A, B, and C alleles have respectively exceeded 5,000. The number of possible random combinations of these classical HLA-I/II alleles will be astronomical, and with the discovery of new alleles, the number of combinations increases, which brings great obstacles to tissue matching and donor selection before transplantation of organ, tissue, and cell, and also brings huge difficulties to the construction of population-covering immunomatched PSC cell banks.

Therefore, the construction of allogeneic immunologically compatible universal PSCs is imminent. In recent years, many reports have indicated that by knocking out genes such as B2M and CIITA, the expression of HLA-I and HLA-II on the cell surface or the genes themselves becomes absent. Thereby the cells can develop immune tolerance or escape T/B cell-specific immune responses, and immunologically compatible universal PSCs are generated, which lays an important foundation for the wider application of universal PSC-derived cells, tissues, and organs. It has also been reported that cells over-express CTLA4-Ig and PD-L1 to inhibit allogeneic immune rejection. Recently, it has also been reported that by knocking in CD47 while knocking out B2M and CIITA, cells develop immune tolerance or escape from innate immune responses by NK cells, etc., in addition to obtaining escape from specific immune responses, such that the cells have more comprehensive and stronger immunological compatibility properties. However, these solutions either have incomplete immunological compatibility and still have allogeneic immune rejections through other routes; or completely eliminate allogeneic immune rejection responses, but at the same time cause the cells of the donor-derived transplant to lose the antigen-presenting ability, which poses a great risk of diseases such as tumorigenicity and viral infection to the recipient.

In this regard, it has also been reported that, retaining HLA-C while knocking out HLA-A and HLA-B or together with CIITA, without directly knocking out B2M, and 12 HLA-C immunomatched antigens covering more than 90% of the human population are constructed. Such that the cells of a transplant retain a certain degree of antigen-presenting function, and at the same time, the innate immune response of NK cells can be inhibited through HLA-C. However, with regard to such cells, first of all, antigen types presented by HLA-I are reduced by more than two-thirds, the integrity of antigens that can be presented is greatly and irreversibly reduced, thereby causing highly biased presentation of antigens in various tumors, viruses and other diseases and retaining a considerable degree of risk of diseases such as tumorigenesis and viral infections remains, in addition, the risk of diseases is even higher when CIITA is also knocked out; secondly, the 12 high-frequency immunomatched HLA-C antigens are highly ethnically diverse, according to our verification and calculation, the proportion can be only 70% in some regions, in addition, and there has been no authoritative HLA data display from large sample sizes in China, India and other populous countries, so there is still a huge matching vacancy challenge for using the prepared universal PSCs; and thirdly, this method involves repeated gene-editing efforts many times. Based on at least two rounds of single-cell isolation and culture for each gene editing, the entire process requires at least six or more rounds of single-cell isolation and culture. Because of multiple off-target gene editing or chromatin instability, or due to passage and proliferation of large numbers of single cells, these processes inevitably and with great probability would cause various unpredictable mutations in cells, thereby leading to various problems such as carcinogenesis and metabolic diseases. Thus, such immunological compatibility solutions are also expedient measures in the “transition period”, and there are still many problems that have not been better resolved.

In addition, some people have designed suicide genes to induce killing after donor tissue and cells become diseased. The consequences thereof are severe tissue necrosis, cytokine storms and other unpredictable disease risk problems, and another big challenge is that after such designed cells are killed, no more suitable donor cells, tissues and organs will exist.

### SUMMARY

In view of the deficiencies in the prior art, the present disclosure designs a set of immunologically compatible and reversible universal pluripotent stem cell strategies, which are applicable to PSCs and derivatives derived from the PSCs. Such PSCs do not undergo allogeneic immune rejection when cellular immunological compatibility is required, and can reactivate specific immunity and innate immunity when an immune response is required to eliminate tumors or viruses. In addition, this strategy can even induce donor cells, tissues or organs to develop recipient tolerance under the condition of a low concentration of mismatched HLA. Thereby the universal transplant cells, tissues and organs possess both functionality and safety, and the donor cells, tissues or organs can be selectively rather than completely killed, which greatly promotes the extensive clinical application and immune tolerance research of immunologically compatible PSCs and derivatives derived from the PSCs.

### Technical Solution Used by the Disclosure

Provided is a pluripotent stem cell or a derivative thereof, wherein an inducible gene expression system and an expression sequence of at least one immunologically compatible molecule are introduced into the genome of the pluripotent stem cell or the derivative thereof;

the immunologically compatible molecule is used to regulate the expression of immune response-related genes in the pluripotent stem cell or the derivative thereof; and

the expression of the immunologically compatible molecule is regulated by the inducible gene expression system.

The inducible gene expression system is regulated by an exogenous inducer; and the on and off state of the inducible gene expression system is controlled by adjusting the added amount, duration of action, and type of the exogenous inducer so as to control the expression amount of the expression sequence of the immunologically compatible molecule.

Preferably, the inducible gene expression system includes at least one selected from the group consisting of the Tet-Off system and dimer-induced expression system.

Where the inducible gene expression system used is the Tet-Off system, the expression of the immunologically compatible molecule in the cell or the derivative thereof can be controlled by adding the exogenous inducer Doxycycline (Dox). It is even possible to stimulate the body by adjusting the added amount of Dox, which enables the cell or the derivative thereof to gradually express low concentrations of HLA molecules, such that the body can gradually develop tolerance to the transplanted cells or the derivatives thereof and finally achieve a stable tolerance. Typically, Dox is added in an amount of 0-100 μM.

Where the inducible gene expression system used is a dimer-induced expression system, the expression of the immunologically compatible molecule in the cell or the derivative thereof can be controlled by adding the exogenous inducer rapamycin (or an analog thereof). It is even possible to stimulate the body by adjusting the added amount of rapamycin (or an analog thereof), which enables the cell or the derivative thereof to gradually express low concentrations of HLA molecules, such that the body can gradually develop tolerance to the transplanted cells or the derivatives thereof and finally achieve a stable tolerance. Typically, rapamycin (or an analog thereof) is added at an amount of 0-1000 nM.

The immune response-related genes include:

1. Major histocompatibility complex genes, including:

(1) classical HLAs, including HLA-A, HLA-B, HLA-C, HLA-DRA, HLA-DRB1, HLA-DRB3, HLA-DRB4, HLA-DRB5, HLA-DQA1, HLA-DQB1, HLA-DPA1, and HLA-DPB1, wherein class I molecules (A, B, and C) are distributed on the surface of all nucleated cells, while class II molecules (DR, DQ, and DP) are only expressed on the surface of some specific cells in lymphoid tissues, such as professional antigen-presenting cells (including B cells, macrophages, and dendritic cells), thymic epithelial cells, and activated T cells. Major histocompatibility antigens (MHC antigens) are transplantation antigens that can cause a strong rejection response, namely HLA molecules encoded by the HLA complex (human MHC). Actually, the difference in HLA type between the donor and the recipient is the main cause of acute transplant rejection. Almost all the classical HLAs are associated with transplant rejection, and especially, class I molecules are extremely important.

(2) HLA-C is a locus of human leukocyte antigen (HLA) and is located on chromosome 6. Mature HLA-C protein forms a heterodimer with β2-microglobulin and is anchored on the cell surface as a receptor. There are three HLA-C alleles with frequency >10%: C*01:02, C*07:02 and C*03:04, and the frequencies of these three genes are as high as 50% or more. In addition, there are 12 HLA-C alleles with frequency >1%, and the cumulative frequency thereof is 90% or more. HLA-C antigens are classical HLA class I molecules, which not only present endogenous polypeptides to CD8+ T cells to induce specific immune responses, but also act as ligands for killer immunoglobulin-like receptors (KIRs) to regulate the killing function of natural killer cells (NK cells) and affect the immune function of the body.

In the present disclosure, by finding out the top 30 homozygous individuals with the highest proportion of HLA matching in the human population and 12 homozygotes with HLA-C allele frequency >1%, immunologically compatible and reversible universal pluripotent stem cells are prepared, which can well match most of the human population. In the 12 homozygous HLA-C individuals, we can use gene knock-in technology to knock in, at a genomic safe locus, HLA-A and HLA-B knockdown sequences of which expression can be inducibly turned off, and the sequences can safely express HLA-A and HLA-B knockdown molecules (e.g., knockdown molecules such as shRNA-miRNA and shRNA). This can retain part of the immune effect while achieving the immunological compatibility effect of HLA I molecules. When the transplant becomes diseased, the expression of HLA-A and HLA-B knockdown molecules can be inducibly turned off by an inducer, thereby reversibly enabling the re-expression of HLA-A and HLA-B molecules on the cell surface. Thereby, by recognizing mismatched HLA-A and HLA-B molecules, or by antigen-presentation of mutated molecules crosswise through HLA-A and HLA-B molecules, the immune system of the recipient enables the recipient to eliminate the diseased transplant, and the clinical safety of such universal pluripotent stem cells and derivatives thereof is improved and the value thereof in clinical applications is greatly expanded

2. Major histocompatibility complex-related genes, including B2M or CIITA:

(1) Taking B2M (i.e., β2 microglobulin) as an example, the gene named B2M (beta microglobulin) constitutes a light chain part (β chain) of HLA class I molecules. β2-m promotes the transport of the entire HLA molecule from the endoplasmic reticulum to the surface of the cell membrane and can also maintain the structural stability of the entire HLA molecule during the expression of the HLA class I molecule on the cell surface, and is a subunit necessary for surface expression, assembly and stability of HLA molecule. Many studies have reported that after the B2M gene is knocked out from a cell, HLA class I molecules are no longer expressed on the cell surface, and allogeneic HLA mismatching immune responses associated with the HLA class I molecules disappear accordingly; in addition, for HLA I-unmatched cell, tissue or organ transplants (referred to as “transplants”), the recipient will develop immunological compatibility/tolerance to the transplantation unmatched HLA I, thus achieving the aim of the construction of immunologically compatible pluripotent stem cells or derivatives derived from the pluripotent stem cells. However, after complete knockout of B2M, the cell completely loses the ability to present antigens via HLA class I molecules, and there is no way to deal with various lesions (such as canceration) in cells, tissues or organs that may occur after transplantation, which greatly affects the application safety and further clinical applications.

(2) CIITA: CIITA, i.e., the gene of class II major histocompatibility complex transactivator, is a key gene known for class II HLA gene transcription, is constitutively expressed in antigen-presenting cells, and may also be expressed in other cells under IFN-γ induction. Cells mainly regulate the expression level of HLA class II genes by regulating the expression of CIITA, thus regulating the intensity of immune response. As a co-activating molecule, CIITA is recruited to the vicinity of a promoter of the HLA class II gene, and CIITA participates in the regulation of the gene expression and transcription. The expression level of CIITA is positively correlated with the expression level of HLA II. Therefore, CIITA is considered to be the most critical regulatory molecule for HLA class II gene expression.

3. Negative costimulatory molecules on the T cell surface, including CTLA4, PD-1 or BTLA.

The immunologically compatible molecules include any one or more of:

1. negative costimulatory molecules on the antigen-presenting cell surface;

2. activating antibodies against “negative costimulatory molecules on the T cell surface” located on the transplanted cell surface (that is, “negative costimulatory molecules on the T cell surface” is located on the transplanted cell surface), wherein the “negative costimulatory molecules on the T cell surface” include at least one selected from the group consisting of CTLA4, PD-1 and BTLA;

3. stimulating ligands against “negative costimulatory molecules on the T cell surface” located on the transplanted cell surface (that is, “negative costimulatory molecules on the T cell surface” is located on the transplanted cell surface);

4. immune tolerance-related genes;

5. HLA-C class molecules;

6. shRNAs and/or shRNA-miRs for major histocompatibility complex genes; and

7. shRNAs and/or shRNA-miRs for major histocompatibility complex-related genes.

Preferably, the immunologically compatible molecules include any one or more of:

1. The antigen-presenting cell surface negative costimulatory molecules include at least one selected from the group consisting of PD-L1, Siglec-15, B7-H4 and B7-H5, wherein:

(1) PD-L1: Programmed cell death 1 ligand 1 (PD-L1), also known as cluster of differentiation 274 surface antigen (cluster of differentiation 274, CD274) or B7 homolog (B7 homolog 1, B7-H1), is a protein in humans, encoded by the CD274 gene. Programmed cell death 1 ligand 1, a type 1 transmembrane protein of 40 kDa in size, is believed to be involved in the suppression of the immune system in certain special circumstances (e.g., pregnancy, tissue transplantation, autoimmune diseases, and certain diseases such as hepatitis). Under normal circumstances, the immune system responds to foreign antigens that accumulate in the lymph nodes or spleen, and triggers antigen-specific cytotoxic T cells (CD8+ T cell proliferation). In turn, the binding of programmed cell death receptor-1 (PD-1) with programmed cell death 1 ligand 1 (PD-L1) can transmit an inhibitory signal to reduce the proliferation of CD8+ T cells in the lymph nodes, make them unable to recognize cancer cells, cause reduced proliferation or apoptosis of T cells themselves, and effectively disarm the immune response in the body, and as a result, cancer cells can grow recklessly. In addition, PD-1 can also control the accumulation of antigen-specific T cells in lymph nodes by regulating the Bcl-2 gene.

(2) Siglec-15: Siglec-15 is a member of the sialic acid-binding Ig-like lectins (Siglecs) family and is expressed in M2 macrophages, myeloid cells (MDSCs), dendritic cells, B cells and osteoclasts. Siglec-15 is a type I membrane protein with very typical and conserved structural features. As a novel T cell activity regulator, Siglec-15 can inhibit the specific immune response of T cells by regulating the growth of T cells, and blocking the immunosuppressive effect of Siglec-15 can restore/improve the anti-tumor immune response in the body.

(3) B7H4 (B7-H4, also known as B7s1 and B7x) is a member of the B7 family. B7H4 protein is composed of a signal peptide region, a pair of immunoglobulin V and C outer segments, a transmembrane domain and a cytoplasmic domain. It is a type I transmembrane glycoprotein and is riveted to the cell membrane via GPI. In addition, the cross-linking of H4 through GPI facilitates access to MHC molecules and such a spatial structure facilitates the mediation of negative costimulatory effects. B7H4 is expressed in professional APCs and is widely distributed through non-lymphoid tissues. It can negatively regulate the immune response of T cells by inhibiting T cell proliferation, cytokine production and cell cycle progression.

(4) B7-H5 (also known as VISTA, Gi24, PD-1H or Dies1) is a transmembrane protein of about 55-65 KD and is a member of the immunoglobulin superfamily. The extracellular segment of B7-H5 consists of only one IgV domain. B7-H5 is mainly expressed on the surface of mature myeloid APC cells, including macrophages, mature BMDCs, neutrophils and CD11c DC cells and activated Treg cells. B7-H5 expressed on the surface of both APC cells and T cells inhibit T cell growth, arrests the cell cycle and down-regulates cytokine production. Therefore, the negative molecule B7-H5 may be a new target for transplantation immunity.

2. The activating antibodies against the “negative costimulatory molecules on the T cell surface” located on the transplanted cell surface include the membrane antibody anti-BTLA:

BTLA is an immunosuppressive receptor and is a type I transmembrane glycoprotein. The protein structure thereof is similar to CTLA-4 and PD-1 and comprises an extracellular domain, a transmembrane domain and a cytoplasmic domain. The cytoplasmic domain of BTLA comprises three tyrosine residues linked to an immunoreceptor tyrosine-based inhibition motif (ITIM), and can bind and activate tyrosinase SHP-1 and SHP-2 after phosphorylation. Another tyrosine residue is predicted to be a Crb2 recruitment site, it can also link a p85 subunit of PI3K, and direct BTLA costimulatory immunomodulation via the PI3K pathway. A ligand for BTLA is HVEM, and HVEM mainly performs a positive regulatory function, interacts with LIGHT, promotes the proliferation of T and B cells and the production of Ig, activates NK cells via NK cell receptors, and makes them secrete GM-CSF and IFN-γ. The interaction of BTLA and HVEM transmits an inhibitory signal to down-regulate the immune response of lymphocytes. BTLA and HVEM mainly regulate the function of T cells and APCs through their dynamic expression on the cell surface. In the primary and secondary immune responses of CD4⁺ T cells and the secondary response of CD8⁺ T cells, BTLA cross-links TCR to inhibit T cell activation. The binding of BTLA to the ligand not only inhibits T cell proliferation and down-regulates the T cell activation marker CD25, but also inhibits the production of IFN-γ, IL-2, IL-4, IL-10, etc.

3. The stimulating ligands against the “negative costimulatory molecules on the T cell surface” located on the transplanted cell surface include at least one selected from the group consisting of PD-L1, Siglec-15, B7-H4 and B7-H5.

4. The immune tolerance-related genes include at least one selected from the group consisting of CD47 and HLA-G, wherein:

(1) CD47: CD47 is an integrin-associated protein (TAP), which is an adhesion molecule member of the immunoglobulin superfamily. CD47 can inhibit the phagocytosis of target cells by phagocytes by binding to its ligand (leukocyte inhibitory receptor signal regulatory protein, SIRPα). In this way, CD47 can block and inhibit the activity of almost all innate immune responses of macrophages, NK cells, etc., thereby allowing cells to escape or tolerate innate immunity and trigger negative regulatory signals such as tumor immunity, transplantation immunity, and pregnancy immunity. In addition, CD47 can also inhibit the activity of T cells through its negative costimulatory molecules, thereby inhibiting specific immune responses, and developing immunological compatibility/tolerance. The construction of an immunologically compatible pluripotent stem cell or a derivative derived from the pluripotent stem cell is achieved.

(2) HLA-G: HLA-G is a non-classical HLA class I molecule. Compared with the classical HLA class I molecules, HLA-G has 3 characteristics: (1) a low polymorphism level; (2) in vivo distribution limited to specific tissues; and (3) the formation of four membrane-bound and two soluble HLA-G isoforms due to changes in the splice sites between introns and exons. Many research results in the past few years have shown that HLA-G is an immune tolerance molecule, and the expression of HLA-G at the maternal-fetal interface plays an important role in maternal-fetal immune tolerance and the maintenance of normal pregnancy. HLA-G can play an immunosuppressive effect by directly binding to various inhibitory receptors, and killer cell immunoglobulin-like receptors that have been confirmed to have the ability to bind HLA-G molecules include: ILT2/CD85j/LILRB1, ILT4/CD58d/LILRB2, and the killer cell inhibitory receptor KIR2DL4/CD158d. These receptors can be expressed on different immune cells. By binding to the above three receptors, HLA-G molecules can effectively inhibit the proliferation of CD4+ T cells and cause loss of function, inhibit the killing function of NK cells, and inhibit the antigen presentation and cytokine secretion functions of dendritic cells, etc.

5. The HLA-C class molecules include HLA-C multiple alleles with a proportion of more than 90% in total in the human population, or fusion protein genes composed of the HLA-C multiple alleles with a proportion of more than 90% and B2M.

6. The target sequences of the shRNAs and/or shRNA-miRs for the major histocompatibility complex genes are SEQ ID NO. 14 to SEQ ID NO. 23.

7. The target sequences of the shRNAs and/or shRNA-miRs for the major histocompatibility complex-related genes are SEQ ID NO. 1 to SEQ ID NO. 13.

As another preferred solution of the present disclosure, an shRNA and/or miRNA processor complex-related gene and/or an anti-interferon effector molecule are further knocked into the genome of the pluripotent stem cell or the derivative thereof.

The shRNA and/or miRNA processor complex-related gene includes at least one selected from the group consisting of Dhrosha, Ago1, Ago2, Dicer1, Exportin-5, TRBP (TARBP2), PACT (PRKRA) and DGCR8;

and the anti-interferon effector molecule is shRNAs and/or shRNA-miR for at least one selected from the group consisting of PKR, 2-5As, IRF-3 and IRF-7.

Preferably, the target sequences of the shRNAs and/or shRNA-miRs for PKR, 2-5As, IRF-3 or IRF-7 are SEQ ID NO. 31 to SEQ ID NO. 90.

Preferably, the general backbone sequence of the above shRNAs and/or shRNA-miRs for the major histocompatibility complex genes, major histocompatibility complex-related genes, and PKR, 2-5As, IRF-3 or IRF-7 is as follows:

(1) General backbone sequence of shRNA: comprising, in sequence from 5′ to 3′, an shRNA target sequence, a stem-loop sequence, and a reverse complement sequence of the shRNA target sequence, wherein the length of the loop sequence is 3-9 bases.

For example, 5′-N₁ . . . N₂₁-TTCAAGAGA (SEQ ID NO. 24)-N₂₂ . . . N₄₂-3′

Wherein:

a. N represents the base A or T or G or C;

b. the sequence N1 . . . N21 is the same as the shRNA target sequence;

c. the sequence N22 . . . N42 is reverse complementary to the sequence N1 . . . N21;

d. this framework sequence does not include promoters and terminators, and promoters and terminators are additionally required to regulate the expression of this framework sequence through inducible expression system elements; and

e. shRNA sequence: the sequence N1 . . . N21 and the sequence N22 . . . N42 in the general backbone sequence of shRNA are replaced with the shRNA target sequence of the corresponding gene.

(2) General backbone sequence of shRNA-miR: obtained by replacing a target sequence in microRNA-30 or microRNA-155 with the shRNA-miR target sequence.

For example,

5′-GAGGCTTCAGTACTTTACAGAATCGTTGCCTGCACATCTTGGAAACACTTG CTGGGATTACTTCTTCAGGTTAACCCAACAGAAGGCTAAAGAAGGTATATTGCTGT TGACAGTGAGCG (SEQ ID NO. 25)-M₁N₁ . . . N₂₁-TAGTGAAGCCACAGATGTA (SEQ ID NO. 26)-N₂₂ . . . N₄₂M₂-TGCCTACTGCCTCGGACTTCAAGGGGCTACTTTAGGAGCA ATTATCTTGTTTACTAAAACTGAATACCTTGCTATCTCTTTGATACATTTTTACAAAG CTGAATTAAAATGGTATAAAT (SEQ ID NO. 27)-3′

or

5′-GCATACACAAACATTTCTTTCTCTCTTGCAGGTGGCACAAACCAGGAAGGG GAAATCTGTGGTTTAAATTCTTTATGCCTCATCCTCTGAGTGCTGAAGGCTTGCTGT AGGCTGTATGC (SEQ ID NO. 28)-M₁N₁ . . . N₂₁-TAGTGAAGCCACAGATGTA (SEQ ID NO. 29)-N₂₂ . . . N₄₂M₂-GTGTATGATGCCTGTTACTAGCATTCACATGGAACAAATTGCT GCCGTGGGAGGATGACAAAGAAGCATGAGTCACCCTGCTGGATAAACTTAGACTT CAGGCTTTATCATTTTTCAAT (SEQ ID NO. 30)-3′

wherein:

a. the base M represents the base A or C, and N represents the base A or T or G or C;

b. the base M1 is complementary to the base M2; and the sequence N1 . . . N21 is reverse complementary to the sequence N22 . . . N42;

c. if N1 is the base G, then M1 is the base A; otherwise, M1 is the base C;

d. the sequence N1 . . . N21 is the same as the shRNA target sequence;

e. this framework sequence does not include promoters and terminators, and promoters and terminators are additionally required to regulate the expression of this framework sequence through inducible expression system elements; and

f. shRNA-miR sequence:

The sequences N1 . . . N21 and N22 . . . N42 in the backbone sequence of shRNA-miR are replaced with the shRNA target sequence of the corresponding gene.

Preferably, the inducible gene expression system, the expression sequence of the immunologically compatible molecule, the shRNA and/or miRNA processor complex-related gene, and the anti-interferon effector molecule are introduced by means of viral vector interference, non-viral vector transfection or gene editing.

Preferably, the gene editing method includes gene knock-in.

Preferably, introduction loci for the inducible gene expression system, the expression sequence of the immunologically compatible molecule, the shRNA and/or miRNA processor complex-related gene, and the anti-interferon effector molecule are genomic safe loci.

Preferably, the genomic safe loci include at least one selected from the group consisting of the AAVS1 safe locus, the eGSH safe locus, and the H11 safe locus.

With regard to the above-mentioned pluripotent stem cell or derivative thereof,

the pluripotent stem cell includes an embryonic stem cell, an embryonic germ cell, an embryonic carcinoma cell, or an induced pluripotent stem cell;

the derivative includes a pluripotent stem cell-derived three-germ-layer-derived organ, tissue or cell; and the pluripotent stem cell-derived three-germ-layer-derived cell includes a mesenchymal stem cell, a neural stem cell or a neural progenitor cell, or other adult stem cell.

Provided is a method for preparing an immunologically compatible and reversible universal pluripotent stem cell and a derivative thereof, involving introducing an inducible gene expression system and an expression sequence of at least one immunologically compatible molecule into the genome of the pluripotent stem cell or the derivative thereof, wherein the immunologically compatible molecule is used to regulate the expression of immune response-related genes in the pluripotent stem cell or the derivative thereof.

Provided is a use of the above-mentioned immunologically compatible and reversible universal pluripotent stem cell and derivative thereof in the preparation of a product for cellular therapy.

Provided is a use of the above-mentioned immunologically compatible and reversible universal pluripotent stem cell or derivative thereof in the preparation of a product for organ transplantation.

Provided is a use of the above-mentioned immunologically compatible and reversible universal pluripotent stem cell and derivative thereof in the construction of a universal PSC cell bank.

Provided is a use of the above-mentioned immunologically compatible and reversible universal pluripotent stem cell or derivative thereof as a gene-drug carrier. This can be achieved by introducing an expression sequence of a gene-drug into the genome of the immunologically compatible and reversible universal pluripotent stem cell or derivative thereof.

### Beneficial Effects of the Disclosure

(1) In the present disclosure, by introducing an inducible gene expression system and an expression sequence of the immunologically compatible molecule into the genome of a pluripotent stem cell or a derivative thereof, an immunologically compatible and reversible universal pluripotent stem cell or a derivative thereof is obtained. The inducible gene expression system is regulated by an exogenous inducer; and the on and off state of the inducible gene expression system is controlled by adjusting the added amount, duration of action, and type of the exogenous inducer so as to control the expression amount of the expression sequence of the immunologically compatible molecule. The immunologically compatible molecule is used to regulate the expression of immune response-related genes in the pluripotent stem cell or the derivative thereof. When the immunologically compatible molecule is normally expressed, the expression of immune response-related genes in the pluripotent stem cell or the derivative thereof is inhibited or excess, so that during tissue transplantation, the allogeneic immune rejection response can be eliminated or reduced, thereby improving the immunological compatibility between the transplant and the recipient. In addition, when the transplant becomes diseased, the expression of the immunologically compatible molecule can be inducibly turned off by an exogenous inducer, which reversibly enables re-expression of HLA class I molecules on the cell surface and restores the antigen-presenting capacity of the transplant cells. Thereby the immune system of the recipient enables the recipient to eliminate the diseased transplant by recognizing mismatched HLA class I molecules or by antigen-presentation of mutated molecules crosswise by HLA class I molecules (antigen presentation/recognition between classical incompatible HLAs), which improves the clinical safety of such universal pluripotent stem cells or derivatives thereof and greatly expands the value thereof in clinical applications.

(2) As a preferred solution, the present disclosure uses gene knock-in technology to knock-in an inducible gene expression system and an expression sequence of an immunologically compatible molecule at a genomic safe locus (safe harbor) (e.g., loci such as AAVS1), whereby the immunologically compatible molecule can cause inhibited expression or over-expression of immune response-related genes in the pluripotent stem cell or the derivative thereof. When the immunologically compatible molecule is normally expressed, the expression of immune response-related genes in the pluripotent stem cell or the derivative thereof is inhibited or excess, so that during tissue transplantation, the allogeneic immune rejection response can be eliminated or reduced, thereby improving the immunological compatibility between the transplant and the recipient. In addition, when the transplant becomes diseased, the expression of the immunologically compatible molecule in the transplant cells can be inducibly turned off by an exogenous inducer, which reversibly enables re-expression of HLA class I molecules on the cell surface and restores the antigen-presenting capacity of the transplant cells. Thereby the immune system of the recipient enables the recipient to eliminate the diseased transplant by recognizing mismatched HLA class I molecules or by antigen-presentation of mutated molecules crosswise by HLA class I molecules (antigen presentation/recognition between classical incompatible HLAs), which improves the clinical safety of such universal pluripotent stem cells or derivatives thereof and greatly expands the value thereof in clinical applications.

(3) In addition, it is also possible to stimulate the recipient by adjusting the added amount and duration of action of the exogenous inducer, which enables the transplant to gradually express low concentrations of HLA molecules, such that the recipient gradually develops tolerance to the transplant and finally achieves a stable tolerance. In this case, even if the transplant cells express mismatched HLA class I molecules on the surface, they are also compatible with the recipient's immune system, so that after the expression of the immunologically compatible molecule in the transplant cell is inducibly turned off, the recipient's immune system can, on the one hand, re-identify cells (cells are those presented by HLA class I molecule) with genetic mutations in the transplant, and eliminate diseased cells; on the other hand, the part in which no mutation occurs is not eliminated by the recipient's immune system due to training by the above inducer to develop tolerance to allogeneic HLA class I molecules. Therefore, the recipient's immune system only eliminates transplants with harmful mutations and retains transplants with normal functions, and after the harmful transplants are eliminated, it can switch to a mode of silencing HLA class I molecules on the transplant cell surface. After the recipient has developed complete tolerance, the transplant immune tolerance procedure mediated by the exogenous inducer may also allow implantation of a transplant in which surface expression of HLA class I molecules is turned on or off without induction or through induction by other means.

In the present disclosure, the universal pluripotent stem cells or derivative thereof will be enabled to adapt to the immune system of the recipient to the greatest extent possible, to be safe and controllable to the greatest extent possible, and to engage with a still further safe treatment to the greatest extent possible.

### BRIEF DESCRIPTION OF DRAWINGS

FIG. 1 shows an “AAVS1 KI Vector (shRNA, shRNA-miR, Gene, Neo)” KI Donor plasmid map.

FIG. 2 shows an “AAVS1 KI Vector (shRNA, shRNA-miR, Gene, Puro)” KI Donor plasmid map.

FIG. 3 shows a Cas9(D10A) plasmid map.

FIG. 4 shows a gRNA clone AAVS1-1 plasmid map.

FIG. 5 shows a gRNA clone AAVS1-2 plasmid map.

FIG. 6 shows a gRNA clone eGSH-1 plasmid map.

FIG. 7 shows a gRNA clone eGSH-2 plasmid map.

FIG. 8 shows the cell-specific ⁵¹Cr release rate (T cell activity) of hPSC-derived EB sphere immunologically compatible cells in Example 2.

FIG. 9 shows the cell-specific ⁵¹Cr release rate (T cell activity) of hPSC-derived EB sphere immunologically compatible cells after a Dox treatment in Example 3.

FIG. 10 shows the cell-specific ⁵¹Cr release rate (NK cell activity) of hPSC-derived EB sphere immunologically compatible cells in Example 4.

FIG. 11 shows the cell-specific ⁵¹Cr release rate (NK cell activity) of hPSC-derived EB sphere immunologically compatible cells after a Dox treatment in Example 5.

FIG. 12 shows the percentage of CFSE⁺PI⁺ cells in hPSC-derived EB sphere cells in Example 6.

FIG. 13 shows the percentage of CFSE⁺PI⁺ cells in hPSC-derived EB sphere cells after a Dox treatment in Example 7.

FIG. 14 shows the NK cell cytotoxicity value in Example 8.

FIG. 15 shows the NK cell cytotoxicity value after a Dox treatment in Example 9.

FIG. 16 shows ⁵¹Cr natural release rate (T cell activity) of hPSC-MSC, NSC and EB immunologically compatible cells in Example 10.

FIG. 17 shows ⁵¹Cr natural release rate (NK cell activity) of hPSC-MSC, NSC and EB immunologically compatible cells in Example 11.

FIG. 18 shows the percentage of CFSE⁺PI⁺ cells in hPSC, MSC, NSC and EB immunologically compatible cells in Example 12.

FIG. 19 shows the NK cell cytotoxicity value in Example 13.

FIG. 20 shows the cell-specific ⁵¹Cr release rate (T cell activity) of hPSC, MSC, NSC and EB immunologically compatible cells in Example 14.

FIG. 21 shows the MTT values of the hPSC, MSC, NSC and EB immunologically compatible cells in Example 15.

### DETAILED DESCRIPTION

In order to understand the technical content of the present disclosure more clearly, the following examples are specifically given for detailed description in conjunction with the accompanying drawings. It should be understood that these examples are only used to describe the present disclosure, rather than limiting the scope of the present disclosure.

Experimental methods in which no specific conditions are indicated in the following examples are usually carried out under conventional conditions, for example, the conditions described in Sambrook et al., Molecular Cloning: A Laboratory Manual (New York: Cold Spring Harbor Laboratory Press, 1989), or in accordance with the conditions recommended by the manufacturer. Various common chemical reagents used in the examples are all commercially available products.

### Example 1 Method for Preparing Immunologically Compatible and Reversible Universal Pluripotent Stem Cells and Derivatives Thereof

Provided was a method for preparing an immunologically compatible and reversible universal pluripotent stem cell and a derivative thereof, involving introducing an inducible gene expression system and an expression sequence of at least one immunologically compatible molecule into the genome of the pluripotent stem cell or the derivative thereof, wherein the immunologically compatible molecule was used to regulate the expression of immune response-related genes in the pluripotent stem cell or the derivative thereof; and the expression of the immunologically compatible molecule was regulated by the inducible gene expression system.

When carrying out immunological compatibility modification, the modification could be carried out on hPSCs first, and after the modification was completed, they were then differentiated into pluripotent stem cell derivatives for use; alternatively, immunological compatibility modification could be carried out after hPSCs were differentiated into pluripotent stem cell derivatives.

In the method:

I. Pluripotent Stem Cells and Derivatives Thereof

Pluripotent stem cells were selected from embryonic stem cells (ESCs), induced pluripotent stem cells (iPSCs) and other forms of pluripotent stem cells, such as hPSCs-MSCs, NSCs, and EB cells. Among them:

iPSCs: pE3.1-OG—KS and pE3.1-L-Myc—hmiR302 cluster were electrotransfected into somatic cells by using our established third-generation efficient and safe episomal-iPSC induction system (6F/BM1-4C), and cultured in RM1 for 2 days, in BioCISO-BM1 with 2 μM Parnate for 2 days, in BioCISO-BM1 with 2 μM Parnate, 0.25 mM sodium butyrate, 3 μM CHIR99021 and 0.5 μM PD03254901 for 2 days, and in the stem cell medium BioCISO until approximately 17 days, iPSC clones were picked, and the picked iPSC clones were purified, digested, and passaged to obtain stable iPSCs. For the specific construction method, see: Stem Cell Res Ther. 2017 Nov. 2; 8(1): 245.

hPSCs-MSCs: iPSCs were cultured in a stem cell medium (BioCISO, containing 10 μM TGFβ inhibitor SB431542) for 25 days during which digestion and the passage were carried out at 80-90 confluence (2 mg/mL Dispase digestion), the cells were passaged 1:3 into a Matrigel-coated culture plate and then into an ESC-MSC medium (knockout DMEM medium, containing 10% KSR, NEAA, double antibody, glutamine, β-mercaptoethanol, 10 ng/mL bFGF and SB-431542), wherein the medium was changed every day, the passage was carried out at 80-90 confluence (1:3 passage), and the cells were cultured continuously for 20 days. For the specific construction method, see: Proc Natl Acad Sci USA. 2015; 112(2): 530-535.

NSCs: iPSCs were cultured in an induction medium (knockout DMEM medium, containing 10% KSR, TGF-β inhibitor, and BMP4 inhibitor) for 14 days, and rosette-shaped nerve cells were picked and cultured in a low-adherence culture plate, wherein the medium used was DMEM/F12 (containing 1% N2, Invitrogen) and Neurobasal medium (containing 2% B27, Invitrogen) at a ratio of 1:1 and further contained 20 ng/ml bFGF and 20 ng/ml EGF, and for digestion, Accutase was used for digestion and passage. For the specific construction method, see: FASEB J. 2014; 28(11): 4642-4656.

EB cells: iPSCs with a confluence of 95% were digested with BioC-PDE1 for 6 min and then scraped into a mass by a mechanical scraping passage method, cell mass settling occurred, and the settled cell mass was transferred to a low-adherence culture plate and cultured using BioCISO-EB1 for 7 days during which the medium was changed every other day. After 7 days, the cells were transferred to a Matrigel-coated culture plate to continue adherent culture with BioCISO, and after 7 days, embryoid bodies (EBs) with a structure of inner, middle and outer germ layers could be obtained. For the specific construction method, see: Stem Cell Res Ther. 2017 Nov. 2; 8(1): 245.

The derivative included pluripotent stem cell-derived three-germ-layer-derived organs, tissues or cells; and the pluripotent stem cell-derived three-germ-layer-derived cells included mesenchymal stem cells, neural stem or progenitor cells, or other adult stem cells.

II. Genomic Safe Locus (Safe Harbor)

It could be selected from the AAVS1 safe locus, the eGSH safe locus, or other safe loci:

(1) The AAVS1 Safe Locus

the AAVS1 locus (alias the “PPP1R2C locus”), located on chromosome 19 of the human genome, is a validated “safe harbor” locus that could ensure the intended function of the transferred DNA fragment. This locus is an open chromosomal structure, which could ensure that the transferred gene could be transcribed normally, and the insertion of an exogenous fragment of interest at this locus has no known side effects on cells.

(2) The eGSH Safe Locus

the eGSH safe locus, located on chromosome 1 of the human genome, is another documented validated “safe harbor” locus that could ensure the intended function of the transferred DNA fragment.

(3) Other Safe Loci

the H11 safe locus (also called Hipp11), located on human chromosome 22, is a locus between the two genes Eif4enif1 and Drg1. The locus was discovered and named by Simon Hippenmeyer in 2010. Since the H11 locus is located between the two genes, the risk of affecting the expression of endogenous genes after the insertion of an exogenous gene is small. The H11 locus is verified to be an intergenic safe transcription activation region, a new “safe harbor” locus other than the AAVS1 locus and the eGSH locus.

III. Inducible Gene Expression System

1. The Tet-Off System

In the absence of doxycycline, the tTA protein continued to act on the tet promoter, resulting in continuous gene expression. This system was useful where a transgene was required to be maintained in a state of continuous expression. When doxycycline was added, doxycycline could change the structure of the tTA protein, making it impossible to bind to the promoter, thereby reducing the level of gene expression driven thereby. To keep this system in an “off” state, continuous addition of doxycycline was necessary.

In the present disclosure, the tet-Off system and one or more immunologically compatible molecule sequences were knocked into the genomic safe locus of pluripotent stem cells, whereby the expression of the immunologically compatible molecule could be accurately turned on or off depending on whether to add doxycycline or not, such that the expression of major histocompatibility complex-related genes in the pluripotent stem cell or the derivative thereof could be reversibly regulated.

2. Dimer Turn-Off Expression System

Dimer-mediated gene expression regulation system: There were many approaches to chemically regulate the transcription of target genes, and the most common one was regulation by an allosteric modulator that affected the activity of transcription factors. One such approach was to use a dimerization inducer or a dimer to reconstitute active transcription factors on an inactive fusion protein. The most commonly used system was the natural product rapamycin or a biologically inactive analog as a dimerization agent. Rapamycin (or an analog thereof) had a high affinity with the homoplasmic protein FKBP12 (an FKBP protein that bound to FK506) and a large serine-threonine protein kinase called FRAP [FRBP-rapamycin-related protein, or mTOR (target of rapamycin in mammalian)], and also had the function of combining with these two proteins, so that these two proteins were brought together as a heterodimer. To regulate the transcription of a target gene, a DNA binding domain was fused to one or more FKBP domains, and a transcription repression domain was fused to the 93 amino acid position of FRAP, called FRB. As such, it was sufficient to bind the FKBP-rapamycin complex. The two fusion proteins could dimerize only in the presence of rapamycin (or an analog thereof). Thus, the transcription of a gene with a binding site to the DNA-binding domain was inhibited.

IV. Immunologically Compatible Molecule

The immunologically compatible molecule could regulate the expression of immune response-related genes in a pluripotent stem cell or a derivative thereof. The types and sequences of specific immunologically compatible molecules were shown in Table 1:

TABLE 1
Immunologically compatible molecules

| | Immunologically | | The function of |
|---|---|---|---|
| | compatible | Specific | immunologically |
| | molecule | immunologically | compatible |
| No. | classification | compatible molecule | molecule |
| 1 | shRNA of major | B2M (beta-2- | Knocking down the |
| | histocompatibility | microglobulin) | expression of B2M. |
| | complex gene- | shRNA | |
| | related | | |
| 2 | gene | CIITA shRNA | Knocking down the |
| | | | expression of CIITA. |
| 3 | shRNA-miR of | B2M (beta-2- | Knocking down the |
| | major complex | microglobulin) | expression of B2M. |
| | gene-related gene | shRNA-miR | |
| 4 | histocompatibility | CIITA shRNA-miR | Knocking down the |
| | | | expression of CIITA. |
| 5 | shRNA of major | HLA-A shRNA | Knocking down the |
| | complex gene | | expression of HLA-A. |
| 6 | histocompatibility | HLA-B shRNA | Knocking down the |
| | | | expression of HLA-B. |
| 7 | shRNA-miR of | HLA-A shRNA-miR | Knocking down the |
| | major | | |
| | histocompatibility | | expression of HLA-A. |
| 8 | complex gene | HLA-B shRNA-miR | Knocking down the |
| | | | expression of HLA-B. |
| 9 | Major | HLA-C retained | HLA-C was a major |
| | histocompatibility | (preferably retained | histocompatibility |
| | complex gene | homozygous HLA- | antigen that could |
| | HLA-C | C, preferably | cause a strong |
| | homozygote | retained | rejection response. |
| | | homozygous HLA-C | There were 12 HLA- |
| | | from 30 HLA-C | C alleles with |
| | | strains that covered | frequency >1%, and |
| | | more than 90% of | the cumulative |
| | | the human | frequency was 90% or |
| | | population). | more. Therefore, it |
| | | The sequences of | could be retained, |
| | | HLA-C | and immunological |
| | | homozygotes were | compatibility could be |
| | | shown in SEQ ID | achieved as long as |
| | | NO. 107 to SEQ ID | matching was |
| | | NO. 118, | satisfied. |
| | | respectively. | |
| 10 | Major | HLA-C-B2M fusion | Direct expression of |
| | histocompatibility | protein (preferably | fully functional HLA- |
| | complex gene | retained | C molecules. |
| | HLA-C-B2M | homozygous HLA- | Immunological |
| | fusion gene | C, preferably | compatibility could be |
| | homozygote | retained | achieved as long as |
| | | homozygous | matching was |
| | | HLA-C from 30 | satisfied. |
| | | HLA-C strains that | |
| | | covered more than | |
| | | 90% of the human | |
| | | population) | |
| 11 | Immune tolerance- | CD47 | Blocking and |
| | related genes | (Accession number: | inhibiting the activity |
| | | NM_198793.2) | of almost all the |
| | | | innate immune |
| | | | responses of |
| | | | macrophages, NK |
| | | | cells, etc., and |
| | | | inhibiting T cell |
| | | | activity through the |
| | | | effect of its negative |
| | | | costimulatory |
| | | | molecules. |
| 12 | | HLA-G | Exerting |
| | | (Accession number: | immunosuppressive |
| | | NM_002127) | effects by directly |
| | | | binding to various |
| | | | inhibitory receptors. |
| 13 | Stimulating | PD-L1 | The binding of PD-1 |
| | ligands against | (Accession number: | with PD-L1 could |
| | “negative | NM_001314029) | transmit an inhibitory |
| | costimulatory | | signal to reduce the |
| | molecules on the T | | proliferation of CD8+ |
| | cell surface” | | T cells in the lymph |
| | located on the | | nodes and cause |
| | transplanted cell | | reduced proliferation |
| | surface | | or apoptosis of T cells |
| | | | themselves, |
| | | | effectively disarming |
| | | | the immune response |
| | | | in the body. |
| 14 | | Siglec-15 | Siglec-15 inhibited |
| | | (Accession number: | the specific immune |
| | | NM_213602) | response of T cells by |
| | | | regulating the growth |
| | | | of T cells, and |
| | | | blocking the |
| | | | immunosuppressive |
| | | | effect of Siglec-15 |
| | | | could restore/improve |
| | | | the anti-tumor |
| | | | immune response in |
| | | | the body. |
| 15 | | B7H4 | B7H4 could |
| | | (Accession number: | negatively regulate |
| | | NM_001253850) | the immune response |
| | | | of T cells by |
| | | | inhibiting T cell |
| | | | proliferation, cytokine |
| | | | production and cell |
| | | | cycle progression. |
| 16 | | B7H5 | B7-H5 inhibited T |
| | | (Accession number: | cell growth, arrested |
| | | NM_001282559) | the cell cycle and |
| | | | down-regulated |
| | | | cytokine production. |
| 17 | Activating | BTLA activating | Blocking the binding |
| | antibodies against | antibody | of BTLA with |
| | “negative | (SEQ ID NO. 120) | HVEM, hindering the |
| | costimulatory | | interaction between |
| | molecules on the T | | HVEM and LIGHT, |
| | cell surface” | | and thereby limiting |
| | located on the | | the proliferation of T |
| | transplanted cell | | and B cells and the |
| | surface | | production of Ig, etc. |

The major histocompatibility complex gene HLA-C homozygote in Table 1 was at least one of HLA-C*01:02 (SEQ ID NO. 107), HLA-C*02:02 (SEQ ID NO. 108), HLA-C*03:03 (SEQ ID NO. 109), HLA-C*03:04 (SEQ ID NO. 110), HLA-C*04:01 (SEQ ID NO. 111), HLA-C*05:01 (SEQ ID NO. 112), HLA-C*06:02 (SEQ ID NO. 113), HLA-C*07:01 (SEQ ID NO. 114), HLA-C*07:02 (SEQ ID NO. 115), HLA-C*08:01 (SEQ ID NO. 116), HLA-C*12:02 (SEQ ID NO. 117) and HLA-C*16:01 (SEQ ID NO. 118).

The sequence of the HLA-C-B2M fusion gene in Table 1 was shown below:

(SEQ ID NO. 119)
N-TCTGGTGGCGGAGGCTCGGGCGGAGGTGGGTCGGGTGGCGGCGGATC
AATGTCTCGCTCCGTGGCCTTAGCTGTGCTCGCGCTACTCTCTCTTTCT
GGCCTGGAGGCTATCCAGCGTACTCCAAAGATTCAGGTTTACTCACGTC
ATCCAGCAGAGAATGGAAAGTCAAATTTCCTGAATTGCTATGTGTCTGG
GTTTCATCCATCCGACATTGAAGTTGACTTACTGAAGAATGGAGAGAGA
ATTGAAAAAGTGGAGCATTCAGACTTGTCTTTCAGCAAGGACTGGTCTT
TCTATCTCTTGTACTACACTGAATTCACCCCCACTGAAAAAGATGAGTA
TGCCTGCCGTGTGAACCATGTGACTTTGTCACAGCCCAAGATAGTTAAG
TGGGATCGAGACATGTAA

Wherein N represented at least one of the HLA-C sequences, shown in SEQ ID NO. 107 to SEQ ID NO. 118 respectively.

The target sequences of the above shRNA or shRNA-miR for the immunologically compatible molecule were shown in Table 2:

TABLE 2
Target sequence of shRNA or shRNA-miR

| | | | | |
|---|---|---|---|---|
| No. | Start | Target sequence (DNA) | Region | Sequence number |

B2M

| | | | | |
|---|---|---|---|---|
| 1 | 634 | GGGAGCAGAGAATTCTCTTAT | UTR3 | SEQ ID NO. 1 |
| 2 | 635 | GGAGCAGAGAATTCTCTTATC | UTR3 | SEQ ID NO. 2 |
| 3 | 636 | GAGCAGAGAATTCTCTTATCC | UTR3 | SEQ ID NO. 3 |

CIITA

| | | | | |
|---|---|---|---|---|
| 1 | 231 | GCTACCTGGAGCTTCTTAACA | ORF | SEQ ID NO. 4 |
| 2 | 238 | GGAGCTTCTTAACAGCGATGC | ORF | SEQ ID NO. 5 |
| 3 | 879 | GGGTCTCCAGTATATTCATCT | ORF | SEQ ID NO. 6 |
| 4 | 2656 | GCCTCCTGATGCACATGTACT | ORF | SEQ ID NO. 7 |
| 5 | 2917 | GGAAGACCTGGGAAAGCTTGT | ORF | SEQ ID NO. 8 |
| 6 | 3276 | GGCTAAGCTTGTACAATAACT | ORF | SEQ ID NO. 9 |
| 7 | 3688 | GCGGAATGAACCACATCTTGC | UTR3 | SEQ ID NO. 10 |
| 8 | 3801 | GGCCTTCTCTGAAGGACATTG | UTR3 | SEQ ID NO. 11 |
| 9 | 4354 | GGACTCAATGCACTGACATTG | UTR3 | SEQ ID NO. 12 |
| 10 | 4578 | GGTACCCACTGCTCTGGTTAT | UTR3 | SEQ ID NO. 13 |

HLA-A

| | | | | |
|---|---|---|---|---|
| 1 | 96 | GCTCCCACTCCATGAGGTATT | ORF | SEQ ID NO. 14 |
| 2 | 111 | GGTATTTCTTCACATCCGTGT | ORF | SEQ ID NO. 15 |
| 3 | 279 | AGGAGACACGGAATGTGAAGG | ORF | SEQ ID NO. 16 |
| 4 | 1164 | CCCTTCCCTTTGTGACTTGAA | UTR3 | SEQ ID NO. 17 |

HLA-B

| | | | | |
|---|---|---|---|---|
| 1 | 95 | GCTCCCACTCCATGAGGTATT | ORF | SEQ ID NO. 18 |
| 2 | 110 | GGTATTTCTACACCTCCGTGT | ORF | SEQ ID NO. 19 |
| 3 | 273 | GGACCGGAACACACAGATCTA | ORF | SEQ ID NO. 20 |
| 4 | 275 | ACCGGAACACACAGATCTACA | ORF | SEQ ID NO. 21 |
| 5 | 278 | GGAACACACAGATCTACAAGG | ORF | SEQ ID NO. 22 |
| 6 | 279 | GAACACACAGATCTACAAGGC | ORF | SEQ ID NO. 23 |

The sequence of the above shRNA or shRNA-miR for the immunologically compatible molecule was as follows:

1. shRNA Sequence

(1) General backbone sequence of shRNA: comprising, in sequence from 5′ to 3′, an shRNA target sequence, a stem-loop sequence, and a reverse complement sequence of the shRNA target sequence. Specifically, as follows,

5′-N₁ . . . N₂₁TTCAAGAGA N₂₂ . . . N₄₂-3′

wherein:

a. N represented the base A or T or G or C;

b. the sequence N1 . . . N21 was the same as the shRNA target sequence;

c. the sequence N22 . . . N42 was reverse complementary to the sequence N1 . . . N21;

d. this backbone sequence did not include promoters and terminators, and promoters and terminators were additionally required to regulate the expression of this backbone sequence through inducible expression system elements; and

(2) shRNA sequence: the sequence N1 . . . N21 and the sequence N22 . . . N42 in the general backbone sequence of shRNA were replaced with the shRNA target sequence of the corresponding gene.

2. shRNA-miR Sequence

(1) General backbone sequence of shRNA-miR: obtained by replacing a target sequence in microRNA-30 with the shRNA-miR target sequence. Specifically, as follows,

5′-GAGGCTTCAGTACTTTACAGAATCGTTGCCTGCACATCTTGGAAACA
CTTGCTGGGATTACTTCTTCAGGTTAACCCAACAGAAGGCTAAAGAAGGT
ATATTGCTGTTGACAGTGAGCGM₁N₁ . . . N₂₁TAGTGAAGCCACAGA
TGTAN₂₂ . . . N₄₂M₂TGCCTACTGCCTCGGACTTCAAGGGGCTACTT
TAGGAGCAATTATCTTGTTTACTAAAACTGAATACCTTGCTATCTCTTTG
ATACATTTTTACAAAGCTGAATTAAAATGGTATAAAT-3′

wherein:

a. the base M represented the base A or C, and N represented the base A or T or G or C;

b. the base M1 was complementary to the base M2; and the sequence N1 . . . N21 was reverse complementary to the sequence N22 . . . N42;

c. if N1 was the base G, then M1 was the base A; otherwise, M1 was the base C;

d. the sequence N1 . . . N21 was the same as the shRNA target sequence; and

e. this backbone sequence did not include promoters and terminators, and the promoters and terminators were additionally required to regulate the expression of this backbone sequence through inducible expression system elements.

(2) shRNA-miR Sequence:

The sequences N1 . . . N21 and N22 . . . N42 in the backbone sequence of shRNA-miR were replaced with the shRNA target sequence of the corresponding gene.

In the immunologically compatible molecule, knock-in schemes in Tables 3 to 6 below, the shRNA or shRNA-miR immunologically compatible molecule sequences of each experimental group were all shRNA or shRNA-miR immunologically compatible molecules constructed using the target sequences 1 in Table 2. Those skilled in the art can understand that shRNA or shRNA-miR immunologically compatible molecules constructed using other target sequences can also achieve the technical effects of the present disclosure and all fall within the scope of the claims of the present disclosure.

V. Knock-In Scheme of the Immunologically Compatible Molecule

1. Experimental Scheme and Grouping

The experimental schemes for knocking in one or more immunologically compatible molecules into genomic safe loci of pluripotent stem cells were shown in Tables 3-6, wherein Aa1-D3 represented experimental groups, and the sign “+” represented the knocked-in immunologically compatible molecule.

TABLE 3
Knock-in scheme I of the immunologically compatible molecule

| Immunologically | | | | | | | | | | | |
|---|---|---|---|---|---|---|---|---|---|---|---|
| compatible | Experimental group | | | | | | | | | | |
| molecule | Aa1 | Aa2 | Ab1 | Ab2 | Ac1 | Ac2 | Ad1 | Ad2 | Ae1 | Ae2 | Af1 |
| B2M (beta-2- | + | | | | + | | | | | | |
| microglobulin) | | | | | | | | | | | |
| shRNA | | | | | | | | | | | |
| CIITA shRNA | | | + | | + | | | | | | |
| B2M (beta-2- | | + | | | | + | | | | | |
| microglobulin) | | | | | | | | | | | |
| shRNA-miR | | | | | | | | | | | |
| CIITA shRNA- | | | | + | | + | | | | | |
| miR | | | | | | | | | | | |
| HLA-A shRNA | | | | | | | + | | | | + |
| HLA-B shRNA | | | | | | | | | + | | + |
| HLA-A shRNA- | | | | | | | | + | | | |
| miR | | | | | | | | | | | |
| HLA-B shRNA- | | | | | | | | | | + | |
| miR | | | | | | | | | | | |
| HLA-C retained | | | | | | | | | | | |
| (preferably | | | | | | | | | | | |
| retained | | | | | | | | | | | |
| homozygous | | | | | | | | | | | |
| HLA-C, | | | | | | | | | | | |
| preferably | | | | | | | | | | | |
| retained | | | | | | | | | | | |
| homozygous | | | | | | | | | | | |
| HLC-C from 30 | | | | | | | | | | | |
| HLA-C strains | | | | | | | | | | | |
| that covered | | | | | | | | | | | |
| more than 90% | | | | | | | | | | | |
| of the human | | | | | | | | | | | |
| population) | | | | | | | | | | | |
| HLA-C-B2M | | | | | | | | | | | |
| fusion protein | | | | | | | | | | | |
| (preferably | | | | | | | | | | | |
| retained | | | | | | | | | | | |
| homozygous | | | | | | | | | | | |
| HLA-C, | | | | | | | | | | | |
| preferably | | | | | | | | | | | |
| retained | | | | | | | | | | | |
| homozygous | | | | | | | | | | | |
| HLC-C from 30 | | | | | | | | | | | |
| HLA-C strains | | | | | | | | | | | |
| that covered | | | | | | | | | | | |
| more than 90% | | | | | | | | | | | |
| of the human | | | | | | | | | | | |
| population) | | | | | | | | | | | |
| CD47 | | | | | | | | | | | |
| HLA-G | | | | | | | | | | | |
| PD-L1 | | | | | | | | | | | |
| Siglec-15 | | | | | | | | | | | |
| B7H4 | | | | | | | | | | | |
| B7H5 | | | | | | | | | | | |
| BTLA activating | | | | | | | | | | | |
| antibody | | | | | | | | | | | |

TABLE 4
Knock-in scheme II of immunologically compatible molecule

| Specific | | | | | | | | | | | |
|---|---|---|---|---|---|---|---|---|---|---|---|
| immunologically | | | | | | | | | | | |
| compatible | Experimental group | | | | | | | | | | |
| molecule | Af2 | Ag1 | Ag2 | Ah | Ai1 | Ai2 | Ai3 | Ai4 | Ai5 | Ai6 | B1 |
| B2M (beta-2- | | | | | | | | | | | |
| microglobulin) | | | | | | | | | | | |
| shRNA | | | | | | | | | | | |
| CIITA shRNA | | | | | | | | | | | |
| B2M (beta-2- | | | + (3′UTR) | | + | + | + | | | | + |
| microglobulin) | | | | | | | | | | | |
| shRNA-miR | | | | | | | | | | | |
| CIITA shRNA-miR | | | | + | + | + | + | + | + | + | + |
| HLA-A shRNA | | | | | | | | | | | |
| HLA-B shRNA | | | | | | | | | | | |
| HLA-A shRNA- | + | + | | + | | | | + | + | + | |
| miR | | | | | | | | | | | |
| HLA-B shRNA- | + | + | | + | | | | + | + | + | |
| miR | | | | | | | | | | | |
| HLA-C retained | | + | | + | | | | + | + | + | |
| (preferably retained | | | | | | | | | | | |
| homozygous HLA- | | | | | | | | | | | |
| C, preferably | | | | | | | | | | | |
| retained | | | | | | | | | | | |
| homozygous HLC- | | | | | | | | | | | |
| C from 30 HLA-C | | | | | | | | | | | |
| strains that covered | | | | | | | | | | | |
| more than 90% of | | | | | | | | | | | |
| the human | | | | | | | | | | | |
| population). | | | | | | | | | | | |
| HLA-C*01:02 | | | | | | | | | | | |
| (SEQ ID NO. 107) | | | | | | | | | | | |
| was taken as an | | | | | | | | | | | |
| example here. | | | | | | | | | | | |
| HLA-C-B2M | | | + | | | | | | | | |
| fusion protein | | | | | | | | | | | |
| (preferably retained | | | | | | | | | | | |
| homozygous HLA- | | | | | | | | | | | |
| C, preferably | | | | | | | | | | | |
| retained | | | | | | | | | | | |
| homozygous HLC- | | | | | | | | | | | |
| C from 30 HLA-C | | | | | | | | | | | |
| strains that covered | | | | | | | | | | | |
| more than 90% of | | | | | | | | | | | |
| the human | | | | | | | | | | | |
| population). | | | | | | | | | | | |
| HLA-C*01:02- | | | | | | | | | | | |
| B2M fusion gene | | | | | | | | | | | |
| was taken as an | | | | | | | | | | | |
| example here. | | | | | | | | | | | |
| CD47 | | | | | + | | + | + | | + | + |
| HLA-G | | | | | | + | + | | + | + | + |
| PD-L1 | | | | | | | | | | | + |
| Siglec-15 | | | | | | | | | | | |
| B7H4 | | | | | | | | | | | |
| B7H5 | | | | | | | | | | | |
| BTLA activating | | | | | | | | | | | |
| antibody | | | | | | | | | | | |

TABLE 5
Knock-in scheme III of immunologically compatible molecule

| Specific immunologically | | | | | | | | | | | |
|---|---|---|---|---|---|---|---|---|---|---|---|
| compatible molecule | B2 | B3 | B4 | B5 | C1 | C2 | C3 | C4 | C5 | C6 | C7 |
| B2M (beta-2-microglobulin) | | | | | | | | | | | |
| shRNA | | | | | | | | | | | |
| CIITA shRNA | | | | | | | | | | | |
| B2M (beta-2-microglobulin) | + | + | + | + | + | + | + | + | + | + | + |
| shRNA-miR | | | | | | | | | | | |
| CIITA shRNA-miR | + | + | + | + | + | + | + | + | + | + | + |
| HLA-A shRNA | | | | | | | | | | | |
| HLA-B shRNA | | | | | | | | | | | |
| HLA-A shRNA-miR | | | | | | | | | | | |
| HLA-B shRNA-miR | | | | | | | | | | | |
| HLA-C retained (preferably | | | | | | | | | | | |
| retained homozygous HLA-C, | | | | | | | | | | | |
| preferably retained homozygous | | | | | | | | | | | |
| HLC-C from 30 HLA-C strains | | | | | | | | | | | |
| that covered more than 90% of | | | | | | | | | | | |
| the human population) | | | | | | | | | | | |
| HLA-C-B2M fusion protein | | | | | | | | | | | |
| (preferably retained homozygous | | | | | | | | | | | |
| HLA-C, preferably retained | | | | | | | | | | | |
| homozygous HLC-C from 30 | | | | | | | | | | | |
| HLA-C strains that covered more | | | | | | | | | | | |
| than 90% of the human | | | | | | | | | | | |
| population) | | | | | | | | | | | |
| CD47 | + | + | + | + | + | + | + | + | + | + | + |
| HLA-G | + | + | + | + | + | + | + | + | + | + | + |
| PD-L1 | | | | | + | | + | + | + | + | + |
| Siglec-15 | + | | | | + | + | | + | + | + | + |
| B7H4 | | + | | | + | + | + | | + | | + |
| B7H5 | | | + | | + | + | + | + | | | + |
| BTLA activating antibody | | | | + | + | + | + | + | + | + | |

TABLE 6
Knock-in scheme IV of immunologically compatible molecule

| Specific immunologically compatible molecule | D1 | D2 | D3 |
|---|---|---|---|
| B2M (beta-2-microglobulin) shRNA | | | |
| CIITA shRNA | | | |
| B2M (beta-2-microglobulin) shRNA-miR | + | + | |
| CIITA shRNA-miR | + | + | + |
| HLA-A shRNA | | | |
| HLA-B shRNA | | | |
| HLA-A shRNA-miR | | | + |
| HLA-B shRNA-miR | | | + |
| HLA-C retained (preferably retained homozygous HLA-C, | | | + |
| preferably retained homozygous HLA-C from 30 HLA-C | | | |
| strains that covered more than 90% of the human | | | |
| population) HLA-C*02:02 (SEQ ID NO. 108) was taken as | | | |
| an example here. | | | |
| HLA-C-B2M fusion protein (preferably retained | | | |
| homozygous HLA-C, preferably retained homozygous | | | |
| HLA-C from 30 HLA-C strains that covered more than | | | |
| 90% of the human population) | | | |
| CD47 | + | | + |
| HLA-G | | + | |
| PD-L1 | + | + | + |
| Siglec-15 | + | + | + |
| B7H4 | + | + | + |
| B7H5 | | | |
| BTLA activating antibody | + | + | + |

2. Construction of KI Plasmid in Each Group

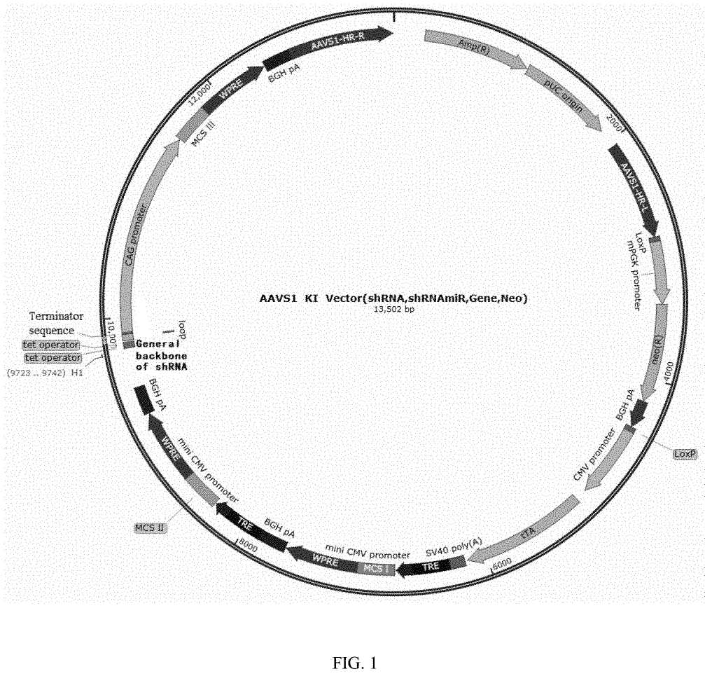

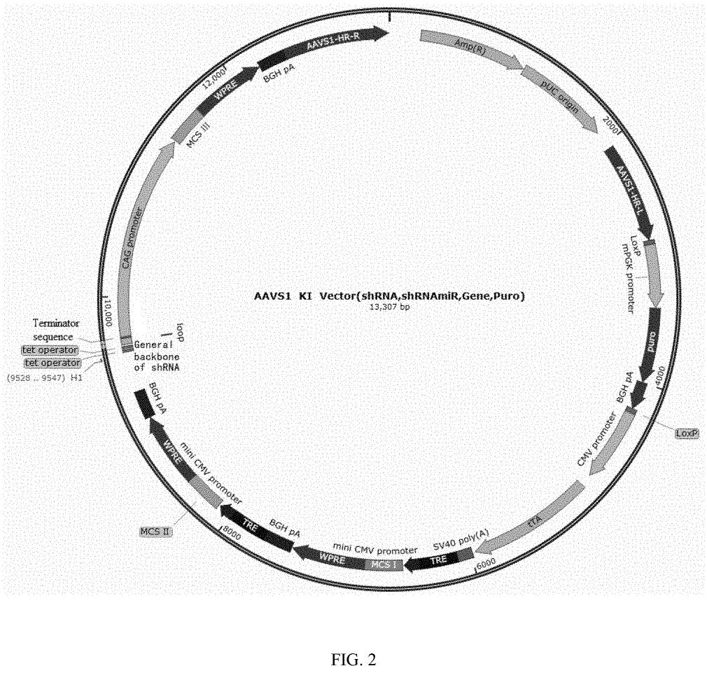

An “AAVS1 KI Vector (shRNA, shRNA-miR, Gene, Neo)” KI Donor plasmid and an “AAVS1 KI Vector (shRNA, shRNA-miR, Gene, Puro)” KI Donor plasmid were constructed. These two plasmids were collectively referred to as the “AAVS1 KI Vector” KI backbone plasmid (the same below), and the plasmid structures were shown in FIGS. 1 and 2.

Construction Method:

a. Acquisition of basic backbone of the plasmid: Primers were designed, an Amp(R)-pUC origin fragment was obtained from pUC18 (Takara, Code No. 3218) plasmid by PCR, and the product was then recovered.

b. Acquisition of recombination arms: Primers were designed, the genomic DNA of a human cell was taken as a template, AAVS1-HR-L (SEQ ID NO. 25) and AAVS1-HR-R (SEQ ID NO. 26) fragments were amplified, and the product was then recovered.

c. Acquisition of processor complex genes: mRNA was extracted from a human cell and reverse transcribed into cDNA, primers were then designed, the cDNA was used as a template to amplify the genes DROSHA and AGO2, and finally, the product was recovered.

d. Acquisition of other plasmid elements: Primers were designed, plasmids containing the plasmid elements were directly subcloned, and the product was then recovered.

e. Plasmid assembly: The various products obtained in the previous steps were ligated into a circular plasmid by overlap PCR or by recombination using a recombinase (Nanjing Vazyme Biotech, C113-01).

The immunologically compatible molecule was knocked into the “AAVS1 KI Vector” KI backbone plasmid at MCS I (multiple cloning site I) and/or MCS II (multiple cloning site II) and/or at the position of the general backbone of shRNA. The specific knock-in positions were as follows:

(1) Group Aa1: The immunologically compatible molecule “B2M shRNA” was knocked in at the “general backbone of shRNA”.

(2) Group Aa2: The immunologically compatible molecule “B2M shRNA-miR” was knocked in at “MCS I”.

(3) Group Ab1: The immunologically compatible molecule “CIITA shRNA” was knocked in at the “general backbone of shRNA”.

(4) Group Ab2: The immunologically compatible molecule “CIITA shRNA-miR” was knocked in at “MCS I”.

(5) Group Ac1: The immunologically compatible molecules “B2M shRNA” and “CIITA shRNA” were knocked in at the “general backbone of shRNA” (the 2 immunologically compatible molecules were seamlessly linked).

(6) Group Ac2: The immunologically compatible molecules “B2M shRNA-miR” and “CIITA shRNA-miR” were knocked in at “MCS I” (the 2 immunologically compatible molecules were seamlessly linked).

(7) Group Ad1: The immunologically compatible molecule “HLA-A shRNA” was knocked in at the “general backbone of shRNA”.

(8) Group Ad2: The immunologically compatible molecule “HLA-A shRNA-miR” was knocked in at “MCS I”.

(9) Group Ae1: The immunologically compatible molecule “HLA-B shRNA” was knocked in at the “general backbone of shRNA”.

(10) Group Ae2: The immunologically compatible molecule “HLA-B shRNA-miR” was knocked in at “MCS I”.

(11) Group Af1: The immunologically compatible molecules “HLA-A shRNA” and “HLA-B shRNA” were knocked in at the “general backbone of shRNA” (the 2 immunologically compatible molecules were seamlessly linked).

(12) Group Af2: The immunologically compatible molecules “HLA-A shRNA-miR” and “HLA-B shRNA-miR” were knocked in at “MCS I” (the 2 immunologically compatible molecules were seamlessly linked).

(13) Group Ag1: The immunologically compatible molecules “HLA-A shRNA-miR” and “HLA-B shRNA-miR” were knocked in at “MCS I” (the 2 immunologically compatible molecules were seamlessly linked), and the immunologically compatible molecule “HLA-C” was knocked in at “MCS II”.

(14) Group Ag2: The immunologically compatible molecule “B2M shRNA-miR 3′UTR” was knocked in at “MCS I”, and the immunologically compatible molecule “HLA-C-B2M” was knocked in at “MCS II”.

(15) Group Ah: The immunologically compatible molecules “CIITA shRNA-miR”, “HLA-A shRNA-miR” and “HLA-B shRNA-miR” were knocked in at “MCS I” (the 3 immunologically compatible molecules were seamlessly linked), and the immunologically compatible molecule “HLA-C” was knocked in at “MCS II”.

(16) Group Ai1: The immunologically compatible molecules “B2M shRNA-miR” and “CIITA shRNA-miR” (the 2 immunologically compatible molecules were seamlessly linked) were knocked in at “MCS I”, and the immunologically compatible molecule “CD47” was knocked in at “MCS II”.

(17) Group Ai2: The immunologically compatible molecules “B2M shRNA-miR” and “CIITA shRNA-miR” were knocked in at “MCS I” (the 2 immunologically compatible molecules were seamlessly linked), and the immunologically compatible molecule “HLA-G” was knocked in at “MCS II”.

(18) Group Ai3: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47” and “HLA-G” were knocked in at “MCS II” (the 2 immunologically compatible molecules were linked using EMCV IRESwt).

(19) Group Ai4: The immunologically compatible molecules “CIITA shRNA-miR”, “HLA-A shRNA-miR” and “HLA-B shRNA-miR” were knocked in at “MCS I” (the 3 immunologically compatible molecules were seamlessly linked), and the immunologically compatible molecules “HLA-C” and “CD47” were knocked in at “MCS II” (the 2 immunologically compatible molecules were linked using EMCV IRESwt).

(20) Group Ai5: The immunologically compatible molecules “CIITA shRNA-miR”, “HLA-A shRNA-miR” and “HLA-B shRNA-miR” were knocked in at “MCS I” (the 3 immunologically compatible molecules were seamlessly linked), and the immunologically compatible molecules “HLA-C” and “HLA-G” were knocked in at “MCS II” (the 2 immunologically compatible molecules were linked using EMCV IRESwt).

(21) Group Ai6: The immunologically compatible molecules “CIITA shRNA-miR”, “HLA-A shRNA-miR” and “HLA-B shRNA-miR” were knocked in at “MCS I” (the 3 immunologically compatible molecules were seamlessly linked), and the immunologically compatible molecules “HLA-C”, “CD47” and “HLA-G” were knocked in at “MCS II” (the 3 immunologically compatible molecules were linked using EMCV IRESwt).

(22) Group B1: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G” and “PD-L1” were knocked in at “MCS II” (the 3 immunologically compatible molecules were linked using EMCV IRESwt).

(23) Group B2: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G” and “Siglec-15” were knocked in at “MCS II” (the 3 immunologically compatible molecules were linked using EMCV IRESwt).

(24) Group B3: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G” and “B7H4” were knocked in at “MCS II” (the 3 immunologically compatible molecules were linked using EMCV IRESwt).

(25) Group B4: The immunologically compatible molecule “B2M shRNA-miR” and “CIITA shRNA-miR” were knocked in at “MCS I” (the 2 immunologically compatible molecules were seamlessly linked), and the immunologically compatible molecules “CD47”, “HLA-G” and “B7H5” were knocked in at “MCS II” (the 3 immunologically compatible molecules were linked using EMCV IRESwt).

(26) Group B5: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G” and “BTLA activating antibody” were knocked in at “MCS II” (the 3 immunologically compatible molecules were linked using EMCV IRESwt).

(27) Group C1: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G”, “PD-L1”, “Siglec-15”, “B7H4”, “B7H5” and “BTLA activating antibody” were knocked in at “MCS II” (the 7 immunologically compatible molecules were linked using EMCV IRESwt).

(28) Group C2: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G”, “Siglec-15”, “B7H4”, “B7H5” and “BTLA activating antibody” were knocked in at “MCS II” (the 6 immunologically compatible molecules were linked using EMCV IRESwt).

(29) Group C3: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G”, “PD-L1”, “B7H4”, “B7H5” and “BTLA activating antibody” were knocked in at “MCS II” (the 6 immunologically compatible molecules were linked using EMCV IRESwt).

(30) Group C4: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G”, “PD-L1”, “Siglec-15”, “B7H5” and “BTLA activating antibody” were knocked in at “MCS II” (the 6 immunologically compatible molecules were linked using EMCV IRESwt).

(31) Group C5: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G”, “PD-L1”, “Siglec-15”, “B7H4” and “BTLA activating antibody” were knocked in at “MCS II” (the 6 immunologically compatible molecules were linked using EMCV IRESwt).

(32) Group C6: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G”, “PD-L1”, “Siglec-15” and “BTLA activating antibody” were knocked in at “MCS II” (the 5 immunologically compatible molecules were linked using EMCV IRESwt).

(33) Group C7: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “HLA-G”, “PD-L1”, “Siglec-15”, “B7H4” and “B7H5” were knocked in at “MCS II” (the 6 immunologically compatible molecules were linked using EMCV IRESwt).

(34) Group D1: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “PD-L1”, “Siglec-15”, “B7H4” and “BTLA activating antibody” were knocked in at “MCS II” (the 5 immunologically compatible molecules were linked using EMCV IRESwt).

(35) Group D2: The immunologically compatible molecules “B2M shRNA-miR” and “CIITA shRNA-miR” were knocked in at “MCS I” (the 2 immunologically compatible molecules were seamlessly linked), and the immunologically compatible molecules “HLA-G”, “PD-L1”, “Siglec-15”, “B7H4” and “BTLA activating antibody” were knocked in at “MCS II” (the 5 immunologically compatible molecules were linked using EMCV IRESwt).

(36) Group D3: The immunologically compatible molecules “B2M shRNA-miR” and “CIITA shRNA-miR” were knocked in at “MCS I” (the 2 immunologically compatible molecules were seamlessly linked), and the immunologically compatible molecules “CD47”, “PD-L1”, “Siglec-15”, “B7H4” and “BTLA activating antibody” were knocked in at “MCS II” (the 5 immunologically compatible molecules were linked using EMCV IRESwt).

VI. shRNA/miRNA Processor Complex-Related Gene and Anti-Interferon Effector Molecule

Primary miRNA (pri-miRNA) in the nucleus was microprocessed by the complex Drosha-DGCR8, the pri-miRNA was cleaved into precursor miRNA (pre-miRNA), and at this time, a hairpin structure was formed. Next, the pre-miRNA was transported out of the nucleus via the Exportin-S-Ran-GTP complex. RNase Dicer enzyme, which bound to the double-stranded RNA-binding protein TRBP (TARBP2) in the cytoplasm, cleaved the pre-miRNA into a mature length, and at this time, the miRNA was still in a double-stranded state. Finally, it was transported into AGO2 to form RISC (RNA-induced silencing complex). Ultimately, one strand of the miRNA duplex remained in the RISC complex, while the other was expelled and rapidly degraded. As the main binding protein of Drosha, DGCR8 could bind to pri-miRNA via its two double-stranded RNA binding regions at the C-terminus and recruit and guide Drosha to cleave the pri-miRNA at the correct position to produce pre-miRNA, and the pre-miRNA was further processed and cleaved by Dicer and TRBP/PACT to form mature miRNA. The deletion or abnormal expression of DGCR8 could affect the cleaving activity of Drosha, which affects the activity of miRNA and lead to the occurrence of diseases. TRBP could recruit the Dicer complex and miRNA to form RISC Ago2.

When the present disclosure utilized gene knock-in technology to knock-in shRNA-miR expression sequences (which were directed at HLA class I molecules and HLA class II molecules and the expression of which could be inducibly turned off) at a genomic safe locus, it was preferable to simultaneously knock in shRNA and/or miRNA processing machinery the expression of which could be inducibly turned off expression, including at least one selected from the group consisting of Dhrosha (Accession number: NM_001100412), Ago1 (Accession number: NM_012199), Ago2 (Accession number: NM_001164623), Dicer1 (Accession number: NM_001195573), Exportin-5 (Accession number: NM_020750), TRBP (Accession number: NM_134323), PACT (Accession number: NM_003690) and DGCR8 (Accession number: NM_022720), such that cell functions and processing for other miRNAs were not affected.

In addition, during induced IFN production, double-stranded RNA-dependent Protein Kinase (PKR, which was a key factor in the entire cell signal transduction pathway) and 2′,5′-oligoadenylate synthetase (2-5As) were both involved, and these two enzymes were closely related to dsRNA-induced IFN production. PKR could inhibit protein synthesis by phosphorylating eukaryotic cell transcription factors, making cells stagnate in G0/G1 and G2/M phases and inducing apoptosis, while dsRNA could promote 2-5As synthesis, resulting in non-specific activation of an RNase, i.e., Rnase L, causing all mRNAs in the cells to be degraded, leading to cell death. Induction specificity for type I interferon was achieved by members of the IRF transcription factor family. In the absence of the expression of IRF-3 and IRF-7 in cells, type I interferon could not be induced and could not be secreted in many viral infection circumstances. IFN response was absent, and the recovery thereof required co-expression of the above two proteins.

The present disclosure utilized gene knock-in technology to knock-in an expression sequence of immunologically compatible molecule shRNA-miR at a genomic safe locus; preferably sequences of shRNA and/or shRNA-miR for at least one selected from the group consisting of PKR, 2-5As, IRF-3 and IRF-7 (the expression of at least one selected from the group consisting of PKR, 2-5As, IRF-3 and IRF-7 can be inducibly turned off) is simultaneously knocked in, so as to reduce dsRNA-induced interferon response and avoid cytotoxicity.

The order of the insertion positions of the shRNA/miRNA processor complex-related gene, the anti-interferon effector molecule, the immunologically compatible molecule at the genomic safe loci was not limited. They could be arranged in any order without interfering with each other or affecting the structures and functions of other genes in the genome.

The specific target sequences of anti-interferon effector molecules were shown in Table 7.

TABLE 7
Target sequences of anti-interferon effector molecules

| Interferon | | | | | |
|---|---|---|---|---|---|
| effector | | | | | |
| molecule | No. | Start | Target sequence (DNA) | Region | Sequence number |
| PKR | 1 | 1538 | GGATGGATTTGATTATGATCC | ORF | SEQ ID NO. 31 |
| | 2 | 1672 | GGACCTTGGAACAATGGATTG | ORF | SEQ ID NO. 32 |
| | 3 | 1982 | GCTAATTCTTGCTGAACTTCT | ORF | SEQ ID NO. 33 |
| | 4 | 1992 | GCTGAACTTCTTCATGTATGT | ORF | SEQ ID NO. 34 |
| | 5 | 2722 | GCCTCATCTCTTTGTTCTAAA | UTR3 | SEQ ID NO. 35 |
| | 6 | 3708 | GCTCTGGAGAAGATATATTTG | UTR3 | SEQ ID NO. 36 |
| | 7 | 4080 | GCTCTTGAGGGAACTAATAGA | UTR3 | SEQ ID NO. 37 |
| | 8 | 4144 | GGGACGGCATTAATGTATTCA | UTR3 | SEQ ID NO. 38 |
| | 9 | 4250 | GGACAAACATGCAAACTATAG | UTR3 | SEQ ID NO. 39 |
| | 10 | 4270 | GCAGCAACCAGCTACCATTCT | UTR3 | SEQ ID NO. 40 |
| 2-5 As | 1 | 49 | GCAGTTCTGTTGCCACTCTCT | UTR5 | SEQ ID NO. 41 |
| (OAS1) | 2 | 363 | GGGAGAGTTCATCCAGGAAAT | ORF | SEQ ID NO. 42 |
| | 3 | 364 | GGAGAGTTCATCCAGGAAATT | ORF | SEQ ID NO. 43 |
| | 4 | 365 | GAGAGTTCATCCAGGAAATTA | ORF | SEQ ID NO. 44 |
| | 5 | 400 | GCCTGTCAAAGAGAGAGAGCA | ORF | SEQ ID NO. 45 |
| | 6 | 471 | GCTCAGCTTCGTACTGAGTTC | ORF | SEQ ID NO. 46 |
| | 7 | 644 | GCTTCACAGAACTACAGAGAG | ORF | SEQ ID NO. 47 |
| | 8 | 884 | GCATCTACTGGACAAAGTATT | ORF | SEQ ID NO. 48 |
| | 9 | 1055 | GGCTGAATTACCCATGCTTTA | ORF | SEQ ID NO. 49 |
| | 10 | 1056 | GCTGAATTACCCATGCTTTAA | ORF | SEQ ID NO. 50 |
| 2-5 As | 1 | 138 | GGGTTGGTTTATCCAGGAATA | ORF | SEQ ID NO. 51 |
| (OAS2) | 2 | 354 | GGATCAGAAGAGAAGCCAACG | ORF | SEQ ID NO. 52 |
| | 3 | 467 | GGTTCACCATCCAGGTGTTCA | ORF | SEQ ID NO. 53 |
| | 4 | 698 | GCTCTCTTCTCTGGAACTAAC | UTR3 | SEQ ID NO. 54 |
| | 5 | 1211 | GCTAGAGTGACTCCATCTTAA | UTR3 | SEQ ID NO. 55 |
| | 6 | 1302 | GCTGACCACCAATTATAATTG | UTR3 | SEQ ID NO. 56 |
| | 7 | 1322 | GCAGAATATTTAAGGCCATAC | UTR3 | SEQ ID NO. 57 |
| | 8 | 1393 | GCCCACTTAAAGGCAGCATTA | UTR3 | SEQ ID NO. 58 |
| | 9 | 1442 | GGTCATCAATACCACTGTTAA | UTR3 | SEQ ID NO. 59 |
| | 10 | 1958 | GCATTCCTCCTTCTCCTTTCT | UTR3 | SEQ ID NO. 60 |
| 2-5 As | 1 | 620 | GGAGGAACTTTGTGAACATTC | ORF | SEQ ID NO. 61 |
| (OAS3) | 2 | 776 | GCTGTAAGAAGGATGCTTTCA | ORF | SEQ ID NO. 62 |
| | 3 | 1985 | GCTGCAGGCAGGATTGTTTCA | ORF | SEQ ID NO. 63 |
| | 4 | 2619 | GCAGTTCGAGGTCAAGTTTGA | ORF | SEQ ID NO. 64 |
| | 5 | 3852 | GCCAATTAGCTGAGAAGAATT | UTR3 | SEQ ID NO. 65 |
| | 6 | 3983 | GCAGGTTTACAGTGTATATGT | UTR3 | SEQ ID NO. 66 |
| | 7 | 5130 | GCCTACAGAGACTAGAGTAGG | UTR3 | SEQ ID NO. 67 |
| | 8 | 5478 | GCAGTTGGGTACCTTCCATTC | UTR3 | SEQ ID NO. 68 |
| | 9 | 5573 | GCAACTCAGGTGCATGATACA | UTR3 | SEQ ID NO. 69 |
| | 10 | 5993 | GCATGGCGCTGGTACGTAAAT | UTR3 | SEQ ID NO. 70 |
| IRF3 | 1 | 148 | GCCTCGAGTTTGAGAGCTA | UTR5 | SEQ ID NO. 71 |
| | 2 | 301 | AGACATTCTGGATGAGTTA | UTR5 | SEQ ID NO. 72 |
| | 3 | 43 | GGGTCTGTTACCCAAAGAA | UTR5 | SEQ ID NO. 73 |
| | 4 | 44 | GGTCTGTTACCCAAAGAAT | UTR5 | SEQ ID NO. 74 |
| | 5 | 508 | GGAAGGAAGCGGACGCTCA | ORF | SEQ ID NO. 75 |
| | 6 | 275 | GGAGGCAGTACTTCTGATA | UTR5 | SEQ ID NO. 76 |
| | 7 | 111 | CGCTCTAGAGCTCAGCTGA | UTR5 | SEQ ID NO. 77 |
| | 8 | 834 | CCACCACCTCAACCAATAA | UTR3 | SEQ ID NO. 78 |
| | 9 | 78 | ATTTCAAGAAGTCGATCAA | UTR5 | SEQ ID NO. 79 |
| | 10 | 488 | GAAGATCTGATTACCTTCA | ORF | SEQ ID NO. 80 |
| IRF-7 | 1 | 68 | GGACACTGGTTCAACACCTGT | UTR5 | SEQ ID NO. 81 |
| | 2 | 75 | GGTTCAACACCTGTGACTTCA | UTR5 | SEQ ID NO. 82 |
| | 3 | 83 | ACCTGTGACTTCATGTGTGCG | UTR5 | SEQ ID NO. 83 |
| | 4 | 1098 | GCTGGACGTGACCATCATGTA | ORF | SEQ ID NO. 84 |
| | 5 | 1101 | GGACGTGACCATCATGTACAA | ORF | SEQ ID NO. 85 |
| | 6 | 1102 | GACGTGACCATCATGTACAAG | ORF | SEQ ID NO. 86 |
| | 7 | 1103 | ACGTGACCATCATGTACAAGG | ORF | SEQ ID NO. 87 |
| | 8 | 1512 | ACGCTATACCATCTACCTGGG | ORF | SEQ ID NO. 88 |
| | 9 | 1694 | GCCTCTATGACGACATCGAGT | ORF | SEQ ID NO. 89 |
| | 10 | 1705 | GACATCGAGTGCTTCCTTATG | ORF | SEQ ID NO. 90 |

The shRNA or shRNA-miR backbone sequence of the above anti-interferon effector molecules was the same as the shRNA or shRNA-miR backbone sequence of the immunologically compatible molecules.

In the anti-interferon effector molecule knock-in schemes of Examples 14-16 below, the anti-interferon effector molecule sequences of each experimental group were all shRNAs or shRNA-miRs constructed by using target sequence 1 in Table 7. Those skilled in the art can understand that shRNA or shRNA-miR anti-interferon effector molecules constructed using other target sequences can also achieve the technical effects of the present disclosure and all fall within the scope of protection of the claims of the present disclosure.

VII. Method for Knocking Exogenous Gene Inducible Expression System (Tet-Off System or Dimer Turn-Off Expression System) and Immunologically Compatible Molecule into Genomic Safe Loci of Pluripotent Stem Cell and Test Method

(I) Construction of sgRNA

1. Plasmid

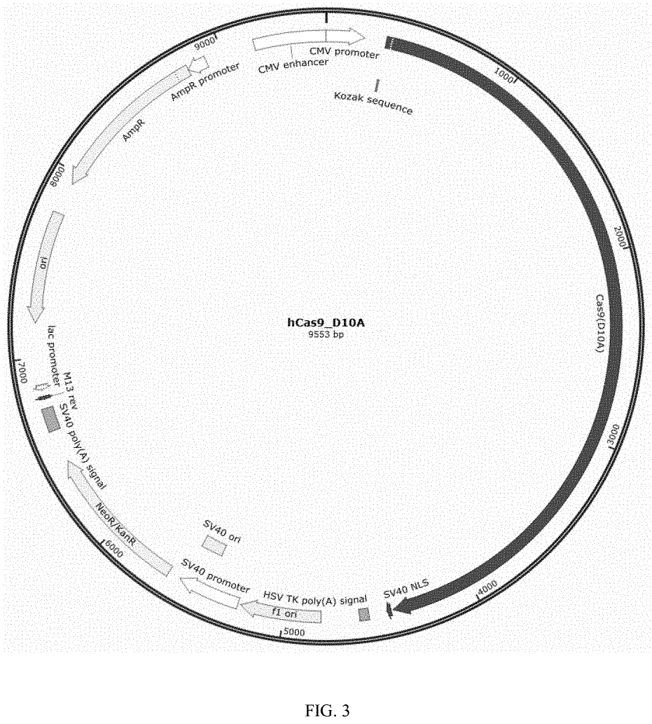

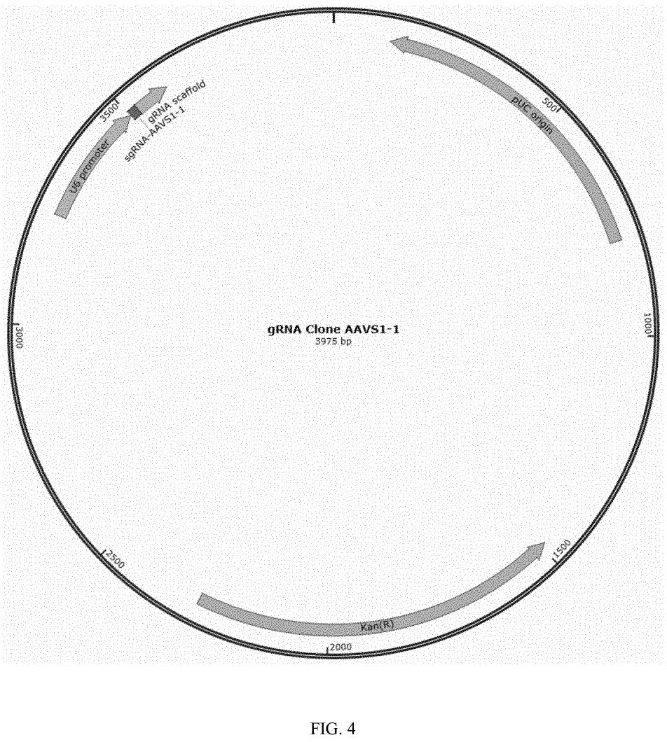

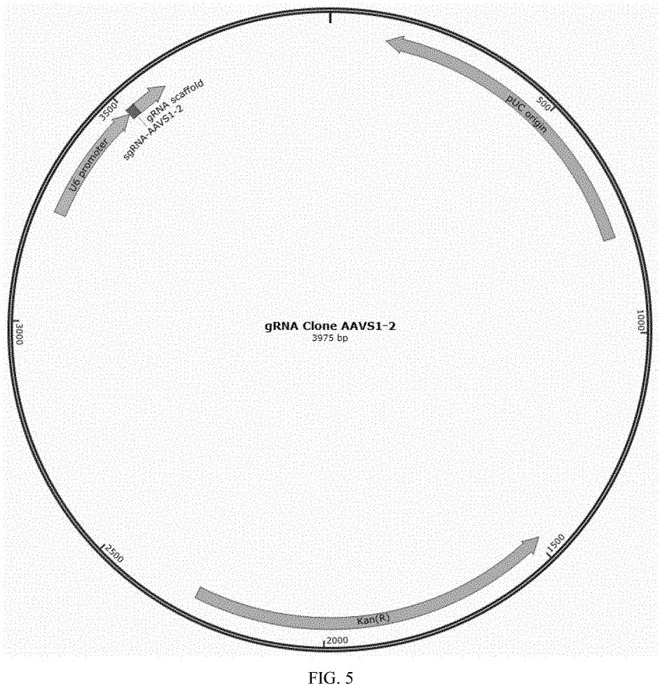

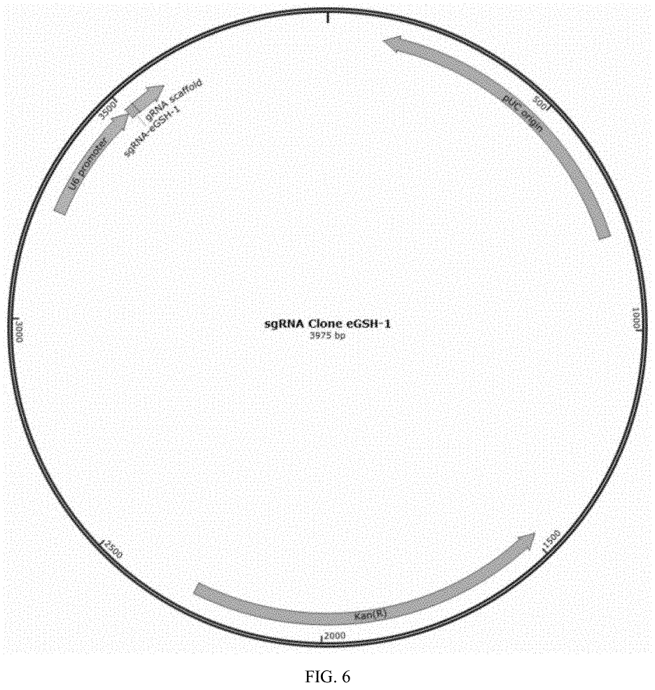

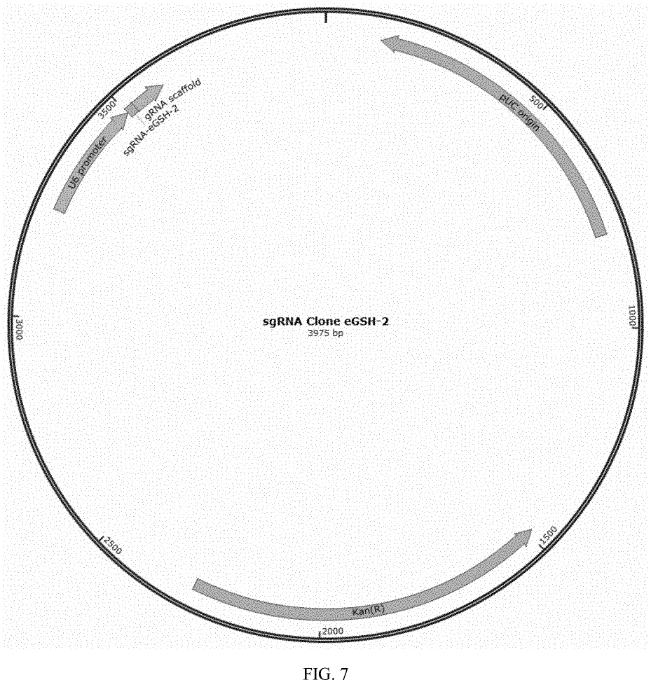

Cas9(D10A) plasmid and sgRNA plasmid were used to knock in exogenous genes. The map of the Cas9(D10A) plasmid was shown in FIG. 3, the map of the sgRNA plasmid with the AAVS1 safe locus was shown in FIGS. 4 and 5, and the map of the sgRNA plasmid with the eGSH safe locus was shown in FIGS. 6 and 7.

2. Homologous Arms

(1). The nucleotide sequences of the AAVS1 homology arms AAVS1-HR-L and AAVS1-HR-R were shown in SEQ ID NO. 91 and SEQ ID NO. 92, respectively.

(2). The nucleotide sequences of the eGSH homology arms eGSH-HR-L and eGSH-HR-R were shown in SEQ ID NO. 93 and SEQ ID NO. 94, respectively.

3. sgRNA Sequence

sgRNA-AAVS1-1:
(SEQ ID NO. 95)
5′-TATAAGGTGGTCCCAGCTCGGGG-3′;
sgRNA-AAVS1-2:
(SEQ ID NO. 96)
5′-AGGGCCGGTTAATGTGGCTCTGG-3′.
sgRNA-eGSH-1:
(SEQ ID NO. 97)
5′-GGTGGAAGCTTCATTCCAGATGG-3′;
sgRNA-eGSH-2:
(SEQ ID NO. 98)
5′-GACCTGCCTCATTAAATATCAGG-3′.

4. Plasmid Construction Method

(1) Empty sgRNA vector was digested with the restriction endonuclease BbsI and then recovered.

(2) sgRNA primers (with vector sticky ends) were synthesized.

(3) The primers were diluted with water to 10 μM, a reaction system was prepared and boiled in boiling water for 5 min, and the product was then cooled to room temperature to obtain an annealed product.

Reaction system: upstream primer: 2 μL, downstream primer: 2 μL, and water: 12.8 μL

(4) A DNA ligation reaction kit (TaKaRa, 6022) was used to ligate the vector and the annealed product in the previous steps to obtain a sgRNA plasmid containing the gene target sequence.

(II) Gene Editing Process

1. Single-Cell Cloning Operating Steps for AAV Gene Knock-In

(1) Electrotransfection Procedure:

Donor cell preparation: Human pluripotent stem cells

Kit: Human Stem Cell Nucleofector® Kit 1

Instrument: Electrotransfection instrument

Medium: BioCISO

Inducible plasmid: s Cas9D10A, sgRNA clone AAVS1-1, sgRNA clone AAVS1-2, AAV S1 neo Vectol, and AAVS1 neo Vector II

Note: Inducible plasmids used for eGSH gene knock-in: Cas9D10A, sgRNA clone eGSH-1, sgRNA clone eGSH-2, and eGSH-neo/eGSH-puro(donor). Comparing the donor plasmid here with AAVS1, only the left and right recombination arms were different, and the other elements were the same. Since the gene editing process of eGSH was the same as that of AAVS1, it will not be repeated hereinbelow.

(2) The electrotransfected human pluripotent stem cells were screened in a dual-antibiotic medium containing G418 and puro.

(3) Single-cell clones were screened and cultured to obtain a single-cell clone strain.

2. Culture Reagent of AAV Gene Knock-In Single-Cell Clone Strain

(1) Medium: BioCISO+300 μg/ml G418+0.5 μg/ml puro

(It should be placed at room temperature in advance for 30-60 minutes in the dark until it returned to room temperature. Note: BioCISO should not be preheated at 37° C. to avoid reduced biomolecular activity.)

(2) Matrigel: hESC grade Matrigel

(Before cell passaging or resuscitation, a Matrigel working solution was added to a cell culture flask or culture dish and shaken until uniform, and it was ensured that the Matrigel completely covered the bottom of the culture flask or culture dish and that the Matrigel at anywhere could not be dried before use. In order to ensure that the cells could better adhere and survive, Matrigel should be placed in an incubator at 37° C. for a coating time, which was not less than 0.5 hours for 1:100× Matrigel and not be less than 2 hours for 1:200×Matrigel.)

(3) Digestion solution: EDTA was dissolved with DPBS to a final concentration of 0.5 mM, pH 7.4

(Note: EDTA could not be diluted with water; otherwise, cells would die due to reduced osmotic pressure.)

(4) Cryopreservative fluid: 60% BioCISO+30% ESCs grade FBS+10% DMSO

(It was preferred to prepare the cryopreservative fluid immediately before use.)

3. Process of Routine Maintenance Subculture

(1) Optimal Passage Time and Passage Ratio

a. Optimal time for passage: When the overall cell confluence reached 80% to 90%.

b. Optimal ratio for passage: 1:4 to 1:7 passage, and the optimal confluence on the next day should be maintained at 20% to 30%.

(2) Passaging Process

a. The Matrigel in the coated cell culture flask or culture dish was aspirated and discarded in advance, an appropriate amount of medium (BioCISO+300 μg/ml G418+0.5 μg/ml puro) was added, and the flask or dish was placed in an incubator at 37° C., 5% CO₂;

b. when the cells met passage requirements, the medium supernatant was aspirated, and an appropriate amount of 0.5 mM EDTA digestion solution was added to the cell flask or culture dish;

c. the cells were put into an incubator at 37° C., 5% CO₂ and incubated for 5-10 minutes (digested until most of the cells observed under a microscope were shrunk and rounded but not yet floating), and the cells were detached from the wall by gentle pipetting, then the cell suspension was aspirated into a centrifuge tube for centrifugation at 200 g for 5 min);

d. after centrifugation, the supernatant was discarded, the cells were resuspended with a medium, the cells were repeatedly gently pipetted several times until uniformly mixed, and the cells were then transferred to a Matrigel-coated flask or dish prepared in advance;

e. after the cells were transferred to the cell flask or culture dish, the flask or dish was shaken horizontally back and forth and from side to side, and after no abnormality was observed under the microscope, the cells were shaken uniform and placed in an incubator for culturing at 37° C., 5% CO₂; and

f. the next day, the adherence and survival state of the cells were observed, and the medium was aspirated and replaced every day on schedule as normal.

4. Cell Cryopreservation

(1) According to routine passaging operating steps, the cells were digested with 0.5 mM EDTA until most of the cells were shrunk and rounded but not yet floating, the cells were gently pipetted, the cell suspension was collected and centrifuged at 200 g for 5 minutes, the supernatant was discarded, an appropriate amount of cryopreservative fluid was added to resuspend the cells, and the cells were transferred to cryopreservation tubes (it was recommended to cryopreserve one tube for a 6-well plate confluence of 80%, and the volume of the cryopreservative fluid was 0.5 ml/tube);

(2) the cryopreservation tubes were placed in a programmed cooling box and immediately placed at −80° C. overnight (it was necessary to ensure that the temperature of the cryopreservation tubes decreased by 1° C. per minute); and

(3) the cells were immediately transferred into liquid nitrogen the next day.

5. Cell Resuscitation

(1) A Matrigel-coated cell flask or culture dish was prepared in advance; and before cell resuscitation, the Matrigel was aspirated, an appropriate amount of BioCISO was added to the cell flask or culture dish, and the cell flask or culture dish was incubated in an incubator at 37° C., 5% CO₂;

(2) the cryopreservation tubes were quickly taken out from the liquid nitrogen, immediately placed in a water bath at 37° C. and quickly shaken to quickly thaw the cells; and upon careful observation, when ice crystals disappeared completely, shaking was stopped, and the cells were transferred to a biological safety cabinet;

(3) 10 ml DMEM/F12 (1:1) basal medium was added to a 15 ml centrifuge tube in advance and equilibrated to room temperature, 1 ml DMEM/F12 (1:1) was pipetted by a Pasteur pipette, slowly added to the cryopreservation tube, and gently mixed, and the cell suspension was transferred to the prepared 15 ml centrifuge tube containing DMEM/F12 (1:1), and centrifuged at 200 g for 5 min;

(4) the supernatant was carefully discarded, an appropriate amount of BioCISO was added, the cells were gently mixed until uniform and seeded in the cell flask or culture dish prepared in advance, the flask or dish was shaken horizontally back and forth, and from side to side, and after no abnormality was observed under the microscope, the cells were shaken uniform and placed in an incubator for culturing at 37° C., 5% CO₂; and

(5) the next day, the adherence and survival state of the cells were observed, and the medium was replaced every day on schedule as normal. If the adherence was good, BioCISO was replaced with BioCISO+300 μg/ml G418+0.5 μg/ml puro.

(III) Detection Method of AAVS1 Gene Knock-In

1. Detection of Single-Cell Clone AAVS1 Gene Knock-In

(1) Detection Instructions for AAVS1 Gene Knock-In

a. Test objective: gene-knock-in-treated cells were detected by PCR to test whether the cells were homozygous. Since the two donor fragments only had differences in terms of resistance gene sequence, in order to determine whether the cells were homozygous (the two chromosomes were respectively knocked in with donor fragments of different resistance genes), it was necessary to detect whether the genome of the cells contained the donor fragments of the two resistance genes, and only cells with dual knock-in were likely to be correct homozygotes.

b. Test method: First of all, a primer was designed inside the donor plasmid (non-recombination-arm part), and another primer was then designed in the genome PPP1R12C (non-recombination-arm part). If the donor fragments could be inserted correctly in the genome, there would be bands of interest; otherwise, no bands of interest would appear).

c. The primer sequences and PCR protocols in the test scheme were shown in Table 8:

TABLE 8
Primer sequences and PCR protocols in the test scheme

| | | | | | PCR |
|---|---|---|---|---|---|
| | Primer | | Sequence | Product | conditions |
| No. | abbreviation | Sequence (5′ 3′) | number | (bp) | (Phanta enzyme) |
| 1 | F1 | CCATAGCTCAGTCTGG | SEQ ID NO. 99 | 1578 | Annealing at |
| | | TCTATC | | | 58° C., |
| 2 | R1 | TCAGGATGATCTGGAC | SEQ ID NO. 100 | | extension for |
| | | GAAGAG | | | 1 min, and |
| | | | | | 30 cycles |
| 3 | F2 | CCGGTCCTGGACTTTG | SEQ ID NO. 101 | 1728 | Annealing at |
| | | TCTC | | | 62° C., |
| 4 | R2 | CTCGACATCGGCAAGG | SEQ ID NO. 102 | | extension for |
| | | TGTG | | | 1 min 30 sec, |
| | | | | | and 30 cycles |
| 5 | F2 | CGCATTGGAGTCGCTT | SEQ ID NO. 103 | 1874 | Annealing at |
| | (Outer) | TAAC | | | 60° C., |
| 6 | R2 | CGAGCTGCAAGAACTC | SEQ ID NO. 104 | | extension for |
| | (Outer) | TTCCTCAC | | | 1 min 30 sec, |
| | | | | | and 18 cycles |
| 7 | F3 | CACGGCACTTACCTGT | SEQ ID NO. 105 | 2151 | Annealing at |
| | (Outer) | GTTCTGG | | | 60° C., |
| 8 | R3 | CAGTACAGGCATCCCT | SEQ ID NO. 106 | | extension for |
| | (Outer) | GTGAAAG | | | 1 min 30 sec, |
| | | | | | and 18 cycles |

Note:
The detection method for eGSH gene knock-in was the same as the detection
principle and method for AAVS1 gene knock-in, and will not be described here.

VIII. Test Method for Gene Knock-In at Genomic Safe Locus

(1) Test objective: gene-knock-in-treated cells were detected by PCR to test whether the cells were homozygous. Since the two donor fragments only had differences in terms of resistance gene sequence, in order to determine whether the cells were homozygous (the two chromosomes were respectively knocked in with donor fragments of different resistance genes), it was necessary to detect whether the genome of the cells contained the donor fragments of the two resistance genes, and only cells with dual knock-in were likely to be correct homozygotes.

(2) Test method: First, a primer was designed inside the donor plasmid (non-recombination-arm part), and another primer was then designed in the genome (non-recombination-arm part). If the donor fragments could be inserted correctly in the genome, there would be bands of interest; otherwise, no bands of interest would appear.

IX. Test Method for Allogeneic Immunological Compatibility Effect (Specific Immunity and Innate Immunity)

1. Preparation of Effector Cells

Blood was drawn from volunteers to isolate T cells and NK cells. Effector cells and immunologically compatible pluripotent stem cells were derived from different people.

1) T cell isolation: Human peripheral blood mononuclear cells (PBMC) were isolated using Ficoll-hypaque density gradient centrifugation, and T cells were isolated using Dynabeads™ CD3 kit (Invitrogen™, Cat. No. 11151D). The cells were resuspended in RPMI1640 medium containing 10% FBS, and the cells were counted by trypan blue staining and concentrated to 1×10⁷ cells/mL.

2) NK cell isolation: NK cells were sorted and isolated using MagniSort™ Human NK cell Enrichment Kit (Invitrogen™, Cat. No. 8804-6819-74). The cells were resuspended in RPMI1640 medium containing 10% FBS, and the cells were counted by trypan blue staining and concentrated to 1×10⁷ cells/mL.

2. Preparation of Target Cells

Embryoid body cells prepared from PSCs were taken, digested and resuspended, and the cells were counted by trypan blue staining and prepared into a cell suspension of 1×10⁷ cells/mL.

3. The ⁵¹Cr Release Assay

When normal cells came into contact with T/NK cells (allogeneic), T/NK attacked the normal cells and caused cell lysis and death. However, if there was good immunological compatibility, no attack by T/NK would occur, that is, immune escape. Therefore, the detection of the amount of ⁵¹Cr in the medium could reflect immunological compatibility. The less the amount of ⁵¹Cr released into the detection medium, the better the immunological compatibility.

Cell-mediated cytotoxicity was quantitatively detected, wherein target cells were labeled with the radioisotope ⁵¹Cr and co-incubated with effector molecules or cells, and the cytotoxic activity was determined according to the radiation pulse count (cpm) of ⁵¹Cr released by target cell lysis.

1) The target cells were labeled with 100 μCi (Ci, unit of radioactivity) Na⁵¹CrO₄ at 37° C. for 120 min and shaken every 15 min; after labeling, centrifugation was further carried out 5 times with a washing solution; and finally, the cells were resuspended in a culture solution to 1×10⁶ cells/mL for use.

2) The target cells and T/NK cells were added to a 96-well culture plate, wherein 100 μl of target cells (2.5×10³ cells) and 100 μl of effector cells (E/T=1:2, 1:5, or 1:10, E/T was the ratio of target cells to effector cells (T/NK)), and a natural release control well (100 μl target cells+100 μl medium) and a maximum release well (100 μl target cells+100 μl 2% SDS) were established. They were placed at 37° C., 5% CO₂ for 4 h for incubation. After they were taken out, the supernatant was aspirated from each well with a pipette and centrifuged, 100 μl of supernatant was taken to measure the cpm value with a γ counter.

Note: Generally, the natural release rate of ⁵¹Cr was required to be less than 10%.

3) Calculation of results: The natural release rate of ⁵¹Cr and the activity of T/NK cells were calculated according to the formulas:

$${}^{51} Cr natural release rate ( \% ) = \frac{cpm value of natural release control well}{cpm value of maximal release control well} \times 100 \% \\ Activity of T / NK cell ( \% ) = \frac{cpm value of experimental well - cpm value of natural release control well}{cpm value of maximal release control well - cpm value of natural release control well} \times 100 \%$$

4. The CFSE Test Assay

The fluorescent dye CFSE, also known as CFDA SE (5,6-carboxyfluorescein diacetate, succinimidyl ester, hydroxyfluorescein diacetate succinimidyl ester), was a fluorescent dye that could penetrate cell membranes and could be detected by flow cytometry.

1) A CFSE working solution (with a final concentration of 5 μmol/L CFSE) was added to the target cells, and the cells were incubated at 37° C., 5% CO₂ for 10 min, and washed twice. After trypan blue staining and counting, the cells were resuspended in a medium and prepared into 1×10⁶ cells/mL for later use.

2) The target cells and the effector cells were added to 5 ml flow tubes, wherein 100 μl of target cells (1×10⁵ ml⁻¹) and effector cells (E/T=1:2, 1:5, or 1:10, E/T is the ratio of target cells to effector cells (T)) were added to each tube, and the flow tube in which only target cells were added was used as a control. The effector cells and the target cells in the flow tubes were gently mixed until uniform. PI was added, and they were placed at 37° C., 5% CO₂ for 4 h for incubation. The percentage of CFSE⁺PI⁺ cells (dead target cells) was detected by flow cytometry.

Target cell death rate (%)=death rate of target cells stimulated with T cells (%)−the natural death rate of target cells (%)

5. Analysis of CD₁₀₇ₐ Expression in NK Cells by Flow Cytometer (FCM)

When the NK cells killed the target cells, CD₁₀₇ₐ molecules were transported to the surface of the cell membrane, and NK cells with positive CD₁₀₇ₐ molecule expression could represent NK cells with killing activity.

1) The effector cells and the target cells were mixed at a certain ratio (E/NK=3:1, 1:1, or 1:3, E/NK was the ratio of target cells to effector cells (NK)), placed in culture wells, and incubated at 37° C., 5% CO₂ for 2 h, monensin (2 μmol/L) was added for continued incubation for 3.5 h, and PE-Cy5-CD₁₀₇ₐ and FITC-CD56 antibodies were then added for incubation for 30 min. After washing three times with PBS buffer, the cells were fixed with 200 μL of 1% paraformaldehyde for flow analysis. As a positive control, the effector cells were simultaneously stimulated with only PMA (2.5 μg/mL) and ionomycin (0.5 μg/mL).

Note: The natural expression frequency of CD₁₀₇ₐ on the surface of the NK cell membrane was very low, about 1.2% to 5.8%.

2) Calculation of Results

NK cell cytotoxicity=CD₁₀₇ₐ positive rate upon stimulation by target cells (%)−CD₁₀₇ₐ natural expression rate (%)

X. MTT Experiment for Detecting Cell Viability

After digestion and cell counting, the cells were blown uniform with a corresponding medium and plated into a 96-well plate in which each well was seeded with 3000 cells, with 5 duplicate wells, the wells were then supplemented with the corresponding medium to a final volume of 150 uL, the corresponding medium was changed every day, the cells were placed in an incubator at 37° C., 5% CO₂ for 72 h, then the MTT value was measured.

### Example 2

The Tet-Off system and a combination of immunologically compatible molecules (Tables 3-6) were knocked into the genomic safe locus AAVS1 of hPSC-derived EBs by means of the method of Example 1 to obtain EB sphere immunologically compatible cells.

⁵¹Cr release assay was used to detect the effect of immunological compatibility between the EB sphere immunologically compatible cells and T cells:

1. The EB sphere immunologically compatible cells were digested into individual cells as target cells;

2. ⁵¹Cr-labeled target cells and T cells were added to a 96-well culture plate at a ratio of 1:5 for post-reaction detection; and

3. the cell-specific ⁵¹Cr release rate of the EB sphere immunologically compatible cells was detected according to the ⁵¹Cr release assay, and the results are shown in Table 9 and FIG. 8.

TABLE 9
⁵¹Cr release rate of EB sphere immunologically
compatible cells

| | | | Independent |
|---|---|---|---|
| | Mean ⁵¹Cr | | sample |
| | release rate | Deviation | t-test |
| Group | (%) | (±) | (*p < 0.01) |
| N | 60.23 | 1.82 | |
| (control) | | | |
| Aa1 | 44.80 | 1.10 | * |
| Aa2 | 43.15 | 0.41 | * |
| Ab1 | 45.20 | 1.44 | * |
| Ab2 | 41.89 | 0.66 | * |
| Ac1 | 42.32 | 1.24 | * |
| Ac2 | 40.14 | 1.44 | * |
| Ad1 | 45.75 | 0.33 | * |
| Ad2 | 44.63 | 0.30 | * |
| Ae1 | 45.74 | 0.34 | * |
| Ae2 | 44.79 | 1.06 | * |
| Af1 | 44.81 | 0.06 | * |
| Af2 | 43.42 | 0.38 | * |
| Ag1 | 42.68 | 0.09 | * |
| Ag2 | 42.74 | 0.03 | * |
| Ah | 41.69 | 0.05 | * |
| Ai1 | 38.71 | 0.27 | * |
| Ai2 | 40.47 | 0.86 | * |
| Ai3 | 37.49 | 0.82 | * |
| Ai4 | 38.85 | 0.78 | * |
| Ai5 | 39.73 | 1.04 | * |
| Ai6 | 37.43 | 0.02 | * |
| B1 | 35.00 | 1.30 | * |
| B2 | 34.47 | 0.33 | * |
| B3 | 35.22 | 0.23 | * |
| B4 | 35.29 | 0.04 | * |
| B5 | 35.49 | 0.22 | * |
| C1 | 30.09 | 0.19 | * |
| C2 | 33.58 | 0.38 | * |
| C3 | 32.22 | 0.06 | * |
| C4 | 30.54 | 0.37 | * |
| C5 | 30.22 | 0.05 | * |
| C6 | 31.12 | 0.11 | * |
| C7 | 31.18 | 1.68 | * |
| D1 | 29.10 | 0.94 | * |
| D2 | 32.28 | 1.21 | * |
| D3 | 31.18 | 0.12 | * |

It can be seen in Table 9 and FIG. 8 that the immunological compatibility effect of the hPSC-derived EB sphere cells that we prepared was significant, and especially, Groups C1, C4, C5 and D1 were the most significant.

### Example 3

In this example, where a transplant became diseased, the effect of non-expression of the knocked-in immunologically compatible molecule after adding an inducer (Dox) was tested. The steps were as follows:

(1) 6 μM of Dox was added to a medium of the EB sphere immunologically compatible cells prepared in Example 2 for 48 h of treatment;

(2) the Dox-treated EB sphere immunologically compatible cells were digested into individual cells as target cells;

(3)⁵¹Cr-labeled target cells and T cells were added to a 96-well culture plate at a ratio of 1:5 for post-reaction detection; and

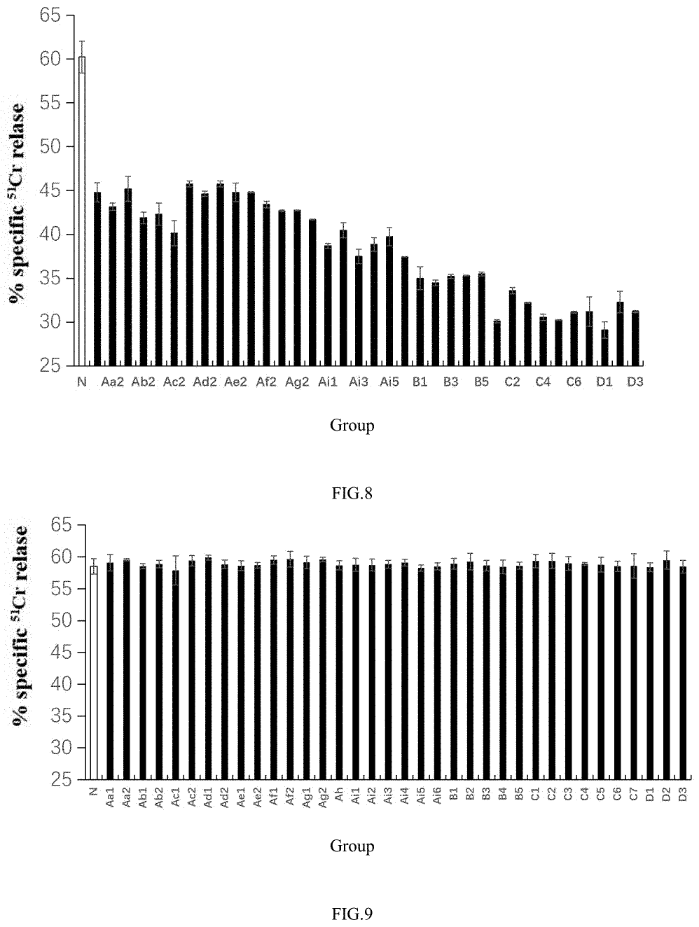

(4) the cell-specific ⁵¹Cr release rate of the EB sphere immunologically compatible cells was detected according to the ⁵¹Cr release assay, and the results are shown in Table 10 and FIG. 9.

TABLE 10
⁵¹Cr release rate of Dox-treated EB sphere
immunologically compatible cells

| | Mean ⁵¹Cr | Deviation |
|---|---|---|
| Group | release rate (%) | (±) |
| N | 58.50 | 1.22 |
| (control) | | |
| Aa1 | 59.10 | 1.29 |
| Aa2 | 59.56 | 0.16 |
| Ab1 | 58.56 | 0.42 |
| Ab2 | 58.89 | 0.57 |
| Ac1 | 57.88 | 2.28 |
| Ac2 | 59.41 | 0.84 |
| Ad1 | 59.92 | 0.39 |
| Ad2 | 58.83 | 0.66 |
| Ae1 | 58.62 | 0.78 |
| Ae2 | 58.70 | 0.45 |
| Af1 | 59.52 | 0.65 |
| Af2 | 59.63 | 1.24 |
| Ag1 | 59.13 | 0.98 |
| Ag2 | 59.59 | 0.37 |
| Ah | 58.67 | 0.71 |
| Ai1 | 58.77 | 1.03 |
| Ai2 | 58.73 | 0.92 |
| Ai3 | 58.86 | 0.60 |
| Ai4 | 59.11 | 0.52 |
| Ai5 | 58.24 | 0.49 |
| Ai6 | 58.51 | 0.53 |
| B1 | 58.94 | 0.85 |
| B2 | 59.26 | 1.31 |
| B3 | 58.65 | 0.83 |
| B4 | 58.44 | 1.07 |
| B5 | 58.63 | 0.54 |
| C1 | 59.35 | 1.04 |
| C2 | 59.36 | 1.18 |
| C3 | 58.99 | 1.07 |
| C4 | 58.93 | 0.23 |
| C5 | 58.79 | 1.17 |
| C6 | 58.53 | 0.82 |
| C7 | 58.58 | 1.94 |
| D1 | 58.41 | 0.69 |
| D2 | 59.51 | 1.46 |
| D3 | 58.48 | 0.99 |

It can be seen in Table 10 and FIG. 9 that after the hPSC-derived EB sphere immunologically compatible cells that we prepared were treated with the inducer (Dox), the immunologically compatible molecule sequence knocked therein exhibited non-expression effects. Thus, reversible regulation of immunological compatibility in cells was achieved.

### Example 4

The Tet-Off system and a combination of immunologically compatible molecules (Tables 3-6) were knocked into the genomic safe locus AAVS1 of hPSC-derived EBs by means of the method of Example 1 to obtain EB sphere immunologically compatible cells.

⁵¹Cr release assay was used to detect the effect of immunological compatibility between the EB sphere immunologically compatible cells and NK cells:

1. The EB sphere immunologically compatible cells were digested into individual cells as target cells;

2. ⁵¹Cr-labeled target cells and T cells were added to a 96-well culture plate at a ratio of 1:5 for post-reaction detection; and

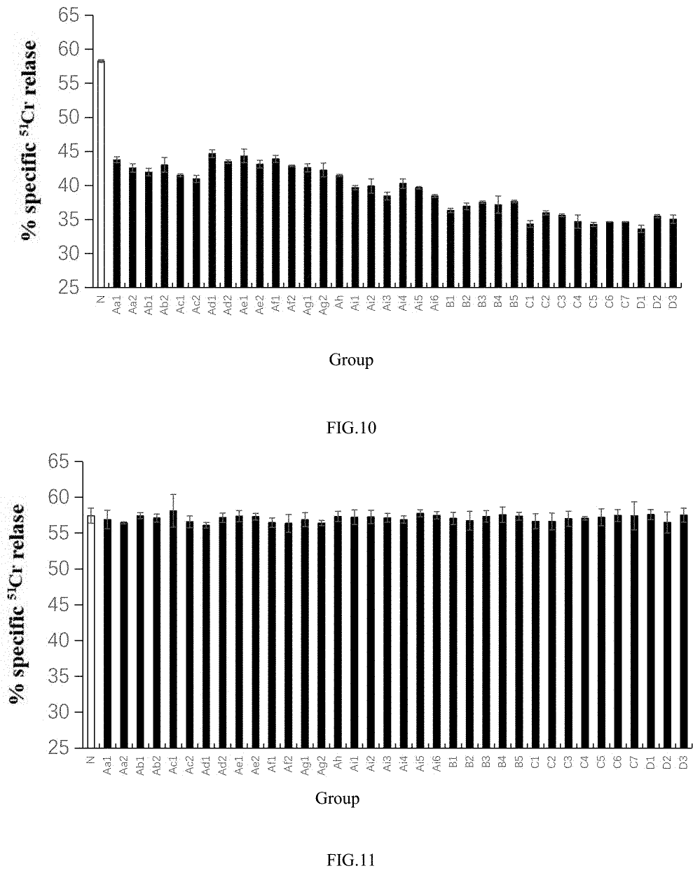

3. the cell-specific ⁵¹Cr release rate of the EB sphere immunologically compatible cells was detected according to the ⁵¹Cr release assay, and the results are shown in Table 11 and FIG. 10.

TABLE 11
⁵¹Cr release rate of EB sphere immunologically
compatible cells

| | | | Independent |
|---|---|---|---|
| | Mean ⁵¹Cr | | sample |
| | release rate | Deviation | t-test |
| Group | (%) | (±) | (*p < 0.01) |
| N | 58.26 | 0.22 | |
| (control) | | | |
| Aa1 | 43.76 | 0.45 | * |
| Aa2 | 42.56 | 0.60 | * |
| Ab1 | 41.97 | 0.56 | * |
| Ab2 | 43.01 | 1.09 | * |
| Ac1 | 41.48 | 0.21 | * |
| Ac2 | 40.97 | 0.53 | * |
| Ad1 | 44.66 | 0.57 | * |
| Ad2 | 43.50 | 0.26 | * |
| Ae1 | 44.34 | 1.00 | * |
| Ae2 | 43.12 | 0.57 | * |
| Af1 | 43.91 | 0.51 | * |
| Af2 | 42.88 | 0.11 | * |
| Ag1 | 42.59 | 0.57 | * |
| Ag2 | 42.26 | 1.00 | * |
| Ah | 41.45 | 0.18 | * |
| Ai1 | 39.69 | 0.32 | * |
| Ai2 | 39.92 | 1.07 | * |
| Ai3 | 38.46 | 0.58 | * |
| Ai4 | 40.28 | 0.70 | * |
| Ai5 | 39.67 | 0.17 | * |
| Ai6 | 38.47 | 0.20 | * |
| B1 | 36.35 | 0.30 | * |
| B2 | 36.94 | 0.49 | * |
| B3 | 37.54 | 0.18 | * |
| B4 | 37.18 | 1.27 | * |
| B5 | 37.64 | 0.17 | * |
| C1 | 34.35 | 0.48 | * |
| C2 | 35.98 | 0.32 | * |
| C3 | 35.62 | 0.23 | * |
| C4 | 34.69 | 0.97 | * |
| C5 | 34.27 | 0.31 | * |
| C6 | 34.66 | 0.02 | * |
| C7 | 34.65 | 0.03 | * |
| D1 | 33.63 | 0.55 | * |
| D2 | 35.49 | 0.23 | * |
| D3 | 35.05 | 0.61 | * |

It can be seen in Table 11 and FIG. 10 that the immunological compatibility effect of the EB sphere cells that we prepared was significant, and especially, Groups C1, C4, C5 and D1 were the most significant.

### Example 5

In this example, when a transplant became diseased, the effect of non-expression of the knocked-in immunologically compatible molecule was tested after adding an inducer (Dox). The steps were as follows:

(1) 6 μM of Dox was added to a medium of the EB sphere immunologically compatible cells prepared in Example 4 for 48 h of treatment;

(2) the Dox-treated EB sphere immunologically compatible cells were digested into individual cells as target cells;

(3) ⁵¹Cr-labeled target cells and NK cells were added to a 96-well culture plate at a ratio of 1:5 for post-reaction detection; and

(4) the cell-specific ⁵¹Cr release rate of the EB sphere immunologically compatible cells was detected according to the ⁵¹Cr release assay, and the results are shown in Table 12 and FIG. 11.

TABLE 12
⁵¹Cr release rate of Dox-treated EB sphere immunologically
compatible cells

| | Mean ⁵¹Cr | |
|---|---|---|
| | release rate | Deviation |
| Group | (%) | (±) |
| N (control) | 57.44 | 1.03 |
| Aa1 | 56.90 | 1.29 |
| Aa2 | 56.44 | 0.16 |
| Ab1 | 57.44 | 0.42 |
| Ab2 | 57.11 | 0.57 |
| Ac1 | 58.12 | 2.28 |
| Ac2 | 56.59 | 0.84 |
| Ad1 | 56.08 | 0.39 |
| Ad2 | 57.17 | 0.66 |
| Ae1 | 57.38 | 0.78 |
| Ae2 | 57.30 | 0.45 |
| Af1 | 56.48 | 0.65 |
| Af2 | 56.37 | 1.24 |
| Ag1 | 56.87 | 0.98 |
| Ag2 | 56.41 | 0.37 |
| Ah | 57.33 | 0.71 |
| Ai1 | 57.23 | 1.03 |
| Ai2 | 57.27 | 0.92 |
| Ai3 | 57.14 | 0.60 |
| Ai4 | 56.89 | 0.52 |
| Ai5 | 57.76 | 0.49 |
| Ai6 | 57.49 | 0.53 |
| B1 | 57.06 | 0.85 |
| B2 | 56.74 | 1.31 |
| B3 | 57.35 | 0.83 |
| B4 | 57.56 | 1.07 |
| B5 | 57.37 | 0.54 |
| C1 | 56.65 | 1.04 |
| C2 | 56.64 | 1.18 |
| C3 | 57.01 | 1.07 |
| C4 | 57.07 | 0.23 |
| C5 | 57.21 | 1.17 |
| C6 | 57.47 | 0.82 |
| C7 | 57.42 | 1.94 |
| D1 | 57.59 | 0.69 |
| D2 | 56.49 | 1.46 |
| D3 | 57.52 | 0.99 |

It can be seen in Table 12 and FIG. 11 that when the EB sphere immunologically compatible cells that we prepared were treated with the inducer (Dox), the immunologically compatible molecule sequence knocked therein exhibited non-expression effects. Thus, reversible regulation of immunological compatibility in cells was achieved.

### Example 6

In this example, the CFSE test assay was used to detect the immunological compatibility effect of EB sphere immunologically compatible cells:

1. The EB sphere immunologically compatible cells prepared in Example 2 were digested into individual cells as target cells;

2. CFSE-labeled target cells and T cells were added to a 5 ml flow tube at a ratio of 1:5 for reaction and then detection; and

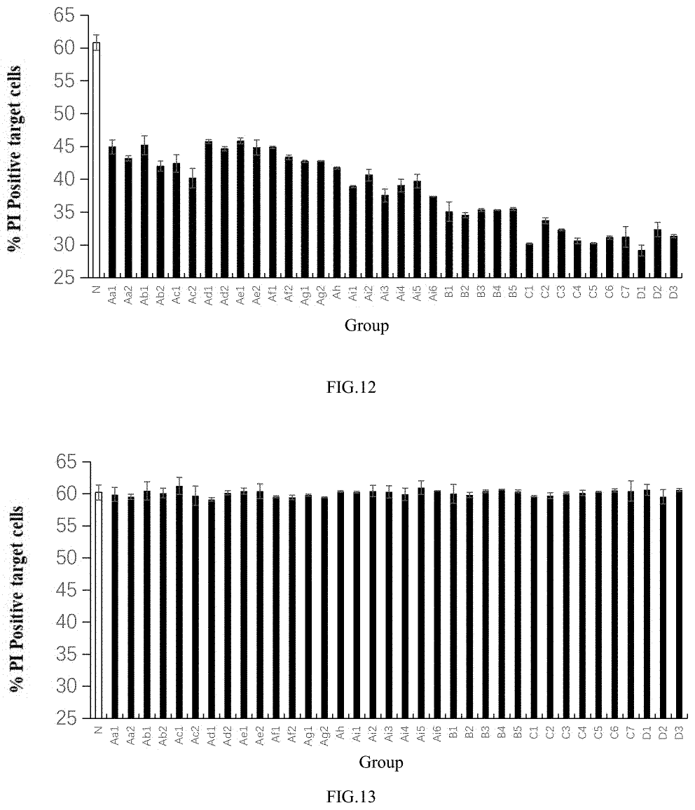

3. the percentage of CFSE⁺PI⁺ cells (dead target cells) in the EB sphere immunologically compatible cells was detected according to the CFSE test assay, and the results are shown in Table 13 and FIG. 12.

TABLE 13
CFSE test results of EB sphere immunologically
compatible cells

| | | | Independent |
|---|---|---|---|
| | Mean death | | sample |
| | rate of target | Deviation | t-test |
| Group | cell (%) | (±) | (*p < 0.01) |
| N | 60.81 | 1.17 | |
| (control) | | | |
| Aa1 | 44.91 | 1.08 | * |
| Aa2 | 43.17 | 0.40 | * |
| Ab1 | 45.20 | 1.44 | * |
| Ab2 | 42.01 | 0.77 | * |
| Ac1 | 42.42 | 1.34 | * |
| Ac2 | 40.19 | 1.50 | * |
| Ad1 | 45.74 | 0.28 | * |
| Ad2 | 44.65 | 0.33 | * |
| Ae1 | 45.84 | 0.45 | * |
| Ae2 | 44.84 | 1.16 | * |
| Af1 | 44.87 | 0.15 | * |
| Af2 | 43.36 | 0.36 | * |
| Ag1 | 42.72 | 0.16 | * |
| Ag2 | 42.78 | 0.06 | * |
| Ah | 41.73 | 0.14 | * |
| Ai1 | 38.88 | 0.16 | * |
| Ai2 | 40.64 | 0.87 | * |
| Ai3 | 37.54 | 0.97 | * |
| Ai4 | 39.07 | 0.96 | * |
| Ai5 | 39.71 | 1.06 | * |
| Ai6 | 37.40 | 0.03 | * |
| B1 | 35.07 | 1.48 | * |
| B2 | 34.53 | 0.40 | * |
| B3 | 35.33 | 0.22 | * |
| B4 | 35.31 | 0.07 | * |
| B5 | 35.48 | 0.23 | * |
| C1 | 30.18 | 0.11 | * |
| C2 | 33.70 | 0.46 | * |
| C3 | 32.31 | 0.15 | * |
| C4 | 30.63 | 0.41 | * |
| C5 | 30.27 | 0.08 | * |
| C6 | 31.13 | 0.24 | * |
| C7 | 31.23 | 1.58 | * |
| D1 | 29.16 | 0.85 | * |
| D2 | 32.35 | 1.11 | * |
| D3 | 31.33 | 0.24 | * |

It can be seen in Table 13 and FIG. 12 that the immunological compatibility effect of the EB sphere cells that we prepared was significant, and especially, Groups C1, C4, C5 and D1 were the most significant.

### Example 7

In this example, when a transplant became diseased, the effect of non-expression of the knocked-in immunologically compatible molecule after using an inducer (Dox) was tested in the CFSE test assay. The steps were as follows:

(1) 6 μM of Dox was added to a medium of the EB sphere immunologically compatible cells prepared in Example 2 for 48 h of treatment;

(2) the Dox-treated EB sphere immunologically compatible cells were digested into individual cells as target cells;

(3) CFSE-labeled target cells and T cells were added to a 5 ml flow tube at a ratio of 1:5 for reaction and then detection; and

(4) the percentage of CFSE⁺PI⁺ cells (dead target cells) in the hPSC-derived EB sphere cells was detected according to the CFSE test assay, and the results are shown in Table 14 and FIG. 13.

TABLE 14
CFSE test results of Dox-treated EB sphere immunologically
compatible cells

| | Mean death | |
|---|---|---|
| | rate of target | Deviation |
| Group | cell (%) | (±) |
| N (control) | 60.21 | 1.08 |
| Aa1 | 59.90 | 1.35 |
| Aa2 | 59.56 | 0.23 |
| Ab1 | 60.45 | 0.41 |
| Ab2 | 60.11 | 0.54 |
| Ac1 | 61.23 | 2.39 |
| Ac2 | 59.69 | 0.75 |
| Ad1 | 59.13 | 0.46 |
| Ad2 | 60.16 | 0.71 |
| Ae1 | 60.41 | 0.72 |
| Ae2 | 60.40 | 0.51 |
| Af1 | 59.53 | 0.66 |
| Af2 | 59.43 | 1.15 |
| Ag1 | 59.81 | 1.00 |
| Ag2 | 59.45 | 0.49 |
| Ah | 60.37 | 0.66 |
| Ai1 | 60.26 | 0.94 |
| Ai2 | 60.44 | 1.02 |
| Ai3 | 60.31 | 0.58 |
| Ai4 | 59.94 | 0.43 |
| Ai5 | 60.98 | 0.86 |
| Ai6 | 60.46 | 0.55 |
| B1 | 60.03 | 0.85 |
| B2 | 59.81 | 1.46 |
| B3 | 60.40 | 0.76 |
| B4 | 60.67 | 1.06 |
| B5 | 60.39 | 0.63 |
| C1 | 59.63 | 1.10 |
| C2 | 59.73 | 1.30 |
| C3 | 60.12 | 1.16 |
| C4 | 60.16 | 0.33 |
| C5 | 60.30 | 1.17 |
| C6 | 60.53 | 0.83 |
| C7 | 60.43 | 1.97 |
| D1 | 60.64 | 0.62 |
| D2 | 59.55 | 1.46 |
| D3 | 60.58 | 0.84 |

It can be seen in Table 14 and FIG. 13 that when the hPSC-derived EB sphere immunologically compatible cells that we prepared were treated with the inducer (Dox), the immunologically compatible molecule sequence knocked therein exhibited non-expression effects.

### Example 8

In this example, the expression of CD₁₀₇ₐ in NK cells was analyzed using the flow cytometer (FCM) to detect the immunological compatibility effect of the EB sphere immunologically compatible cells prepared in Example 2:

1. The EB sphere immunologically compatible cells were digested into individual cells as target cells;

2. the target cells and NK cells were added to a 5 ml flow tube at a ratio of 1:1 for reaction and then detection; and

3. the cytotoxicity of the NK cells was detected according to the expression of CD₁₀₇ₐ in the NK cells, and the results are shown in Table 15 and FIG. 14.

TABLE 15
Detection results of CD₁₀₇ₐ expression in
NK cells

| | Mean | | Independent |
|---|---|---|---|
| | cytotoxicity | | sample |
| | of NK cell | Deviation | t-test |
| Group | (%) | (±) | (*p < 0.01) |
| N | 55.81 | 1.17 | |
| (control) | | | |
| Aa1 | 39.22 | 0.97 | * |
| Aa2 | 36.70 | 0.40 | * |
| Ab1 | 38.93 | 1.23 | * |
| Ab2 | 36.39 | 0.77 | * |
| Ac1 | 35.91 | 1.16 | * |
| Ac2 | 35.88 | 1.50 | * |
| Ad1 | 38.87 | 0.28 | * |
| Ad2 | 37.92 | 0.33 | * |
| Ae1 | 38.97 | 0.45 | * |
| Ae2 | 38.45 | 1.13 | * |
| Af1 | 38.30 | 0.15 | * |
| Af2 | 36.93 | 0.36 | * |
| Ag1 | 36.07 | 0.16 | * |
| Ag2 | 36.31 | 0.06 | * |
| Ah | 35.22 | 0.12 | * |
| Ai1 | 32.65 | 0.16 | * |
| Ai2 | 32.62 | 0.86 | * |
| Ai3 | 29.66 | 0.96 | * |
| Ai4 | 31.97 | 0.96 | * |
| Ai5 | 33.50 | 1.06 | * |
| Ai6 | 31.45 | 0.17 | * |
| B1 | 29.53 | 1.48 | * |
| B2 | 28.90 | 0.40 | * |
| B3 | 29.62 | 0.22 | * |
| B4 | 29.66 | 0.07 | * |
| B5 | 29.94 | 0.22 | * |
| C1 | 24.80 | 0.11 | * |
| C2 | 28.26 | 0.29 | * |
| C3 | 27.25 | 0.52 | * |
| C4 | 25.01 | 0.41 | * |
| C5 | 25.29 | 0.08 | * |
| C6 | 26.33 | 0.24 | * |
| C7 | 25.14 | 1.58 | * |
| D1 | 24.21 | 0.85 | * |
| D2 | 27.06 | 1.11 | * |
| D3 | 26.19 | 0.24 | * |

It can be seen in Table 15 and FIG. 14 that the immunological compatibility effect of the hPSC-derived EB sphere cells that we prepared was significant, and especially, Groups C1, C4, C5 and D1 were the most significant.

### Example 9

In this example, when a transplant became diseased, the effect of non-expression of the knocked-in immunologically compatible molecule after using an inducer (Dox) was tested by the analysis of CD₁₀₇ₐ expression in NK cells using the flow cytometer (FCM). The steps were as follows:

(1) 6 μM of Dox was added to a medium of the EB sphere immunologically compatible cells prepared in Example 2 for 48 h of treatment;

(2) the Dox-treated EB sphere immunologically compatible cells were digested into individual cells as target cells;

(3) the target cells and NK cells were added to a 5 ml flow tube at a ratio of 1:1 for reaction and then detection; and

(4) the cytotoxicity of the NK cells was detected according to the expression of CD₁₀₇ₐ in the NK cells, and the results are shown in Table 16 and FIG. 15.

TABLE 16
Detection results of CD₁₀₇ₐ expression in Dox-treated NK cells

| | Mean | |
|---|---|---|
| | cytotoxicity of | Deviation |
| Group | NK cell (%) | (±) |
| N (control) | 55.43 | 1.08 |
| Aa1 | 54.68 | 1.46 |
| Aa2 | 54.35 | 0.47 |
| Ab1 | 55.05 | 1.06 |
| Ab2 | 55.32 | 0.21 |
| Ac1 | 56.70 | 2.02 |
| Ac2 | 54.68 | 0.75 |
| Ad1 | 54.99 | 1.94 |
| Ad2 | 55.45 | 0.24 |
| Ae1 | 55.54 | 0.61 |
| Ae2 | 55.76 | 0.13 |
| Af1 | 54.61 | 0.72 |
| Af2 | 54.28 | 1.02 |
| Ag1 | 54.32 | 0.22 |
| Ag2 | 54.62 | 0.62 |
| Ah | 55.18 | 0.85 |
| Ai1 | 54.69 | 0.21 |
| Ai2 | 55.55 | 0.85 |
| Ai3 | 55.10 | 0.70 |
| Ai4 | 54.59 | 0.23 |
| Ai5 | 55.91 | 0.97 |
| Ai6 | 55.07 | 0.37 |
| B1 | 55.20 | 0.64 |
| B2 | 55.47 | 0.40 |
| B3 | 55.29 | 0.57 |
| B4 | 54.94 | 0.57 |
| B5 | 55.53 | 0.40 |
| C1 | 55.40 | 1.25 |
| C2 | 54.89 | 1.17 |
| C3 | 55.59 | 0.47 |
| C4 | 55.26 | 0.35 |
| C5 | 55.01 | 1.01 |
| C6 | 55.99 | 0.28 |
| C7 | 54.43 | 1.21 |
| D1 | 54.96 | 1.63 |
| D2 | 55.55 | 0.71 |
| D3 | 55.87 | 1.11 |

It can be seen in Table 16 and FIG. 15 that when the hPSC-derived EB sphere immunologically compatible cells that we prepared were treated with the inducer (Dox), the immunologically compatible molecule sequence knocked therein exhibited non-expression effects.

### Example 10

Through Examples 2-9, it could be basically determined that the immunological compatibility effects of Groups C1, C4, C5 and D1 were the best. Comprehensively considering factors such as the difficulty of constructing immunologically compatible cells, the scheme of Group D1 was selected as an example hereinafter to test the effect of immunological compatibility between the allogeneic hPSCs and hPSC-derived derivatives (hPSCs-MSCs, NSCs and EBs) (treated by the technical solution of the present disclosure) and T cells.

By means of the technical solution of Example 1, the immunologically compatible molecule of Group D1 was knocked into the genomic safe locus AAVS1 of hPSCs, hPSCs-MSCs, NSCs, and EB cells, and the cells were digested into individual cells as target cells.

⁵¹Cr release assay was used to detect the immunological compatibility effect of the target cells:

1. ⁵¹Cr-labeled target cells and T cells were added to a 96-well culture plate at a ratio of 1:5 for post-reaction detection; and

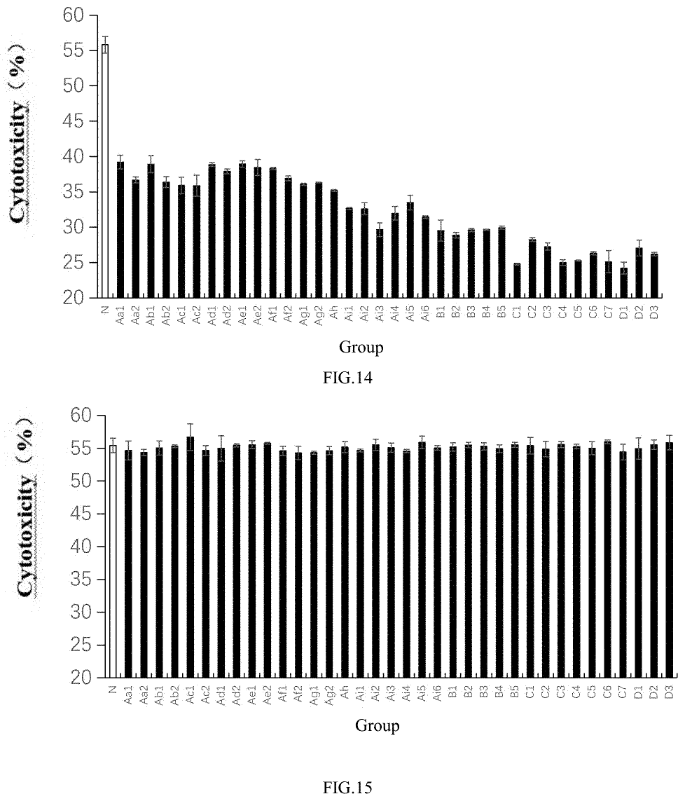

2. the cell-specific ⁵¹Cr release rate of the target cells was detected according to the ⁵¹Cr release assay, and the results are shown in Table 17 and FIG. 16.

TABLE 17
⁵¹Cr release rates of hPSC-MSC, NSC and EB immunologically
compatible cells

| | | Mean ⁵¹Cr | |
|---|---|---|---|
| | Group | release rate (%) | Deviation (±) |
| hPSCs | 1 | 52.49 | 1.55 |
| | 2 | 29.60 | 0.57 |
| | 3 | 52.98 | 0.69 |
| hPSCs-MSCs | 1 | 50.04 | 0.73 |
| | 2 | 27.86 | 0.69 |
| | 3 | 48.89 | 0.58 |
| NSCs | 1 | 56.74 | 0.40 |
| | 2 | 30.60 | 1.01 |
| | 3 | 56.69 | 0.90 |
| EBs | 1 | 59.37 | 0.94 |
| | 2 | 29.87 | 0.62 |
| | 3 | 57.84 | 0.55 |

Note:
Group 1 was a control group (immunologically non-compatible);
Group 2 was a constructed immunologically compatible cell group; and
Group 3 was an immunologically compatible cell group treated with an inducer (Dox).

It can be seen in Table 17 and FIG. 16 that the immunological compatibility effects of the allogeneic hPSCs and hPSC-derived derivatives (hPSCs-MSCs, NSCs, and EBs) that we prepared were significant, and after being treated with the inducer (Dox), they could all show an effect of non-expression of the knocked-in immunologically compatible molecule sequence.

### Example 11

In this example, the effect of immunological compatibility between the target cells prepared in Example 10 and NK cells was tested by the ⁵¹Cr release assay.

1. ⁵¹Cr-labeled target cells and NK cells were added to a 96-well culture plate at a ratio of 1:5 for post-reaction detection; and

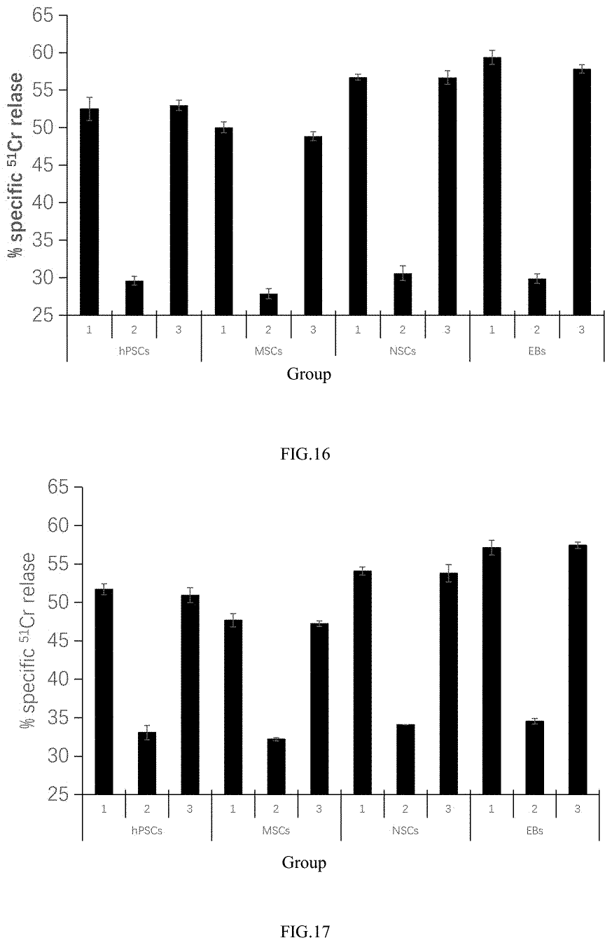

2. the cell-specific ⁵¹Cr release rate of the target cells was detected according to the ⁵¹Cr release assay, and the results are shown in Table 18 and FIG. 17.

TABLE 18
⁵¹Cr release rates of hPSC-MSC, NSC and EB immunologically
compatible cells

| | | Mean ⁵¹Cr | |
|---|---|---|---|
| | Group | release rate (%) | Deviation (±) |
| hPSCs | 1 | 51.71 | 0.70 |
| | 2 | 33.06 | 0.93 |
| | 3 | 50.94 | 0.98 |
| MSCs | 1 | 47.67 | 0.86 |
| | 2 | 32.19 | 0.20 |
| | 3 | 47.24 | 0.36 |
| NSCs | 1 | 54.08 | 0.53 |
| | 2 | 34.08 | 0.02 |
| | 3 | 53.78 | 1.15 |
| EBs | 1 | 57.13 | 0.96 |
| | 2 | 34.56 | 0.37 |
| | 3 | 57.43 | 0.41 |

Note:
Group 1 was a control group (immunologically non-compatible);
Group 2 was a constructed immunologically compatible cell group; and
Group 3 was an immunologically compatible cell group treated with an inducer (Dox).

It can be seen in Table 18 and FIG. 17 that the immunological compatibility effects of the allogeneic hPSCs and hPSC-derived derivatives (hPSCs-MSCs, NSCs, and EBs) that we prepared were significant, and after being treated with the inducer (Dox), they could all show an effect of non-expression of the knocked-in immunologically compatible molecule sequence.

### Example 12

In this example, by the CFSE test assay, the effect of immunological compatibility between the target cells prepared in Example 10 and T cells was tested.

(1) CFSE-labeled target cells (hPSCs, MSCs, NSCs, and EBs) and T cells were added to a 5 ml flow tube at a ratio of 1:5 for reaction and then detection; and

(2) the percentage of CFSE⁺PI⁺ cells (dead target cells) in each kind of cell was detected according to the CFSE test assay, and the results are shown in Table 19 and FIG. 18.

TABLE 19
CFSE test results of hPSC, MSC, NSC and EB
immunologically compatible cells

| | | Mean death rate | |
|---|---|---|---|
| | Group | of target cell (%) | Deviation (±) |
| hPSCs | 1 | 54.73 | 0.16 |
| | 2 | 28.96 | 0.39 |
| | 3 | 53.85 | 0.88 |
| MSCs | 1 | 50.53 | 1.33 |
| | 2 | 28.44 | 0.69 |
| | 3 | 50.12 | 0.61 |
| NSCs | 1 | 56.80 | 0.72 |
| | 2 | 30.38 | 0.23 |
| | 3 | 56.61 | 1.12 |
| EBs | 1 | 60.27 | 1.26 |
| | 2 | 31.38 | 1.10 |
| | 3 | 59.73 | 1.25 |

Note:
Group 1 was a control group (immunologically non-compatible);
Group 2 was a constructed immunologically compatible cell group; and
Group 3 was an immunologically compatible cell group treated with an inducer (Dox).

It can be seen in Table 19 and FIG. 18 that the immunological compatibility effects of the allogeneic hPSCs and hPSC-derived derivatives (hPSCs-MSCs, NSCs, and EBs) that we prepared were significant, and after being treated with the inducer (Dox), they could all show an effect of non-expression of the knocked-in immunologically compatible molecule sequence.

### Example 13

In this example, the expression of CD₁₀₇ₐ in NK cells was analyzed using the flow cytometer (FCM) to detect the immunological compatibility effect of the target cells prepared in Example 10:

1. the target cells and NK cells were added to a 5 ml flow tube at a ratio of 1:1 for reaction and then detection; and

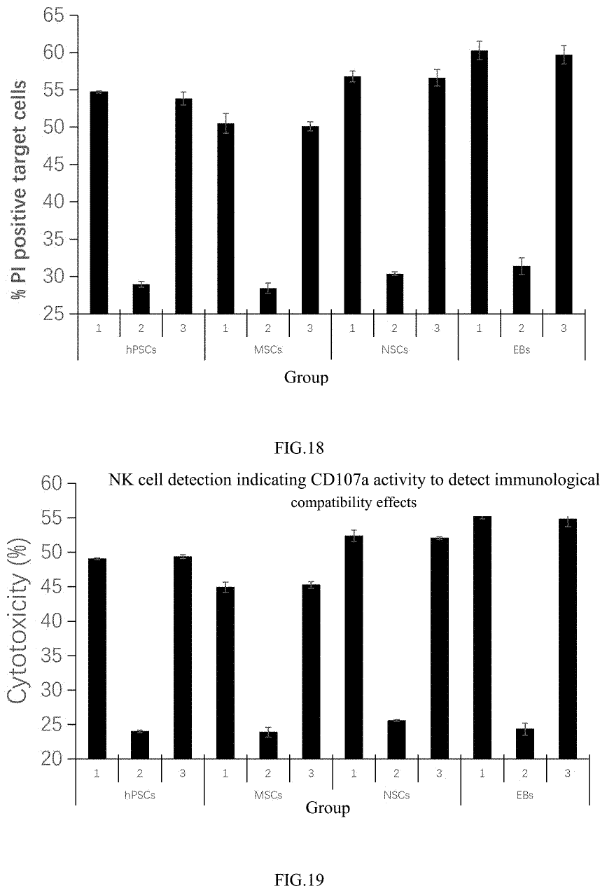

2. the cytotoxicity of the NK cells was detected according to the expression of CD₁₀₇ₐ in the NK cells, and the results are shown in Table 20 and FIG. 19.

TABLE 20
Detection results of CD₁₀₇ₐ expression in NK cells

| | | Mean death rate | |
|---|---|---|---|
| | Group | of NK cell | Deviation (±) |
| hPSCs | 1 | 49.07 | 0.11 |
| | 2 | 24.02 | 0.20 |
| | 3 | 49.36 | 0.27 |
| MSCs | 1 | 44.93 | 0.73 |
| | 2 | 23.90 | 0.72 |
| | 3 | 45.27 | 0.48 |
| NSCs | 1 | 52.37 | 0.86 |
| | 2 | 25.58 | 0.13 |
| | 3 | 52.05 | 0.21 |
| EBs | 1 | 55.60 | 0.77 |
| | 2 | 24.33 | 0.87 |
| | 3 | 54.81 | 1.07 |

Note:
Group 1 was a control group (immunologically non-compatible);
Group 2 was a constructed immunologically compatible cell group; and
Group 3 was an immunologically compatible cell group treated with an inducer (Dox).

It can be seen in Table 20 and FIG. 19 that the immunological compatibility effects of the allogeneic hPSCs and hPSC-derived derivatives (hPSCs-MSCs, NSCs, and EBs) that we prepared were significant, and after being treated with the inducer (Dox), they could all show an effect of non-expression of the knocked-in immunologically compatible molecule sequence.

### Example 14

Through Examples 2 to 9, we selected the scheme of Group D1 for subsequent verification experiments. It was confirmed by Examples 10 to 13 that the immunological compatibility effects of the allogeneic hPSCs and hPSC-derived derivatives (hPSCs-MSCs, NSCs, and EBs) that we prepared were significant, and that when the inducer (Dox) was used, the knocked-in sequence all were not expressed. Since an shRNA/shRNA-miR gene manipulation method was used in some of the experimental schemes, the consequences of overuse of shRNA/shRNA-miR had to be considered.

In this example, the shRNA/miRNA processor complex gene Dhrosha and an anti-interferon effector molecule (PKR-shRNA) were simultaneously inserted into the genomic safe locus of the hPSC, MSC, NSC, and EB immunologically compatible cells prepared in Group D1 of Example 10, wherein Dhrosha was knocked into the position of MCS III in an AAVS1 KI vector, while PKR-shRNA was knocked into the position of the general backbone of shRNA in the AAVS1 KI vector and was seamlessly linked with other shRNAs.

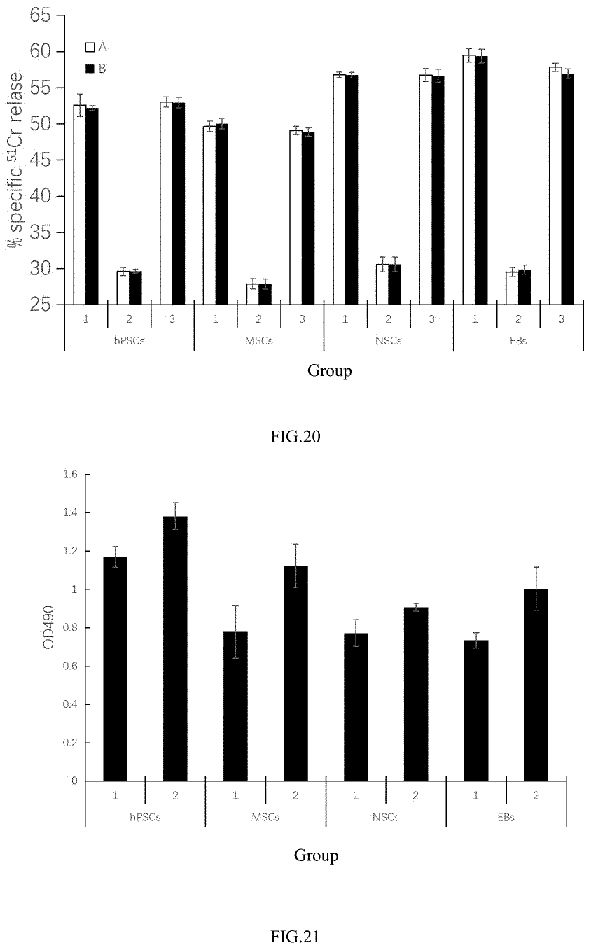

The target cells (hPSCs, MSCs, NSCs, and EBs) and T cells were added to a 96-well culture plate at a ratio of 1:5 for reaction and then detection; and the cell-specific ⁵¹Cr release rates of the hPSC, MSC, NSC and EB immunologically compatible cells were detected according to the ⁵¹Cr release assay, and the results are shown in Table 21 and FIG. 20.

TABLE 21
Cell-specific ⁵¹Cr release rates of hPSC, MSC, NSC and EB
immunologically compatible cells

| | Group | Mean ⁵¹Cr release rate | Deviation (±) |
|---|---|---|---|
| hPSCs | A1 | 52.59 | 1.44 |
| | B1 | 52.22 | 0.32 |
| | A2 | 29.60 | 0.65 |
| | B2 | 29.65 | 0.27 |
| | A3 | 53.02 | 0.78 |
| | B3 | 52.96 | 0.71 |
| MSCs | A1 | 49.65 | 0.33 |
| | B1 | 50.04 | 0.73 |
| | A2 | 27.90 | 0.73 |
| | B2 | 27.86 | 0.69 |
| | A3 | 49.07 | 0.67 |
| | B3 | 48.89 | 0.58 |
| NSCs | A1 | 56.79 | 0.54 |
| | B1 | 56.74 | 0.40 |
| | A2 | 30.58 | 1.03 |
| | B2 | 30.60 | 1.01 |
| | A3 | 56.76 | 1.08 |
| | B3 | 56.69 | 0.90 |
| EBs | A1 | 59.49 | 0.89 |
| | B1 | 59.37 | 0.94 |
| | A2 | 29.52 | 0.57 |
| | B2 | 29.87 | 0.62 |
| | A3 | 57.84 | 0.55 |
| | B3 | 56.95 | 0.66 |

Note:
A represented a group without knock-in of shRNA-miR processor complex genes and anti-interferon effector molecules; and
B represented a group with knock-in of shRNA-miR processor complex genes and anti-interferon effector molecules.
1 was a control group (immunologically non-compatible);
2 was an immunologically compatible cell group which was constructed; and
3 was an immunologically compatible cell group treated with an inducer (Dox).

It can be seen in Table 21 and FIG. 20 that the immunological compatibility effects of the immunologically compatible cells of allogeneic hPSCs and hPSC-derived derivatives (hPSCs-MSCs, NSCs, and EBs) that we prepared were not affected by the knocked-in shRNA-miR processor complex genes and anti-interferon effector molecules.

### Example 15

In this example, the shRNA/miRNA processor complex gene Dhrosha (the expression of which could be inducibly turned off) and an anti-interferon effector molecule (PKR-shRNA) were simultaneously inserted into the genomic safe loci of the hPSC, MSC, NSC, and EB immunologically compatible cells prepared in Group D1 of Example 10, and the effects thereof on the activity of the immunologically compatible cells were detected.

The MTT method was used to detect cell activity. 3000 cells were separately taken into a 96-well plate and cultured for 72 h before detection. The results are shown in Table 22 and FIG. 21.

TABLE 22
Cell activity

| | | | | Independent sample t- |
|---|---|---|---|---|
| | | Mean MTT | Deviation | test (n = 5, *p < 0.01, |
| | Group | value (OD490) | (±) | **p < 0.001) |
| hPSCs | 1 | 1.17 | 0.05 | ** |
| | 2 | 1.38 | 0.07 | |
| MSCs | 1 | 0.78 | 0.14 | * |
| | 2 | 1.12 | 0.11 | |
| NSCs | 1 | 0.77 | 0.07 | * |
| | 2 | 0.91 | 0.02 | |
| EBs | 1 | 0.73 | 0.04 | ** |
| | 2 | 1.00 | 0.11 | |

Note:
1 represented a group without knock-in of shRNA-miR processor complex genes and anti-interferon effector molecules; and
2 represented a group with knock-in of shRNA-miR processor complex genes and anti-interferon effector molecules.

It can be seen in Table 22 and FIG. 21 that after the immunologically compatible cells were knocked-in with shRNA/miRNA processor complex genes and related genes and anti-interferon effector molecules, the activity of the immunologically compatible cells was significantly improved. It was indicated that the immunologically compatible cells, which were inserted with shRNA/shRNA-miR immunologically compatible molecules, would take up excess regulatory processor complex genes and anti-interferon effector molecules. Therefore, when preparing immunologically compatible cells, it was preferable to simultaneously knock in shRNA/miRNA processor complex genes and related genes and anti-interferon effector molecules.

### Example 16

The present disclosure further tested the effects of different shRNA/miRNA processor complex genes and anti-interferon effector molecules on the immunological compatibility of immunologically compatible cells.

The scheme of the immunologically compatible molecules of Group D1 was used for verification test. While knocking in immunologically compatible molecules at the genomic safe locus of EBs (which were representatives of hPSCs derivatives), shRNA/miRNA processor complex genes and/or anti-interferon effector molecules were knocked-in to detect the effects thereof on the activity of the immunologically compatible cells. The experimental groupings are shown in Table 23.

The shRNA/miRNA processor complex gene was knocked into the position of MCS III in the AAV KI vector, while the anti-interferon effector molecule was knocked into the position of the general backbone of shRNA in the AAVS1 KI vector and was seamlessly linked with other shRNAs.

The MTT method was used to detect cell activity. 3000 cells were separately taken into a 96-well plate and cultured for 72 h before detection. The detection results are shown in Table 24.

TABLE 23
Experimental grouping

| Processor | | | | | | | | | | | | | | | | |
|---|---|---|---|---|---|---|---|---|---|---|---|---|---|---|---|---|
| complex | | | | | | | | | | | | | | | | |
| genes | | | | | | | | | | | | | | | | |
| and anti- | | | | | | | | | | | | | | | | |
| interferon | | | | | | | | | | | | | | | | |
| effector | | | | | | | | | | | | | | | | |
| molecules | 1 | 2 | 3 | 4 | 5 | 6 | 7 | 8 | 9 | 10 | 11 | 12 | 13 | 14 | 15 | 16 |
| Dhrosha | | + | | | | | | | | | | | | + | | + |
| Ago1 | | | + | | | | | | | | | | | + | | + |
| Ago2 | | | | + | | | | | | | | | | + | | + |
| Dicer1 | | | | | + | | | | | | | | | + | | + |
| Exportin-5 | | | | | | + | | | | | | | | + | | + |
| TRBP | | | | | | | + | | | | | | | + | | + |
| (TARBP2) | | | | | | | | | | | | | | | | |
| PACT | | | | | | | | + | | | | | | + | | + |
| (PRKRA) | | | | | | | | | | | | | | | | |
| DGCR8 | | | | | | | | | + | | | | | + | | + |
| anti-PKR | | | | | | | | | | + | | | | | + | + |
| shRNA | | | | | | | | | | | | | | | | |
| anti-2-5As | | | | | | | | | | | + | | | | + | + |
| shRNA | | | | | | | | | | | | | | | | |
| anti-IRF-3 | | | | | | | | | | | | + | | | + | + |
| shRNA-miR | | | | | | | | | | | | | | | | |
| anti-IRF-7 | | | | | | | | | | | | | + | | + | + |
| shRNA-miR | | | | | | | | | | | | | | | | |

Note:
“+” represented knocked-in shRNA/miRNA processor complex gene and anti-interferon effector molecule. All of Groups 1-16 were knocked in with immunologically compatible molecules.

TABLE 24
Experiment results

| | Mean MTT | | Independent sample t-test |
|---|---|---|---|
| Group | value (OD490) | Deviation (±) | (*p < 0.01, **<0.001) |
| 1 | 0.53 | 0.04 | — |
| 2 | 0.58 | 0.06 | |
| 3 | 0.69 | 0.07 | ** |
| 4 | 0.57 | 0.07 | |
| 5 | 0.53 | 0.02 | |
| 6 | 0.75 | 0.07 | ** |
| 7 | 0.62 | 0.11 | |
| 8 | 0.64 | 0.05 | * |
| 9 | 0.52 | 0.11 | |
| 10 | 0.68 | 0.11 | * |
| 11 | 0.66 | 0.13 | |
| 12 | 0.50 | 0.07 | |
| 13 | 0.55 | 0.05 | |
| 14 | 0.78 | 0.10 | ** |
| 15 | 0.85 | 0.09 | ** |
| 16 | 1.36 | 0.07 | ** |

The experimental results indicated that the simultaneous knock-in of the shRNA/miRNA processor complex genes and/or anti-interferon effector molecules could enhance the activity of the constructed immunologically compatible cells.

### Example 17

The present disclosure further tested the effects of different inducible expression systems and safe loci on the immunological compatibility of immunologically compatible cells.

The scheme of the immunologically compatible molecules of Group D1 was used for verification experiments. The safe loci and reversible turn-off expression systems were tested on hPSC-derived EBs, and the immunological compatibility thereof with T cells was tested. The experimental groupings are shown in Table 25.

The prepared target cells and T cells were added to a 96-well culture plate at a ratio of 1:5 for reaction and then detection; and the effects of different safe loci and different inducible gene expression systems on the cell-specific ⁵¹Cr release rate of the EB immunologically compatible cells were detected according to the ⁵¹Cr release assay, and the detection results are shown in Table 26.

TABLE 25
Experimental grouping

| | | | Inducible gene | Was inducible |
|---|---|---|---|---|
| Group | | Safe locus | expression system | expression system on? |
| 1 | A | AAVS1 | tet-Off | No |
| | B | AAVS1 | tet-Off | Yes |
| 2 | A | AAVS1 | Dimer | No |
| | B | AAVS1 | Dimer | Yes |
| 3 | A | eGSH | tet-Off | No |
| | B | eGSH | tet-Off | Yes |
| 4 | A | eGSH | Dimer | No |
| | B | eGSH | Dimer | Yes |
| 5 | A | H11 | tet-Off | No |
| | B | H11 | tet-Off | Yes |
| 6 | A | H11 | Dimer | No |
| | B | H11 | Dimer | Yes |

TABLE 26
⁵¹Cr release rate of each experimental group

| | | Mean ⁵¹Cr | | Independent sample t- |
|---|---|---|---|---|
| Group | | release rate | Deviation (±) | test (*p < 0.01) |
| 1 | A | 30.09 | 0.19 | * |
| | B | 58.44 | 1.07 | |
| 2 | A | 30.54 | 0.37 | * |
| | B | 58.63 | 0.54 | |
| 3 | A | 30.22 | 0.05 | * |
| | B | 59.35 | 1.04 | |
| 4 | A | 31.12 | 0.11 | * |
| | B | 59.36 | 1.18 | |
| 5 | A | 31.18 | 1.68 | * |
| | B | 58.99 | 1.07 | |
| 6 | A | 29.10 | 0.94 | * |
| | B | 58.93 | 0.23 | |

The above experimental data indicated that the different safe loci did not affect the expression of the inserted gene, and the different inducible gene expression systems also did not affect the expression of the inserted gene, i.e., not affecting immunological compatibility; in addition, the inducible gene expression systems could play a reversible turn-off function.

### Example 18

In the present disclosure, effects were tested, wherein the concentration of an exogenous inducer was adjusted so that the transplant gradually expresses a low concentration of HLA molecules to stimulate the body. Therefore, the body gradually developed tolerance to the transplant and finally achieved stable tolerance.

The immunologically compatible EBs of Group D1 in Example 2 were used for a verification test, and the test was carried out in EBs (which were representatives of hPSCs derivatives). The immunologically compatible EBs were divided into 2 groups. The immunologically compatible EBs of Group 2 were co-cultured with T cells at a ratio of 1:1, and the added amount of Dox was gradually increased according to the following table. After the inducer induced the EBs to develop tolerance, the ⁵¹Cr release assay and karyotyping assay were performed.

TABLE 27
Added concentration of Dox

| | Concentration | | | |
|---|---|---|---|---|
| No. | of Dox | | T cells added? | Culture time |
| 1 | 0.1875 | uM | Yes | One week |
| 2 | 0.375 | uM | Yes | One week |
| 3 | 0.75 | uM | Yes | One week |
| 4 | 1.5 | uM | Yes | One week |
| 5 | 3 | uM | Yes | One week |
| 6 | 6 | uM | Yes | One week |

By the ⁵¹Cr release assay, the effect of immunological compatibility between immunologically compatible EBs and T cells of the two groups was tested. Flow cytometry sorting was performed first, co-cultured T cells were removed, an inducer (Dox, 6 μM) was then added to both the immunologically compatible EBs of Group 1 (which were not induced with Dox for tolerance), and the immunologically compatible EBs of Group 2 (which were gradually induced with Dox over 6 weeks for tolerance), for 48 h of treatment, then the cells were digested into individual cells as target cells; and after labeling the target cells (EBs) with ⁵¹Cr, the target cells and T cells were added to a 96-well culture plate at a ratio of 1:5 for reaction and then detection, and whether the immunologically compatible EBs developed immune tolerance was detected according to the ⁵¹Cr release assay.

TABLE 28
Immune tolerance effect of EBs

| | | | | Independent |
|---|---|---|---|---|
| | | Mean 51Cr | Deviation | sample t-test |
| Group | | release rate | (±) | (*p < 0.01) |
| 1 | Immunologically compatible | 58.44 | 1.07 | * |
| | EBs without Dox-induction for | | | |
| | tolerance | | | |
| 2 | Immunologically compatible | 30.09 | 0.19 | |
| | EBs gradually induced with | | | |
| | Dox over 6 weeks for | | | |
| | tolerance | | | |

TABLE 29
Karyotyping assay

| | | Chromosome | Karyotype |
|---|---|---|---|
| Group | | mitosis phase | abnormality rate |
| 1 | Immunologically compatible EBs | 100 | 5% |
| | without Dox-induction for | | |
| | tolerance | | |
| 2 | Immunologically compatible EBs | 100 | 0 |
| | gradually induced with Dox over 6 | | |
| | weeks for tolerance | | |

The experimental results indicated that the transplant could develop tolerance by gradually adjusting the amount of the inducer (Dox) used. In addition, after tolerance is developed, it was beneficial for the own immune system to eliminate mutants from the transplant, making the transplant safer.

The above embodiments are only preferences of the present disclosure, and those skilled in the art can understand that without departing from the principle and gist of the present disclosure, modifications and replacements with equivalent effects on these embodiments all fall within the scope of protection of the claims of the present disclosure.

1. A pluripotent stem cell or a derivative thereof, wherein an inducible gene expression system and an expression sequence of at least one immunologically compatible molecule are introduced into the genome of the pluripotent stem cell or the derivative thereof;
the immunologically compatible molecule is used to regulate the expression of immune response-related genes in the pluripotent stem cell or the derivative thereof; and
the expression of the immunologically compatible molecule is regulated by the inducible gene expression system.

2. The pluripotent stem cell or the derivative thereof according to claim 1, wherein the to inducible gene expression system is regulated by an exogenous inducer; and the on and off state of the inducible gene expression system is controlled by adjusting the added amount, duration of action, and type of the exogenous inducer so as to control the expression amount of the expression sequence of the immunologically compatible molecule.

3. The pluripotent stem cell or the derivative thereof according to claim 2, wherein the inducible gene expression system includes at least one selected from the group consisting of the Tet-Off system and dimer-induced expression system.

4. The pluripotent stem cell or the derivative thereof according to claim 1, wherein the immune response-related genes include:
(1) major histocompatibility complex genes, including at least one selected from the group consisting of HLA-A, HLA-B, HLA-C, HLA-DRA, HLA-DRB1, HLA-DRB3, HLA-DRB4, HLA-DRB5, HLA-DQA1, HLA-DQB1, HLA-DPA1 and HLA-DPB1;
(2) major histocompatibility complex-related genes, including at least one selected from the group consisting of B2M and CIITA; and
(3) T cell surface negative costimulatory molecules, including at least one selected from the group consisting of CTLA4, PD-1 and BTLA.

5. The pluripotent stem cell or the derivative thereof according to claim 1, wherein the immunologically compatible molecule includes at least one selected from the group consisting of:
(1) negative costimulatory molecules on the antigen-presenting cell surface;
(2) activating antibodies against “negative costimulatory molecules on the T cell surface” located on the transplanted cell surface, wherein the “negative costimulatory molecules on the T cell surface” include at least one selected from the group consisting of CTLA4, PD-1 and BTLA;
(3) stimulating ligands against “negative costimulatory molecules on the T cell surface” located on the transplanted cell surface;
(4) immune tolerance-related genes;
(5) HLA-C class molecules;
(6) shRNAs and/or shRNA-miRs for major histocompatibility complex genes; and
(7) shRNAs and/or shRNA-miRs for major histocompatibility complex-related genes.

6. The pluripotent stem cell or the derivative thereof according to claim 5, wherein
(1) the negative costimulatory molecules on the antigen-presenting cell surface include at least one selected from the group consisting of PD-L1, Siglec-15, B7-H4 and B7-H5;
(2) the activating antibodies against the “negative costimulatory molecules on the T cell surface” located on the transplanted cell surface are membrane antibodies;
(3) the stimulating ligands against the “negative costimulatory molecules on the T cell surface” located on the transplanted cell surface include at least one selected from the group consisting of PD-L1, Siglec-15, B7-H4 and B7-H5;
(4) the immune tolerance-related genes include at least one selected from the group consisting of CD47 and HLA-G;
(5) the HLA-C class molecules include HLA-C multiple alleles with a proportion of more than 90% in total in the human population, or fusion protein genes composed of the HLA-C multiple alleles with a proportion of more than 90% in total in the human population and B2M;
(6) the major histocompatibility complex genes include at least one selected from the group consisting of HLA-A, HLA-B, HLA-C, HLA-DRA, HLA-DRB1, HLA-DRB3, HLA-DRB4, HLA-DRB5, HLA-DQA1, HLA-DQB1, HLA-DPA1 and HLA-DPB1; and
(7) the major histocompatibility complex-related genes include at least one selected from the group consisting of B2M and CIITA.

7. The pluripotent stem cell or the derivative thereof according to claim 5, wherein
target sequences of the shRNAs and/or shRNA-miRs for the major histocompatibility complex genes are at least one selected from the group consisting of SEQ ID NO. 14 to SEQ ID NO. 23; and
target sequences of the shRNAs and/or shRNA-miRs for the major histocompatibility complex-related genes are at least one selected from the group consisting of SEQ ID NO. 1 to SEQ ID NO. 13.

8. The pluripotent stem cell or the derivative thereof according to claim 1, wherein an shRNA and/or miRNA processor complex-related gene and/or an anti-interferon effector molecule are further introduced into the genome of the pluripotent stem cell or the derivative thereof.

9. The pluripotent stem cell or the derivative thereof according to claim 8, wherein the shRNA and/or miRNA processor complex-related gene includes at least one selected from the group consisting of Dhrosha, Ago1, Ago2, Dicer1, Exportin-5, TRBP (TARBP2), PACT (PRKRA) and DGCR8; and the anti-interferon effector molecule is shRNA and/or shRNA-miR for at least one selected from the group consisting of PKR, 2-5As, IRF-3 and IRF-7.

10. The pluripotent stem cell or the derivative thereof according to claim 9, wherein target sequences of the shRNAs and/or shRNA-miRs for PKR, 2-5As, IRF-3 or IRF-7 are at least one selected from the group consisting of SEQ ID NO. 31 to SEQ ID NO. 90.

11. The pluripotent stem cell or the derivative thereof according to claim 7 or 10, wherein a general backbone sequence of the shRNAs or shRNA-miRs for major histocompatibility complex genes, major histocompatibility complex-related genes, and PKR, 2-5As, IRF-3 or IRF-7 is as shown below:
(1) general backbone sequence of shRNA: comprising, in sequence from 5′ to 3′, an shRNA target sequence, a stem-loop sequence, and a reverse complement sequence of the shRNA target sequence; wherein the shRNA target sequence is as defined in claim 7 or 10, and the length of the loop sequence is 3-9 bases; and
(2) general backbone sequence of shRNA-miR: obtained by replacing a target sequence in microRNA-30 or microRNA-155 with the shRNA-miR target sequence as defined in claim 7 or 10.

12. The pluripotent stem cell or the derivative thereof according to any one of claims 1-11, wherein the inducible gene expression system, the expression sequence of the immunologically compatible molecule, the shRNA and/or miRNA processor complex-related gene, and the anti-interferon effector molecule are introduced by means of viral vector interference, non-viral vector transfection or gene editing.

13. The pluripotent stem cell or the derivative thereof according to claim 12, wherein the gene editing is gene knock-in.

14. The pluripotent stem cell or the derivative thereof according to any one of claims 1-11, wherein introduction loci for the inducible gene expression system, the expression sequence of the immunologically compatible molecule, the shRNA and/or miRNA processor complex-related gene, and the anti-interferon effector molecule are genomic safe loci.

15. The pluripotent stem cell or the derivative thereof according to claim 14, wherein the genomic safe loci include at least one selected from the group consisting of the AAVS1 safe locus, the eGSH safe locus, and the H11 safe locus.

16. The pluripotent stem cell or the derivative thereof according to any one of claims 1-15, wherein the pluripotent stem cell includes an embryonic stem cell, an embryonic germ cell, an embryonic carcinoma cell, or an induced pluripotent stem cell;
the derivative includes a pluripotent stem cell-derived three-germ-layer-derived organ, tissue or cell; and
the pluripotent stem cell-derived three-germ-layer-derived cell includes a mesenchymal stem cell, a neural stem or progenitor cell, or other adult stem cell.

17. A method for preparing an immunologically compatible and reversible universal pluripotent stem cell or a derivative thereof, comprising by introducing an inducible gene expression system and an expression sequence of at least one immunologically compatible molecule into the genome of the pluripotent stem cell or the derivative thereof; the immunologically compatible molecule is used to regulate the expression of immune response-related genes in the pluripotent stem cell or the derivative thereof.

18. The method for preparing an immunologically compatible and reversible universal pluripotent stem cell or a derivative thereof according to claim 17, wherein the immunologically compatible molecule is as defined in any one of claims 5-7.

19. The method for preparing an immunologically compatible and reversible universal pluripotent stem cell or a derivative thereof according to claim 17, wherein the inducible gene expression system includes at least one selected from the group consisting of the Tet-Off system and dimer-induced expression system.

20. The method for preparing an immunologically compatible and reversible universal pluripotent stem cell or a derivative thereof according to claim 17, wherein an shRNA and/or miRNA processor complex-related gene and/or an anti-interferon effector molecule are further introduced into the genome of the pluripotent stem cell or the derivative thereof, wherein the shRNA and/or miRNA processor complex-related gene is as defined in claim 9; and the anti-interferon effector molecule is as defined in any one of claims 9-11.

21. The method for preparing an immunologically compatible and reversible universal pluripotent stem cell or a derivative thereof according to claim 17, wherein introduction loci for the inducible gene expression system, immunologically compatible molecule, shRNA and/or miRNA processor complex-related gene, and anti-interferon effector molecule are genomic safe loci; and the genomic safe loci include at least one selected from the group consisting of the AAVS1 safe locus, the eGSH safe locus, and the H11 safe locus.

22. Use of the pluripotent stem cell or the derivative thereof according to any one of claims 1-16 in the preparation of a product for cellular therapy.

23. Use of the pluripotent stem cell or the derivative thereof according to any one of claims 1-16 in the preparation of a product for organ transplantation.

24. Use of the pluripotent stem cell or the derivative thereof according to any one of claims 1-16 in the construction of a universal PSC cell bank.

25. Use of the pluripotent stem cell or the derivative thereof according to any one of claims 1-16 as a gene drug carrier.
